(19) United States
(12) Patent Application Publication
(10) Pub. No.: US 2009/0221423 A1
(43) Pub. Date: Sep. 3, 2009

(54) Methods to Use 3-Pyridyl Derivatives as Pesticides

(75) Inventors: Michael Puhl, Lampertheim (DE); Matthias Pohlman, Freinsheim (DE); Liliana Parra Rapado, Offenburg (DE); Michael Rack, Eppelheim (DE); Thomas Schmidt, Neustadt (DE); Delphine Breuninger, Bobenheim-Roxheim (DE); Deborah L. Culbertson, Fuquay Varina, NC (US); Douglas D. Anspaugh, Apex, NC (US); Henry Van Tuyl Cotter, Raleigh, NC (US); Hassan Oloumi-Sadeghi, Raleigh, NC (US); David G. Kuhn, Apex, NC (US)

Correspondence Address: BRINKS, HOFER, GILSON & LIONE, P.O. BOX 1340, MORRISVILLE, NC, 27560, US

(73) Assignee: BASF SE, Ludwigshafen (DE)

(21) Appl. No.: 12/162,044

(22) PCT Filed: Jan. 19, 2007
(86) PCT No.: PCT/EP07/50522

§ 371 Date: Nov. 11, 2008

Related U.S. Application Data
(60) Provisional application No. 60/762,305, filed on Jan. 26, 2006.
(60) Provisional application No. 60/867,287, filed on Nov. 27, 2006.
(60) Provisional application No. 60/867,637, filed on Nov. 29, 2006.

(51) Int. Cl. A01N 43/40; A01N 25/26; C07D 401/14; C07D 401/02; A01N 57/10; A01N 43/66

(52) U.S. Cl. 504/100; 514/333; 514/343; 546/256; 546/279.1; 546/268.1; 514/340; 514/143; 514/245; 514/341

(57) ABSTRACT

Pesticidal compositions comprising

3-pyridyl derivatives of formula I

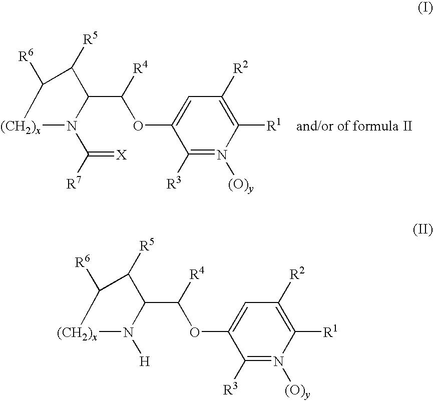

wherein the variables and indices are as defined in the description, and an agronomically acceptable carrier, processes for the preparation of compounds I and use of compounds I or II and the compositions comprising them for combating pests.

The present invention relates to pesticidal compositions comprising

(a) 3-pyridyl derivatives of formula I

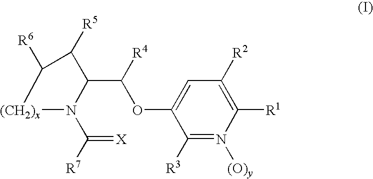

wherein
X is oxygen or sulfur;
R¹ and R² are each independently hydrogen, halogen, cyano, nitro, or
   C₁-C₆-alkyl, C₁-C₆-haloalkyl, C₂-C₅-alkenyl, C₂-C₆-haloalkenyl, C₂-C₆-alkynyl, C₂-C₆-haloalkynyl, C₃-C₆-cycloalkyl, C₃-C₆-halocycloalkyl, C₃-C₆-cycloalkenyl, C₃-C₆-halocycloalkenyl, C₁-C₆-alkyl-C₃-C₆-cycloalkyl, wherein the carbon atoms in these groups may be substituted with 1 to 3 groups selected from Rⁱ, ORⁱ, SRⁱ, S(═O)Rⁱ, S(═O)₂Rⁱ, NRⁱRⁱ, C(═O)ORⁱ, SiRⁱ_{z}R^{j}_{3-z} is 0 to 3), or
   ORⁱ, SRⁱ, S(═O)Rⁱ, S(═O)₂Rⁱ, NRⁱR^{j}, C(═O)ORⁱ, SiRⁱ_{z}R^{j}_{3-z} (z is 0 to 3), or
   phenyl or a 5- to 6-membered heteroaromatic ring which may contain 1 to 4 heteroatoms selected from oxygen, nitrogen and sulfur, or a 3- to 7-membered saturated or partially unsaturated heterocyclic ring which may contain 1 to 3 heteroatoms selected from oxygen, nitrogen and sulfur,
   wherein the carbon atoms in phenyl or in the heteroaromatic or heterocyclic ring may be substituted with 1 to 3 groups selected from halogen, amino, cyano, Rⁱ, ORⁱ, SRⁱ and nitro;
   Rⁱ, R^{j} are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₈-halocycloalkyl, C₃-C₆-cycloalkenyl, C₃-C₆-halocycloalkenyl, C₁-C₆-haloalkoxy, C₁-C₄-alkoxy-C₁-C₆-alkyl, C₁-C₄-alkylthio-C₁-C₆-alkyl, C₁-C₄-alkylsulfinyl-C₁-C₆-alkyl, C₁-C₄-alkylsulfonyl-C₁-C₆-alkyl, C₁-C₄-haloalkoxy-C₁-C₆-alkyl, C₁-C₄-haloalkylthio-C₁-C₆-alkyl, (C₁-C₄-alkoxy)carbonyl-C₁-C₆-alkyl, (C₁-C₄-alkyl)amino-C₁-C₆-alkyl, di(C₁-C₄-alkyl)amino-C₁-C₆-alkyl, C₃-C₆-cycloalkyl-C₁-C₆-alkyl, phenyl-C₁-C₆-alkyl, or C₁-C₆-alkyl which is substituted with 1 to 3 cyano groups;
R³ is hydrogen, halogen or C₁-C₆-alkyl;
R⁴ is hydrogen or C₁-C₆-alkyl;
R⁵ and R⁶ are each independently hydrogen, halogen, cyano, or C₁-C₆-alkyl;
R⁷ is C₁-C₆-alkyl, C₂-C₆-alkenyl, C₂-C₆-alkynyl, C₃-C₆-cycloalkyl, C₃-C₆-cycloalkenyl, C₁-C₆-haloalkyl, C₂-C₆-haloalkenyl, C₂-C₆-haloalkynyl, C₃-C₆-halocycloalkyl, C₃-C₆-halocycloalkenyl, or —C(=G)R^{a}, —C(=G)OR^{a}, —C(=G)NR^{a}R^{b}, —C(═NOR^{a})R^{b}, C₃-C₆-cycloalkyl-C₁-C₆-alkyl;
   G is oxygen or sulfur;
   R^{a}, R^{b} are each independently halogen, cyano, nitro, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₆-halocycloalkyl, C₃-C₆-cycloalkenyl, C₃-C₆-halocycloalkenyl, OR^{k}, SR^{k}, S(═O)R^{k}, S(═O)₂R^{k}, S(═O)₂NR^{k}R^{m}, C(═O)R^{k}, C(═O)OR^{k}, C(═O)NR^{k}R^{m}, C(═NOR^{k})R^{m}, —C(=G)NR^{k}—NR^{m}Rⁿ, SiR^{k}_{z}R^{m}_{3-z} (z is 0 to 3), or
   R^{a}, R^{b} are each independently phenyl or a 5- to 6-membered heteraromatic ring which may contain 1 to 4 heteroatoms selected from oxygen, nitrogen and sulfur, or a 4- to 7-membered saturated or partially unsaturated heterocyclic ring which may contain 1 to 3 heteroatoms selected from oxygen, nitrogen and sulfur, wherein the carbon atoms in phenyl or in the heteroaromatic or heterocyclic ring may be substituted with 1 to 5 halogens;
      R^{k}, R^{m}, Rⁿ are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₈-halocycloalkyl, C₃-C₆-cycloalkenyl, C₃-C₆-halocycloalkenyl; or
R⁷ is phenyl or a 3- to 7-membered saturated or partially unsaturated heterocyclic ring which may contain 1 to 3 heteroatoms selected from oxygen, sulfur and nitrogen or a 5- to 6-membered heteroaromatic ring system which may contain 1 to 4 heteroatoms selected from oxygen, nitrogen and sulfur, which phenyl, heterocyclic ring or heteroaromatic ring is bonded via a carbon atom of the ring and which phenyl, heterocyclic ring or heteroaromatic ring may be bonded via C₁-C₄-alkyl,
   wherein phenyl or the heteroaromatic ring or the heterocyclic ring may be fused to a ring selected from phenyl and a 5- to 6-membered saturated, partially unsaturated or aromatic heterocyclic ring which may contain 1 to 3 heteroatoms selected from oxygen, nitrogen and sulfur,
   wherein the above groups R⁷ are unsubstituted, or the hydrogen atoms in these groups may all or in part be replaced with any combination of groups selected from R^{c};
   R^{c} is each independently halogen, cyano, nitro, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₈-halocycloalkyl, C₃-C₆-cycloalkenyl, C₃-C₈-halocycloalkenyl, OR^{k}, SR^{k}, S(═O)R^{k}, S(═O)₂R^{k}, NR^{k}R^{m}, N(OR^{k})R^{m}, —S(═O)₂NR^{k}R^{m}, C(═O)R^{k}, C(═O)OR^{k}, C(═O)NR^{k}R^{m}, C(═NOR^{k})R^{m}, —NR^{k}C(=G)R^{m}, —N(OR^{k})C(=G)R^{m}, —N[C(=G)R^{k}][C(=G)R^{m}], —NR^{k}C(=G)OR^{m}, —N(OR^{k})C(=G)OR^{m}, —C(=G)NR^{k}—NR^{m}Rⁿ, —NR^{k}SO₂R^{m}, SiR^{k}_{z}R^{m}_{3-z} (z is 0 to 3), or
   R^{c} is each independently phenyl or a 5- to 6-membered heteraromatic ring which may contain 1 to 4 heteroatoms selected from oxygen, nitrogen and sulfur, or a 4- to 7-membered saturated or partially unsaturated heterocyclic ring which may contain 1 to 3 heteroatoms selected from oxygen, nitrogen and sulfur, wherein the carbon atoms in phenyl or in the heteroaromatic or heterocyclic ring may be substituted with 1 to 5 halogens;
      R^{k}, R^{m}, Rⁿ are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₈-halocycloalkyl, C₃-C₆-cycloalkenyl, C₃-C₆-halocycloalkenyl;
x is 0 or 1;
y is 0 or 1;
   or the diastereomers, enantiomers or salts thereof;
   and/or comprising
   (a) 3-pyridyl derivatives of formula II

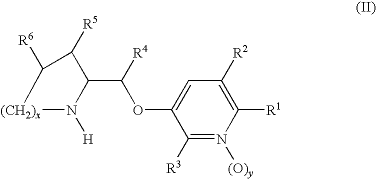

wherein the variables and indices have the meaning as defined above for compounds (I), with the exception of compounds wherein
x, y are zero;
R⁴ is hydrogen or C₁-C₄-alkyl;
R¹, R² are each independently hydrogen, halogen, cyano, C₁-C₄-alkyl, C₁-C₄-haloalkyl, C₁-C₄-alkoxy, C₁-C₄-alkylthio, C₁-C₄-alkylsulfinyl, C₁-C₄-alkylsulfonyl, C₁-C₄-haloalkylthio, C₁-C₄-haloalkylsulfinyl, C₁-C₄-haloalkylsulfonyl; and
R³, R⁵, R⁶ are hydrogen;
   or the diastereomers, enantiomers or salts thereof, and
   (b) an agronomically acceptable carrier.

Depending on the substitution pattern, the compounds of formula I or II can contain one or more chiral centers, in which case they are present as enantiomer or diastereomer mixtures. Subject-matter of this invention are not only compositions containing these mixtures but also those containing the pure enantiomers or diastereomers.

In addition, the present invention relates to processes for preparing the compounds I, methods for the control of pests by contacting the pest or their food supply, habitat, breeding ground or locus with a pesticidally effective amount of compounds or compositions of formula I or II, and to some new compounds of formula I.

Moreover, the present invention also relates to a method of protecting growing plants from attack or infestation by insects or acarids by applying to the plants, or to the soil or water in which they are growing, a pesticidally effective amount of compositions or compounds of formula I or II.

In spite of the commercial pesticides available today, damage to crops, both growing and harvested, caused by insects and nematodes still occurs. Therefore, there is continuing need to develop new and more effective pesticidal agents.

It was therefore an object of the present invention to provide new pesticidal compositions, new compounds and new methods for the control of pests and of protecting growing plants from attack or infestation by pests.

We have found that these objects are achieved by the compositions and the compounds of formula I or II.

3-pyridyl derivates of formula I have been generically described in WO 02/89800. WO 98/25920 discloses some 3-pyridyl derivatives which carry an azetidin-2-yl-methoxy group in which the nitrogen atom of the azetidine ring is substituted by hydrogen or a prodrug moiety which may be selected from numerous examples, inter alia forming an amide function together with the nitrogen atom. WO 99/32480 among a large variety of compounds describes 3-pyridyl derivates which carry an azetidin-2-yl-methoxy or pyrrolidin-2-yl-methoxy group in which the nitrogen atom of the azetidine or pyrrolidin ring is substituted by hydrogen, allyl or C₁-C₆-alkyl. It also discloses in a general manner prodrug moieties which may be bonded to the nitrogen atom and which may be selected from numerous examples, inter alia forming an amide function together with the nitrogen atom. WO 99/32480 and WO 98/25920 disclose very few specific compounds which carry a prodrug moiety at the nitrogen atom of the azetidine ring which fall under the definition of compounds I of the present invention.

The pharmaceutical activity of 3-pyridyl derivates of formula II is known from the literature (U.S. Pat. No. 5,629,325, U.S. Pat. No. 6,127,386, U.S. Pat. No. 6,437,138, WO 94 08992, WO 96/40682, WO 97/46554, WO 98/25920, WO 99/32480, WO 05/000806).

Co-pending application PCT/EP2006/004992 describes the pesticidal activity of certain 3-pyridyl derivates which carry an azetidin-2-yl-methoxy group in which the nitrogen atom of the azetidine ring is substituted by hydrogen. The present application does not pertain to the pesticidal activity of these compounds.

A pesticidal activity of compounds of formula I or II has not been known yet.

In this specification and in the claims, reference will be made to a number of terms that shall be defined to have the following meanings:

“Salt” as used herein includes adducts of compounds I with maleic acid, dimaleic acid, fumaric acid, difumaric acid, methane sulfenic acid, methane sulfonic acid, and succinic acid. Moreover, included as “salts” are those that can form with, for example, amines, metals, alkaline earth metal bases or quaternary ammonium bases, including zwitterions. Suitable metal and alkaline earth metal hydroxides as salt formers include the salts of barium, aluminum, nickel, copper, manganese, cobalt zinc, iron, silver, lithium, sodium, potassium, magnesium or calcium. Additional salt formers include chloride, sulfate, acetate, carbonate, hydride, and hydroxide. Desirable salts include adducts of compounds I or II with maleic acid, dimaleic acid, fumaric acid, difumaric acid, and methane sulfonic acid.

“Halogen” will be taken to mean fluoro, chloro, bromo and iodo.

The term “alkyl” as used herein refers to a branched or unbranched saturated hydrocarbon group having 1 to 6 carbon atoms, for example methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl.

The term “haloalkyl” as used herein refers to a straight-chain or branched alkyl group having 1 to 6 carbon atoms (as mentioned above), where some or all of the hydrogen atoms in these groups may be replaced by halogen atoms as mentioned above, for example C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl and pentafluoroethyl;

Similarly, “alkoxy” and “alkylthio” refer to straight-chain or branched alkyl groups having 1 to 6 carbon atoms (as mentioned above) bonded through oxygen or sulfur linkages, respectively, at any bond in the alkyl group. Examples include methoxy, ethoxy, propoxy, isopropoxy, methylthio, ethylthio, propylthio, isopropylthio, and n-butylthio.

Similarly, “alkylsulfinyl” and “alkylsulfonyl” refer to straight-chain or branched alkyl groups having 1 to 6 carbon atoms (as mentioned above) bonded through —S(═O)— or —S(═O)₂-linkages, respectively, at any bond in the alkyl group. Examples include methylsulfinyl and methylsulfonyl.

Similarly, “alkylamino” refers to a nitrogen atom which carries 1 or 2 straight-chain or branched alkyl groups having 1 to 6 carbon atoms (as mentioned above) which may be the same or different. Examples include methylamino, dimethylamino, ethylamino, diethylamino, methylethylamino, isopropylamino, or methylisopropylamino.

The term “alkenyl” as used herein intends a branched or unbranched unsaturated hydrocarbon group having 2 to 10 carbon atoms and a double bond in any position, for example C₂-C₆-alkenyl such as ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl; 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

The term “alkynyl” as used herein refers to a branched or unbranched unsaturated hydrocarbon group having 2 to 10 carbon atoms and containing at least one triple bond, such as ethynyl, propynyl, 1-butynyl, 2-butynyl, and the like.

A 5- or 6-membered heteroaromatic ring which contains 1 to 3 heteroatoms selected from oxygen, nitrogen and sulfur may be a 5-membered heteroaromatic ring containing 1 nitrogen atom and 0 to 2 further heteroatoms independently selected from oxygen, nitrogen and sulfur, such as pyrrol, pyrazol, imidazol, triazol, oxazol, isoxazol, oxadiazol, thiazol, isothiazol, thiadiazol; or a 5-membered heteroaromatic ring containing 1 heteroatom selected from oxygen and sulfur, such as furane or thiophen; or a 6-membered heteroaromatic ring containing 1 or 2 or 3 nitrogen atoms, such as pyridine, pyrazine, pyrimidine, pyridazine or triazine.

A 5- or 6-membered heteroaromatic ring which contains 1 to 3 heteroatoms selected from oxygen, nitrogen and sulfur preferably is oxazol, isoxazol, thiazol, isothiazol, pyridine, thiophen or furan.

When fused to a 5- to 6-membered saturated, partially unsaturated or aromatic heterocyclic ring which may contain 1 to 3 heteroatoms selected from oxygen, nitrogen and sulfur, this fused ring system is e.g. pyrimidotriazolyl or indoyl.

A 4- to 7-membered saturated or partially unsaturated heterocyclic ring which may contain 1 to 3 heteroatoms selected from oxygen, sulfur and nitrogen is e.g. a 4- to 5-membered saturated heterocyclic ring containing 1 nitrogen atom and 0 or 1 further heteroatoms independently selected from sulfur, oxygen and nitrogen, such as morpholine, piperazin, piperidine or pyrrolidine, or a 5-membered saturated heterocyclic ring containing 1 heteroatom selected from oxygen, nitrogen or sulfur, such as tetrahydrofuran, tetrahydrothiophen, tetrahydropyranyl or tetrathiopyranyl.

A 4- to 7-membered saturated or partially unsaturated heterocyclic ring which may contain 1 to 3 heteroatoms selected from oxygen, sulfur and nitrogen preferably is tetrahydrofuran, tetrahydropyranyl, tetrathiopyranyl, or piperidinyl.

When fused to a 5- to 6-membered saturated, partially unsaturated or aromatic heterocyclic ring which may contain 1 to 3 heteroatoms selected from oxygen, nitrogen and sulfur, this fused ring system is e.g. indoline.

Phenyl which is fused to phenyl or a 5- to 6-membered saturated, partially unsaturated or aromatic heterocyclic ring which may contain 1 to 3 heteroatoms selected from oxygen, nitrogen and sulfur is e.g. naphthalin, benzoxazolyl, benzthiazolyl, benzimidazolyl, benzoxadiazolyl, or benzthiadiazolyl.

A 5- to 6-membered saturated, partially unsaturated or aromatic heterocyclic ring which may contain 1 to 3 heteroatoms selected from oxygen, nitrogen and sulfur is e.g. pyridine, pyrimidine, (1,2,4)-oxadiazole, (1,3,4)-oxadiazole, pyrrole, furan, thiophene, oxazole, thiazole, imidazole, pyrazole, isoxazole, 1,2,4-triazole, tetrazole, pyrazine, pyridazine, oxazoline, thiazoline, tetrahydrofuran, tetrahydropyran, morpholine, piperidine, piperazine, pyrroline, pyrrolidine, oxazolidine, thiazolidine. Most preferably, this ring system is dioxolan, furan, oxazol, thiazol, or tetrahydrofuran.

Cycloalkyl: monocyclic 3- to 6-, 8- or 10-membered saturated carbon atom rings, e.g. C₃-C₈-cycloalkyl such as cyclopropyl, cyclobutyl, cyclopentyl, cyclohexyl, cycloheptyl, or cyclooctyl.

Pesticidal compositions for the intended use of the present invention may contain mixtures of compounds I and compounds II, especially of a certain N-acyl compound I and the corresponding amine compound II.

Pesticidal compositions comprising as component (a) compounds I as defined above or compounds II wherein the variables and indices have the meaning as defined above for compounds (I), with the exception of compounds wherein x and y are zero, and R⁴ is hydrogen or C₁-C₆-alkyl, and R¹, R² are each independently hydrogen, halogen, cyano, C₁-C₆-alkyl, C₁-C₆-haloalkyl, C₁-C₆-alkoxy, C₁-C₆-alkylthio, C₁-C₆-alkylsulfinyl, C₁-C₆-alkylsulfonyl, C₁-C₆-haloalkylthio, C₁-C₆-haloalkylsulfinyl, C₁-C₆-haloalkylsulfonyl; and R³, R⁵, R⁶ are hydrogen; or the diastereomers, enantiomers or salts thereof are preferred.

Pesticidal compositions comprising as component (a) compounds I as defined above are especially preferred.

With respect to the intended use of the compounds of formula I and the compositions comprising them, particular preference is given to the following compounds:

Compounds of formula I wherein X, R¹, R², R³, R⁴, R⁵, R⁶, R⁷, x and y are as defined above for compounds of formula I, except compounds wherein the combination of variables is: R¹ is fluoro, R², R³, R⁴, R⁵ and R⁶ are hydrogen, x and y are zero, X is oxygen, and R⁷ is selected from 2-oxo-tetrahydrofuran-4-yl, phenyl which may be substituted in the 4-position with nitro, fluoro, chloro, methyl, carboxymethyl, methoxy, or diethylaminomethyl, and phenyl which is substituted in the 2-position with hydroxymethyl.

Compounds of formula I wherein X, R¹, R², R³, R⁴, R⁵, R⁶, R⁷, x and y are as defined at the outset, of the description, with the proviso that,

if X is oxygen, R¹ is fluoro, y is zero, and R⁷ is selected from 2-oxo-tetrahydrofuran-4-yl, pyrrolidin-1-yl, a phenyl ligand which may be substituted in the 4-position with nitro, fluoro, chloro, methyl, carboxymethyl, methoxy, or diethylaminomethyl, and a phenyl ligand which is substituted in the 2-position with hydroxymethyl,
then either the phenyl ligand must carry at least one further substituent; or
R², R³, R⁴, R⁵ and R⁶ are not all hydrogen; or
x is not zero.

Compounds of formula I wherein X, R¹, R², R³, R⁴, R⁵, R⁶, R⁷, x and y are as defined in any of the two preceding paragraphs or as defined at the outset of the description for compounds of formula I with the proviso that,

if R⁴, R⁵ and R⁶ are hydrogen, x and y are zero, X is oxygen, and R¹ is selected from methyl, cyano, chloro, bromo, fluoro, difluoromethyl or methoxy, and R² is selected from ethenyl, chloro or bromo; or
if R⁴, R⁵ and R⁶ are hydrogen, x and y are zero, X is oxygen and R¹ is hydrogen and R² is selected from methyl, n-propyl, chloro, fluoro, nitro, ethoxy; or
if R⁴, R⁵ and R⁶ are hydrogen, x and y are zero, X is oxygen and R³ is fluoro and R² is hydrogen; or
if R⁴, R⁵ and R⁶ are hydrogen, x and y are zero, X is oxygen and R¹ is fluoro or methyl, then R⁷ is not methyl, 2-hydroxymethylphenyl, 4-diethylaminophenyl, phthalyl methyl ester, or 2-oxo-tetrahydrofuran-4-yl.

Compounds of formula I wherein X, R¹, R², R³, R⁴, R⁵, R⁶, R⁷, x and y are as defined in any of the three preceding paragraphs or as defined at the outset of the description for compounds of formula I, with the proviso that,

if R³ and R² are hydrogen and R¹ is selected from methyl, cyano, chloro, bromo, fluoro, difluoromethyl, and methoxy; or
if R³ and R¹ are hydrogen and R² is selected from methyl, n-propyl, ethenyl, 3-propenyl, chloro, fluoro, nitro, and ethoxy; or
if R³ is hydrogen, R¹ is chloro and R² is selected from methyl, ethenyl, ethynyl, chloro and bromo; or
if R³ is hydrogen, R¹ is fluoro and R² is selected from ethyl, ethenyl, chloro and bromo; or
if R¹ and R² are hydrogen and R³ is chloro or fluoro; or
if R¹ is methyl, R² is bromo and R³ is hydrogen; or
if R¹ is methyl, R² is hydrogen and R³ is fluoro;
then R⁷ is not methyl, 2-hydroxymethylphenyl, 4-diethylaminophenyl, phthalyl methyl ester, or 2-oxo-tetrahydrofuran-4-yl.

An any of the four preceding paragraphs or as defined at the outset of the description with the proviso that if R⁴, R⁵ and R¹ are hydrogen, x and y are zero, X is oxygen, then R⁷ is not trifluoroacetyl.

A compound of formula I wherein X is oxygen, y is zero, x is 0 or 1, R¹ and R² are each independently halogen, cyano, amino, C₁-C₄-alkyl, C₁-C₄-alkoxy, C₁-C₄-haloalkoxy, C₁-C₄-alkylthio, C₁-C₄-alkylsulfonyl, R³, R⁵ and R¹ are hydrogen, R⁴ is hydrogen or methyl, preferably hydrogen, and R⁷ is selected from C₁-C₆-alkyl, C₃-C₆-cycloalkyl, phenyl or a 4- to 7-membered saturated or partially unsaturated heterocyclic ring which may contain 1 to 3 heteroatoms selected from oxygen, sulfur and nitrogen and a 5- to 6-membered heteroaromatic ring system which may contain 1 to 4 heteroatoms selected from oxygen, nitrogen and sulfur.

A compound of formula I wherein R⁷ is phenyl which is disubstituted in the 3,4 or 3,5 or 4,5 position or monosubstituted in the 3 position.

With respect to the intended use of the compounds of formula I and the compositions comprising them, particular preference is given to the following meanings of the substituents, in each case on their own or in combination:

A compound of formula I wherein X is oxygen.

A compound of formula I wherein R¹ and R² are each independently hydrogen, halogen, cyano, nitro, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₈-halocycloalkyl, C₃-C₆-cycloalkenyl, C₃-C₆-halocycloalkenyl, C₁-C₆-alkoxy, C₁-C₆-haloalkoxy, C₁-C₆-alkylthio, C₁-C₆-alkylsulfinyl, C₁-C₆-haloalkylsulfinyl, C₁-C₆-alkylsulfonyl, C₁-C₆-haloalkylsulfonyl, di(C₁-C₆-alkyl)amino, C₁-C₆-alkylamino, C₁-C₆-alkoxycarbonyl, or phenyl or a 5- to 6-membered heteroaromatic ring which may contain 1 to 4 heteroatoms selected from oxygen, nitrogen and sulfur, wherein the carbon atoms in these groups may be substituted with 1 to 3 groups selected from halogen, amino, cyano, hydroxy, mercapto, C₁-C₆-alkyl, C₁-C₆-alkoxy, C₁-C₆-haloalkyl, C₁-C₆-haloalkoxy, C₁-C₆-alkylthio, C₁-C₆-haloalkylthio and nitro.

A compound of formula I wherein R¹ and R² are each independently hydrogen, fluoro, chloro, bromo, cyano, amino, dimethylamino, methoxy, acetylene, vinyl or phenyl, preferably chloro.

A compound of formula I wherein R³ is hydrogen, fluoro or methyl, preferably hydrogen.

A compound of formula I wherein R⁴ is hydrogen or methyl, preferably hydrogen.

A compound of formula I wherein R⁵ and R⁶ are each independently hydrogen, fluoro, chloro, cyano, or methyl, preferably hydrogen.

A compound of formula I wherein either R³ and R⁴ or R³ and R⁵ or R³ and R⁶ or R⁴ and R⁵ or R⁴ and R⁶ or R⁵ and R⁶ are both hydrogen.

A compound of formula I wherein only one variable out of the group R³, R⁴, R⁵ and R⁶ is not hydrogen.

A compound of formula I wherein R³, R⁴, R⁵ and R⁶ are all hydrogen.

A compound of formula I wherein R⁷ is branched C₁-C₆-alkyl, C₃-C₆-cycloalkyl, or C₃-C₆-cycloalkyl-C₁-C₆-alkyl.

A compound of formula I wherein R⁷ is 5- to 6-membered heteroaromatic ring system which may contain 1 to 4 heteroatoms selected from oxygen, nitrogen and sulfur.

A compound of formula I wherein x is 0.

A compound of formula I wherein x is 1.

A compound of formula I wherein y is 0.

3-pyridyl derivative compounds of formula I wherein R² and/or R⁷ is selected from a ring system Q of table A.

A compound of formula I wherein R² is selected from a phenyl ring P of table B, especially those phenyl rings P wherein the 4-position or the 3- and 4-position is/are substituted each independently with fluoro, chloro, bromo, iodo, amino, nitro, methyl, or methoxy.

TABLE A

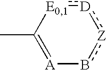

| No. | A | B | Z | D | E |
|---|---|---|---|---|---|
| Q.1 | ═N— | —C(R_{q})═ | ═C(R_{q})— | —C(R_{q})═ | ═C(R_{q})— |
| Q.2 | ═C(R_{q})— | —N═ | ═C(R_{q})— | —C(R_{q})═ | ═C(R_{q})— |
| Q.3 | ═C(R_{q})— | —C(R_{q})═ | ═N— | —C(R_{q})═ | ═C(R_{q})— |
| Q.4 | ═C(R_{q})— | —C(R_{q})═ | ═C(R_{q})— | —S— | — |
| Q.5 | ═C(R_{q})— | —S— | —C(R_{q})═ | ═C(R_{q})— | — |
| Q.6 | ═C(R_{q})— | —C(R_{q})═ | ═C(R_{q})— | —O— | — |
| Q.7 | ═C(R_{q})— | —O— | —C(R_{q})═ | ═C(R_{q})— | — |
| Q.8 | ═N— | —C(R_{q})═ | ═C(R_{q})— | —S— | — |
| Q.9 | ═N— | —C(R_{q})═ | ═C(R_{q})— | —O— | — |
| Q.10 | ═C(R_{q})— | —N═ | ═C(R_{q})— | —S— | — |
| Q.11 | ═C(R_{q})— | —N═ | ═C(R_{q})— | —O— | — |
| Q.12 | ═C(R_{q})— | —S— | —C(R_{q})═ | ═N— | — |
| Q.13 | ═C(R_{q})— | —O— | —C(R_{q})═ | ═N— | — |
| Q.14 | ═N— | —S— | —C(R_{q})═ | ═C(R_{q})— | — |
| Q.15 | ═N— | —O— | —C(R_{q})═ | ═C(R_{q})— | — |
| Q.16 | ═N— | —N(R_{q})— | —C(R_{q})═ | ═C(R_{q})— | — |
| Q.17 | ═N— | —N═ | ═C(R_{q})— | —N(R_{q})— | — |
| Q.18 | ═N— | —N═ | ═C(R_{q})— | —S— | — |
| Q.19 | ═N— | —N═ | ═C(R_{q})— | —O— | — |
| Q.20 | ═N— | —O— | —C(R_{q})═ | ═N— | — |
| Q.21 | ═N— | —N═ | ═C(R_{q})— | —C(R_{q})═ | ═C(R_{q})— |
| Q.22 | ═C(R_{q})— | —N═ | ═N— | —C(R_{q})═ | ═C(R_{q})— |
| Q.23 | ═N— | —C(R_{q})═ | ═N— | —C(R_{q})═ | ═C(R_{q})— |
| Q.24 | ═N— | —C(R_{q})═ | ═C(R_{q})— | —N═ | ═C(R_{q})— |
| Q.25 | ═N— | —C(R_{q})═ | ═C(R_{q})— | —C(R_{q})═ | ═N— |
| Q.26 | ═C(R_{q})— | —N═ | ═C(R_{q})— | —N═ | ═C(R_{q})— |
| Q.27 | ═C(R_{q})— | —S— | —N═ | ═C(R_{q})— | — |
| Q.28 | ═C(R_{q})— | —O— | —N═ | ═C(R_{q})— | — |
| Q.29 | ═C(R_{q})— | —C(R_{q})═ | ═N— | —S— | — |
| Q.30 | ═C(R_{q})— | —C(R_{q})═ | ═N— | —O— | — |
| Q.31 | ═C(R_{q})— | —N(R_{q})— | —N═ | ═C(R_{q})— | — |

In the rings Q.1 to Q.31, R_{q} may be the same or different and is selected from the group: hydrogen, fluoro, chloro, bromo, methyl, methoxy, trifluoromethyl, trifluoromethoxy, methylthio, cyano, amino and methylsulfonyl. Preferably, the rings Q.1 and Q.25 carry 0, 1, 2, or 3 substituents R_{q} other than hydrogen, preferably at the positions B, Z, and/or D. Also, preferably, the rings Q.2, Q.3, Q.4, Q.6, Q.8, Q.9, Q.16, Q.21, Q.23, Q.24 carry 0, 1 or 2 substituents R_{q} other than hydrogen, preferably at the positions: Z and D (in the case of the rings Q.2, Q.21), B and D (in the case of the rings Q.3, Q.23), or B and Z (in the case of the rings Q.4, Q.6., Q.8, Q.9, Q.16, Q.24). Also, preferably, the rings Q.5, Q.7, Q.10, Q.11, Q.12, Q.13, Q.14, Q.15, Q.17, Q.18, Q.19, Q.20, Q.22, Q.25, Q.29, Q.30 and Q.31 carry 0 or 1 substituents R_{q} other than hydrogen, preferably at the positions Z (in the case of the rings Q.5, Q.7, Q.10, Q.11, Q.12, Q.13, Q.14, Q.15, Q.17, Q.18, Q.19, Q.20, Q.26), B (in the case of the rings Q.29, Q.30, Q.31), or D (for Q.22). Q.27 and Q.28 preferably are substituted with hydrogen.

A compound of formula I wherein R² and/or R⁷ are independently selected from a group Q*, wherein

Q* is 2-pyridyl, 3-pyridyl, 4-pyridyl, 2-thiophenyl, 3-thiophenyl, 2-furanyl, 3-furanyl, 3-isothiazolyl, 4-isothiazolyl, 5-isothiazolyl, 3-isoxazolyl, 4-isoxazolyl, 5-isoxazolyl, 2-oxazolyl, 4-oxazolyl, 5-oxazolyl, 2-thiazolyl, 4-thiazolyl, 5-thiazolyl.

With respect to their use, particular preference is given to the compounds I wherein R⁷ is selected from methyl, ethyl, propyl, isopropyl, butyl, isobutyl, sec.-butyl, tert.-butyl, pentyl, vinyl, propargyl, cyclopropyl, cyclobutyl, cyclopentyl, cyclohexyl, cyclopropylmethyl, methoxymethyl, ethoxymethyl, chloromethyl, cyanomethyl, methylthiomethyl, ethylthiomethyl, benzyl, 2-tetrahydrofuranyl, 3-tetrahydrofuranyl, 5-oxo-tetrahydrofuran-2-yl, 5-oxo-tetrahydrofuran-3-yl, 2-tetrahydrothiophenyl, 3-tetrahydrothiophenyl, 3-tetrahydrofuranyl, 4-tetrahydrofuranyl, 3-tetrahydrothiophenyl, 4-tetrahydrothiophenyl, 4-piperidinyl, 1-methyl-piperidin-1-yl, benzyl, a phenyl group P compiled in table B below, and a group Q*.

TABLE B

| | |
|---|---|
| | P |
| 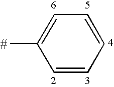 | |

| No. | Substituent(s) | No. | Substituent(s) |
|---|---|---|---|
| P.1 | — | P.2 | 3-F |
| P.3 | 3-Cl | P.4 | 3-Br |
| P.5 | 3-I | P.6 | 3-CH₃ |
| P.7 | 3-NO₂ | P.8 | 3-NH₂ |
| P.9 | 3-CH₂CH₃ | P.10 | 3-CH(CH₃)₂ |
| P.11 | 3-CH═CH₂ | P.12 | 3-CCH |
| P.13 | 3-CH₂CCH | P.14 | 3-CH₂CHCH₂ |
| P.15 | 3-CCCH₃ | P.16 | 3-CCCH₂CH₃ |
| P.17 | 3-CC(CH₂)₂CH₃ | P.18 | 3-CC(CH₂)₃CH₃ |
| P.19 | 3-CN | P.20 | 3-OCH₃ |
| P.21 | 3-OCH₂CH₃ | P.22 | 3-OCH₂CCH |
| P.23 | 3-OCH₂CHCH₂ | P.24 | 3-SCH₃ |
| P.25 | 3-OCF₃ | P.26 | 3-S(═O)₂NH₂ |
| P.27 | 3-S(═O)CH₃ | P.28 | 3-S(═O)₂CH₃ |
| P.29 | 3-S(═O)₂CF₃ | P.30 | 3-SCF₃ |
| P.31 | 3-S(═O)CF₃ | P.32 | 3-C₆H₅ |
| P.33 | 3-COOH | P.34 | 3-COOCH₃ |
| P.35 | 3-COOCH₂CH₃ | P.36 | 3-COOCHCH₂ |
| P.37 | 3-COOCCH | P.38 | 3-COOC(CH₃)₃ |
| P.39 | 3-CONH₂ | P.40 | 3-CONHCH₃ |
| P.41 | 3-CON(CH₃)₂ | P.42 | 3-CF₃ |
| P.43 | 3-CH═NOH | P.44 | 3-CH═NOCH₃ |
| P.45 | 3-NHC(═O)OCH₃ | P.46 | 3-NHC(═O)NCH₃ |
| P.47 | 3-NHC(═O)CH₃ | P.48 | 4-F |
| P.49 | 4-Cl | P.50 | 4-Br |
| P.51 | 4-I | P.52 | 4-CH₃ |
| P.53 | 4-NO₂ | P.54 | 4-NH₂ |
| P.55 | 4-CH₂CH₃ | P.56 | 4-CH(CH₃)₂ |
| P.57 | 4-CH═CH₂ | P.58 | 4-CCH |
| P.59 | 4-CH₂CCH | P.60 | 4-CH₂CHCH₂ |
| P.61 | 4-CCCH₃ | P.62 | 4-CCCH₂CH₃ |
| P.63 | 4-CC(CH₂)₂CH₃ | P.64 | 4-CC(CH₂)₃CH₃ |
| P.65 | 4-CN | P.66 | 4-OCH₃ |
| P.67 | 4-OCH₂CH₃ | P.68 | 4-OCH₂CCH |
| P.69 | 4-OCH₂CHCH₂ | P.70 | 4-SCH₃ |
| P.71 | 4-OCF₃ | P.72 | 4-S(═O)₂NH₂ |
| P.73 | 4-S(═O)CH₃ | P.74 | 4-S(═O)₂CH₃ |
| P.75 | 4-S(═O)₂CF₃ | P.76 | 4-SCF₃ |
| P.77 | 4-S(═O)CF₃ | P.78 | 4-C₆H₅ |
| P.79 | 4-COOH | P.80 | 4-COOCH₃ |
| P.81 | 4-COOCH₂CH₃ | P.82 | 4-COOCHCH₂ |
| P.83 | 4-COOCCH | P.84 | 4-COOC(CH₃)₃ |
| P.85 | 4-CONH₂ | P.86 | 4-CONHCH₃ |
| P.87 | 4-CON(CH₃)₂ | P.88 | 4-CF₃ |
| P.89 | 4-CH═NOH | P.90 | 4-CH═NOCH₃ |
| P.91 | 4-NHC(═O)OCH₃ | P.92 | 4-NHC(═O)NCH₃ |
| P.93 | 4-NHC(═O)CH₃ | P.94 | 4-SCH₂CH₃ |
| P.95 | 3-Cl, 4-F | P.96 | 3-Cl, 4-Cl |
| P.97 | 3-Cl, 4-CH₃ | P.98 | 3-Cl, 4-CF₃ |
| P.99 | 3-Cl, 4-OCH₃ | P.100 | 3-Cl, 4-OCF₃ |
| P.101 | 3-Cl, 4-NH₂ | P.102 | 3-Cl, 4-NO₂ |
| P.103 | 3-Cl, 4-SCH₃ | P.104 | 3-F, 4-F |
| P.105 | 3-F, 4-Cl | P.106 | 3-F, 4-CH₃ |
| P.107 | 3-F, 4-CF₃ | P.108 | 3-F, 4-OCH₃ |
| P.109 | 3-F, 4-OCF₃ | P.110 | 3-F, 4-SCH₃ |
| P.111 | 3-F, 4-NH₂ | P.112 | 3-F, 4-NO₂ |
| P.113 | 3-Br, 4-F | P.114 | 3-Br, 4-Cl |
| P.115 | 3-Br, 4-CH₃ | P.116 | 3-Br, 4-CF₃ |
| P.117 | 3-Br, 4-OCH₃ | P.118 | 3-Br, 4-OCF₃ |
| P.119 | 3-Br, 4-NH₂ | P.120 | 3-Br, 4-NO₂ |
| P.121 | 3-Br, 4-SCH₃ | P.122 | 3-CH₃, 4-F |
| P.123 | 3-CH₃, 4-Cl | P.124 | 3-CH₃, 4-CH₃ |
| P.125 | 3-CH₃, 4-CF₃ | P.126 | 3-CH₃, 4-OCH₃ |
| P.127 | 3-CH₃, 4-NH₂ | P.128 | 3-CH₃, 4-NO₂ |
| P.129 | 3-CH₃, 4-OCF₃ | P.130 | 3-CH₃, 4-SCH₃ |
| P.131 | 3-CH₂CH₃, 4-F | P.132 | 3-CH₂CH₃, 4-Cl |
| P.133 | 3-CH₂CH₃, 4-CH₃ | P.134 | 3-CH₂CH₃, 4-CF₃ |
| P.135 | 3-CH₂CH₃, 4-OCH₃ | P.136 | 3-CH₂CH₃, 4-OCF₃ |
| P.137 | 3-CH₂CH₃, 4-NH₂ | P.138 | 3-CH₂CH₃, 4-NO₂ |
| P.139 | 3-CH₂CH₃, 4-SCH₃ | P.140 | 3-CH(CH₃)₂, 4-F |
| P.141 | 3-CH(CH₃)₂, 4-Cl | P.142 | 3-CH(CH₃)₂, 4-CH₃ |
| P.143 | 3-CH(CH₃)₂, 4-CF₃ | P.144 | 3-CH(CH₃)₂, 4-OCH₃ |
| P.145 | 3-CH(CH₃)₂, 4-NH₂ | P.146 | 3-CH(CH₃)₂, 4-NO₂ |
| P.147 | 3-CH(CH₃)₂, 4-OCF₃ | P.148 | 3-CH(CH₃)₂, 4-SCH₃ |
| P.149 | 3-CHCH₂, 4-F | P.150 | 3-CHCH₂, 4-Cl |
| P.151 | 3-CHCH₂, 4-CH₃ | P.152 | 3-CHCH₂, 4-CF₃ |
| P.153 | 3-CHCH₂, 4-NH₂ | P.154 | 3-CHCH₂, 4-NO₂ |
| P.155 | 3-CHCH₂, 4-OCH₃ | P.156 | 3-CHCH₂, 4-OCF₃ |
| P.157 | 3-CHCH₂, 4-SCH₃ | P.158 | 3-CCH, 4-F |
| P.159 | 3-CCH, 4-Cl | P.160 | 3-CCH, 4-CH₃ |
| P.161 | 3-CCH, 4-CF₃ | P.162 | 3-CCH, 4-OCH₃ |
| P.163 | 3-CCH, 4-NH₂ | P.164 | 3-CCH, 4-NO₂ |
| P.165 | 3-CCH, 4-OCF₃ | P.166 | 3-CCH, 4-SCH₃ |
| P.167 | 3-CC—CH₃, 4-F | P.168 | 3-CC—CH₃, 4-Cl |
| P.169 | 3-CC—CH₃, 4-CH₃ | P.170 | 3-CC—CH₃, 4-CF₃ |
| P.171 | 3-CC—CH₃, 4-OCH₃ | P.172 | 3-CC—CH₃, 4-OCF₃ |
| P.173 | 3-CC—CH₃, 4-NH₂ | P.174 | 3-CC—CH₃, 4-NO₂ |
| P.175 | 3-CC—CH₃, 4-SCH₃ | P.176 | 3-CC—CH₂CH₃, 4-F |
| P.177 | 3-CC—CH₂CH₃, 4-Cl | P.178 | 3-CC—CH₂CH₃, 4-CH₃ |
| P.179 | 3-CC—CH₂CH₃, 4-CF₃ | P.180 | 3-CC—CH₂CH₃, 4-OCH₃ |
| P.181 | 3-CC—CH₂CH₃, 4-NH₃ | P.182 | 3-CC—CH₂CH₃, 4-NO₂ |
| P.183 | 3-CC—CH₂CH₃, 4-OCF₃ | P.184 | 3-CC—CH₂CH₃, 4-SCH₃ |
| P.185 | 3-CC—(CH₂)₂CH₃, 4-F | P.186 | 3-CC—(CH₂)₂CH₃, 4-Cl |
| P.187 | 3-CC—(CH₂)₂CH₃, 4-CH₃ | P.188 | 3-CC—(CH₂)₂CH₃, 4-CF₃ |
| P.189 | 3-CC—(CH₂)₂CH₃, 4-NH₂ | P.190 | 3-CC—(CH₂)₂CH₃, 4-NO₂ |
| P.191 | 3-CC—(CH₂)₂CH₃, | P.192 | 3-CC—(CH₂)₂CH₃, 4-OCF₃ |
| | 4-OCH₃ | | |
| P.193 | 3-CC—(CH₂)₂CH₃, | P.194 | 3-CC—(CH₂)₃CH₃, 4-F |
| | 4-SCH₃ | | |
| P.195 | 3-CC—(CH₂)₃CH₃, 4-Cl | P.196 | 3-CC—(CH₂)₃CH₃, 4-CH₃ |
| P.197 | 3-CC—(CH₂)₃CH₃, 4-NH₂ | P.198 | 3-CC—(CH₂)₃CH₃, 4-NO₂ |
| P.199 | 3-CC—(CH₂)₃CH₃, 4-CF₃ | P.200 | 3-CC—(CH₂)₃CH₃, 4-OCH₃ |
| P.201 | 3-CC—(CH₂)₃CH₃, | P.202 | 3-CC—(CH₂)₃CH₃, 4-SCH₃ |
| | 4-OCF₃ | | |
| P.203 | 3-CN, 4-F | P.204 | 3-CN, 4-Cl |
| P.205 | 3-CN, 4-CH₃ | P.206 | 3-CN, 4-CF₃ |
| P.207 | 3-CN, 4-OCH₃ | P.208 | 3-CN, 4-OCF₃ |
| P.209 | 3-CN, 4-NH₂ | P.210 | 3-CN, 4-NO₂ |
| P.211 | 3-CN, 4-SCH₃ | P.212 | 3-SCH₃, 4-F |
| P.213 | 3-SCH₃, 4-Cl | P.214 | 3-SCH₃, 4-CH₃ |
| P.215 | 3-SCH₃, 4-CF₃ | P.216 | 3-SCH₃, 4-OCH₃ |
| P.217 | 3-SCH₃, 4-NH₂ | P.218 | 3-SCH₃, 4-NO₂ |
| P.219 | 3-SCH₃, 4-OCF₃ | P.220 | 3-SCH₃, 4-SCH₃ |
| P.221 | 3-S(═O)CH₃, 4-F | P.222 | 3-S(═O)CH₃, 4-Cl |
| P.223 | 3-S(═O)CH₃, 4-CH₃ | P.224 | 3-S(═O)CH₃, 4-CF₃ |
| P.225 | 3-S(═O)CH₃, 4-OCH₃ | P.226 | 3-S(═O)CH₃, 4-OCF₃ |
| P.227 | 3-S(═O)CH₃, 4-NH₂ | P.228 | 3-S(═O)CH₃, 4-NO₂ |
| P.229 | 3-S(═O)CH₃, 4-SCH₃ | P.230 | 3-S(═O)₂ CH₃, 4-F |
| P.231 | 3-S(═O)₂CH₃, 4-Cl | P.232 | 3-S(═O)₂CH₃, 4-CH₃ |
| P.233 | 3-S(═O)₂CH₃, 4-CF₃ | P.234 | 3-S(═O)₂CH₃, 4-OCH₃ |
| P.235 | 3-S(═O)₂CH₃, 4-NH₂ | P.236 | 3-S(═O)₂CH₃, 4-NO₂ |
| P.237 | 3-S(═O)₂CH₃, 4-OCF₃ | P.238 | 3-S(═O)₂CH₃, 4-SCH₃ |
| P.239 | 3-SCF₃, 4-F | P.240 | 3-SCF₃, 4-Cl |
| P.241 | 3-SCF₃, 4-CH₃ | P.242 | 3-SCF₃, 4-CF₃ |
| P.243 | 3-SCF₃, 4-OCH₃ | P.244 | 3-SCF₃, 4-OCF₃ |
| P.245 | 3-SCF₃, 4-NH₂ | P.246 | 3-SCF₃, 4-NO₂ |
| P.247 | 3-SCF₃, 4-SCH₃ | P.248 | 3-S(═O)CF₃, 4-F |
| P.249 | 3-S(═O)CF₃, 4-Cl | P.250 | 3-S(═O)CF₃, 4-CH₃ |
| P.251 | 3-S(═O)CF₃, 4-CF₃ | P.252 | 3-S(═O)CF₃, 4-OCH₃ |
| P.253 | 3-S(═O)CF₃, 4-NH₂ | P.254 | 3-S(═O)CF₃, 4-NO₂ |
| P.255 | 3-S(═O)CF₃, 4-OCF₃ | P.256 | 3-S(═O)CF₃, 4-SCH₃ |
| P.257 | 3-S(═O)₂CF₃, 4-F | P.258 | 3-S(═O)₂CF₃, 4-Cl |
| P.259 | 3-S(═O)₂CF₃, 4-CH₃ | P.260 | 3-S(═O)₂CF₃, 4-CF₃ |
| P.261 | 3-S(═O)₂CF₃, 4-OCH₃ | P.262 | 3-S(═O)₂CF₃, 4-OCF₃ |
| P.263 | 3-S(═O)₂CF₃, 4-NH₂ | P.264 | 3-S(═O)₂CF₃, 4-NO₂ |
| P.265 | 3-S(═O)₂CF₃, 4-SCH₃ | P.266 | 3-OCH₃, 4-F |
| P.267 | 3-OCH₃, 4-Cl | P.268 | 3-OCH₃, 4-CH₃ |
| P.269 | 3-OCH₃, 4-CF₃ | P.270 | 3-OCH₃, 4-OCH₃ |
| P.271 | 3-OCH₃, 4-OCF₃ | P.272 | 3-OCH₃, 4-SCH₃ |
| P.273 | 3-OCH₃, 4-NH₂ | P.274 | 3-OCH₃, 4-NO₂ |
| P.275 | 3-OCH₂CH₃, 4-F | P.276 | 3-OCH₂CH₃, 4-Cl |
| P.277 | 3-OCH₂CH₃, 4-CH₃ | P.278 | 3-OCH₂CH₃, 4-CF₃ |
| P.279 | 3-OCH₂CH₃, 4-OCH₃ | P.280 | 3-OCH₂CH₃, 4-OCF₃ |
| P.281 | 3-OCH₂CH₃, 4-NH₂ | P.282 | 3-OCH₂CH₃, 4-NO₂ |
| P.283 | 3-OCH₂CH₃, 4-SCH₃ | P.284 | 3-OCH₂CHCH₂, 4-F |
| P.285 | 3-OCH₂CHCH₂, 4-Cl | P.286 | 3-OCH₂CHCH₂, 4-CH₃ |
| P.287 | 3-OCH₂CHCH₂, 4-CF₃ | P.288 | 3-OCH₂CHCH₂, 4-OCH₃ |
| P.289 | 3-OCH₂CHCH₂, 4-NH₂ | P.290 | 3-OCH₂CHCH₂, 4-NO₂ |
| P.291 | 3-OCH₂CHCH₂, 4-OCF₃ | P.292 | 3-OCH₂CHCH₂, 4-SCH₃ |
| P.293 | 3-OCH₂CCH, 4-F | P.294 | 3-OCH₂CCH, 4-Cl |
| P.295 | 3-OCH₂CCH, 4-CH₃ | P.296 | 3-OCH₂CCH, 4-CF₃ |
| P.297 | 3-OCH₂CCH, 4-OCH₃ | P.298 | 3-OCH₂CCH, 4-OCF₃ |
| P.299 | 3-OCH₂CCH, 4-NH₂ | P.300 | 3-OCH₂CCH, 4-NO₂ |
| P.301 | 3-OCH₂CCH, 4-SCH₃ | P.302 | 3-C₆H₅, 4-F |
| P.303 | 3-C₆H₅, 4-Cl | P.304 | 3-C₆H₅, 4-CH₃ |
| P.305 | 3-C₆H₅, 4-CF₃ | P.306 | 3-C₆H₅, 4-OCH₃ |
| P.307 | 3-C₆H₅, 4-NH₂ | P.308 | 3-C₆H₅, 4-NO₂ |
| P.309 | 3-C₆H₅, 4-OCF₃ | P.310 | 3-C₆H₅, 4-SCH₃ |
| P.311 | 3-COOH, 4-F | P.312 | 3-COOH, 4-Cl |
| P.313 | 3-COOH, 4-CH₃ | P.314 | 3-COOH, 4-CF₃ |
| P.315 | 3-COOH, 4-OCH₃ | P.316 | 3-COOH, 4-OCF₃ |
| P.317 | 3-COOH, 4-NH₂ | P.318 | 3-COOH, 4-NO₂ |
| P.319 | 3-COOH, 4-SCH₃ | P.320 | 3-COOCH₃, 4-F |
| P.321 | 3-COOCH₃, 4-Cl | P.322 | 3-COOCH₃, 4-CH₃ |
| P.323 | 3-COOCH₃, 4-CF₃ | P.324 | 3-COOCH₃, 4-OCH₃ |
| P.325 | 3-COOCH₃, 4-OCF₃ | P.326 | 3-COOCH₃, 4-SCH₃ |
| P.327 | 3-COOCH₃, 4-NH₂ | P.328 | 3-COOCH₃, 4-NO₂ |
| P.329 | 3-COOCH₂CH₃, 4-F | P.330 | 3-COOCH₂CH₃, 4-Cl |
| P.331 | 3-COOCH₂CH₃, 4-CH₃ | P.332 | 3-COOCH₂CH₃, 4-CF₃ |
| P.333 | 3-COOCH₂CH₃, 4-OCH₃ | P.334 | 3-COOCH₂CH₃, 4-OCF₃ |
| P.335 | 3-COOCH₂CH₃, 4-NH₂ | P.336 | 3-COOCH₂CH₃, 4-NO₂ |
| P.337 | 3-COOCH₂CH₃, 4-SCH₃ | P.338 | 3-COOCHCH₂, 4-F |
| P.339 | 3-COOCH₂CH₃, 4-Cl | P.340 | 3-COOCHCH₂, 4-CH₃ |
| P.341 | 3-COOCH₂CH₃, 4-CF₃ | P.342 | 3-COOCHCH₂, 4-OCH₃ |
| P.343 | 3-COOCH₂CH₃, 4-NH₂ | P.344 | 3-COOCHCH₂, 4-NO₂ |
| P.345 | 3-COOCH₂CH₃, 4-OCF₃ | P.346 | 3-COOCHCH₂, 4-SCH₃ |
| P.347 | 3-COOCCH, 4-F | P.348 | 3-COOCCH, 4-Cl |
| P.349 | 3-COOCCH, 4-CH₃ | P.350 | 3-COOCCH, 4-CF₃ |
| P.351 | 3-COOCCH, 4-OCH₃ | P.352 | 3-COOCCH, 4-OCF₃ |
| P.353 | 3-COOCCH, 4-NH₂ | P.354 | 3-COOCCH, 4-NO₂ |
| P.355 | 3-COOCCH, 4-SCH₃ | P.356 | 3-COOC(CH₃)₃, 4-F |
| P.357 | 3-COOC(CH₃)₃, 4-Cl | P.358 | 3-COOC(CH₃)₃, 4-CH₃ |
| P.359 | 3-COOC(CH₃)₃, 4-CF₃ | P.360 | 3-COOC(CH₃)₃, 4-OCH₃ |
| P.361 | 3-COOC(CH₃)₃, 4-NH₂ | P.362 | 3-COOC(CH₃)₃, 4-NO₂ |
| P.363 | 3-COOC(CH₃)₃, 4-OCF₃ | P.364 | 3-COOC(CH₃)₃, 4-SCH₃ |
| P.365 | 3-CONH₂, 4-F | P.366 | 3-CONH₂, 4-Cl |
| P.367 | 3-CONH₂, 4-CH₃ | P.368 | 3-CONH₂, 4-CF₃ |
| P.369 | 3-CONH₂, 4-OCH₃ | P.370 | 3-CONH₂, 4-OCF₃ |
| P.371 | 3-CONH₂, 4-NH₂ | P.372 | 3-CONH₂, 4-NO₂ |
| P.373 | 3-CONH₂, 4-SCH₃ | P.374 | 3-CONH(CH₃), 4-F |
| P.375 | 3-CONH(CH₃), 4-Cl | P.376 | 3-CONH(CH₃), 4-CH₃ |
| P.377 | 3-CONH(CH₃), 4-CF₃ | P.378 | 3-CONH(CH₃), 4-OCH₃ |
| P.379 | 3-CONH(CH₃), 4-NH₂ | P.380 | 3-CONH(CH₃), 4-NO₂ |
| P.381 | 3-CONH(CH₃), 4-OCF₃ | P.382 | 3-CONH(CH₃), 4-SCH₃ |
| P.383 | 3-CON(CH₃)₂, 4-F | P.384 | 3-CON(CH₃)₂, 4-Cl |
| P.385 | 3-CON(CH₃)₂, 4-CH₃ | P.386 | 3-CON(CH₃)₂, 4-CF₃ |
| P.387 | 3-CON(CH₃)₂, 4-OCH₃ | P.388 | 3-CON(CH₃)₂, 4-OCF₃ |
| P.389 | 3-CON(CH₃)₂, 4-NH₂ | P.390 | 3-CON(CH₃)₂, 4-NO₂ |
| P.391 | 3-CON(CH₃)₂, 4-SCH₃ | P.392 | 3-CH(═N)OCH₃, 4-Cl |
| P.393 | 3-NH₂, 4-F | P.394 | 3-NH₂, 4-Cl |
| P.395 | 3-NH₂, 4-CH₃ | P.396 | 3-NH₂, 4-CF₃ |
| P.397 | 3-NH₂, 4-OCH₃ | P.398 | 3-NH₂, 4-OCF₃ |
| P.399 | 3-NH₂, 4-NH₂ | P.400 | 3-NH₂, 4-NO₂ |
| P.401 | 3-NH₂, 4-SCH₃ | P.402 | 3-NO₂, 4-F |
| P.403 | 3-NO₂, 4-Cl | P.404 | 3-NO₂, 4-CH₃ |
| P.405 | 3-NO₂, 4-CF₃ | P.406 | 3-NO₂, 4-OCH₃ |
| P.407 | 3-NO₂, 4-NH₂ | P.408 | 3-NO₂, 4-NO₂ |
| P.409 | 3-NO₂, 4-OCF₃ | P.410 | 3-NO₂, 4-SCH₃ |
| P.411 | 3-Cl, 5-F | P.412 | 3-Cl, 5-Cl |
| P.413 | 3-Cl, 5-CH₃ | P.414 | 3-Cl, 5-CF₃ |
| P.415 | 3-Cl, 5-OCH₃ | P.416 | 3-Cl, 5-OCF₃ |
| P.417 | 3-Cl, 5-NH₂ | P.418 | 3-Cl, 5-NO₂ |
| P.419 | 3-Cl, 5-SCH₃ | P.420 | 3-F, 5-F |
| P.421 | 3-F, 5-Cl | P.422 | 3-F, 5-CH₃ |
| P.423 | 3-F, 5-CF₃ | P.424 | 3-F, 5-OCH₃ |
| P.425 | 3-F, 5-OCF₃ | P.426 | 3-F, 5-SCH₃ |
| P.427 | 3-F, 5-NH₂ | P.428 | 3-F, 5-NO₂ |
| P.429 | 3-Br, 5-F | P.430 | 3-Br, 5-Cl |
| P.431 | 3-Br, 5-CH₃ | P.432 | 3-Br, 5-CF₃ |
| P.433 | 3-Br, 5-OCH₃ | P.434 | 3-Br, 5-OCF₃ |
| P.435 | 3-Br, 5-NH₂ | P.436 | 3-Br, 5-NO₂ |
| P.437 | 3-Br, 5-SCH₃ | P.438 | 3-CH₃, 5-F |
| P.439 | 3-CH₃, 5-Cl | P.440 | 3-CH₃, 5-CH₃ |
| P.441 | 3-CH₃, 5-CF₃ | P.442 | 3-CH₃, 5-OCH₃ |
| P.443 | 3-CH₃, 5-NH₂ | P.444 | 3-CH₃, 5-NO₂ |
| P.445 | 3-CH₃, 5-OCF₃ | P.446 | 3-CH₃, 5-SCH₃ |
| P.447 | 3-CH₂CH₃, 5-F | P.448 | 3-CH₂CH₃, 5-Cl |
| P.449 | 3-CH₂CH₃, 5-CH₃ | P.450 | 3-CH₂CH₃, 5-CF₃ |
| P.451 | 3-CH₂CH₃, 5-OCH₃ | P.452 | 3-CH₂CH₃, 5-OCF₃ |
| P.453 | 3-CH₂CH₃, 5-NH₂ | P.454 | 3-CH₂CH₃, 5-NO₂ |
| P.455 | 3-CH₂CH₃, 5-SCH₃ | P.456 | 3-CH(CH₃)₂, 5-F |
| P.457 | 3-CH(CH₃)₂, 5-Cl | P.458 | 3-CH(CH₃)₂, 5-CH₃ |
| P.459 | 3-CH(CH₃)₂, 5-CF₃ | P.460 | 3-CH(CH₃)₂, 5-OCH₃ |
| P.461 | 3-CH(CH₃)₂, 5-NH₂ | P.462 | 3-CH(CH₃)₂, 5-NO₂ |
| P.463 | 3-CH(CH₃)₂, 5-OCF₃ | P.464 | 3-CH(CH₃)₂, 5-SCH₃ |
| P.465 | 3-CHCH₂, 5-F | P.466 | 3-CHCH₂, 5-Cl |
| P.467 | 3-CHCH₂, 5-CH₃ | P.468 | 3-CHCH₂, 5-CF₃ |
| P.469 | 3-CHCH₂, 5-NH₂ | P.470 | 3-CHCH₂, 5-NO₂ |
| P.471 | 3-CHCH₂, 5-OCH₃ | P.472 | 3-CHCH₂, 5-OCF₃ |
| P.473 | 3-CHCH₂, 5-SCH₃ | P.474 | 3-CCH, 5-F |
| P.475 | 3-CCH, 5-Cl | P.476 | 3-CCH, 5-CH₃ |
| P.477 | 3-CCH, 5-CF₃ | P.478 | 3-CCH, 5-OCH₃ |
| P.479 | 3-CCH, 5-NH₂ | P.480 | 3-CCH, 5-NO₂ |
| P.481 | 3-CCH, 5-OCF₃ | P.482 | 3-CCH, 5-SCH₃ |
| P.483 | 3-CC—CH₃, 5-F | P.484 | 3-CC—CH₃, 5-Cl |
| P.485 | 3-CC—CH₃, 5-CH₃ | P.486 | 3-CC—CH₃, 5-CF₃ |
| P.487 | 3-CC—CH₃, 5-OCH₃ | P.488 | 3-CC—CH₃, 5-OCF₃ |
| P.489 | 3-CC—CH₃, 5-NH₂ | P.490 | 3-CC—CH₃, 5-NO₂ |
| P.491 | 3-CC—CH₃, 5-SCH₃ | P.492 | 3-CC—CH₂CH₃, 5-F |
| P.493 | 3-CC—CH₂CH₃, 5-Cl | P.494 | 3-CC—CH₂CH₃, 5-CH₃ |
| P.495 | 3-CC—CH₂CH₃, 5-CF₃ | P.496 | 3-CC—CH₂CH₃, 5-OCH₃ |
| P.497 | 3-CC—CH₂CH₃, 5-NH₂ | P.498 | 3-CC—CH₂CH₃, 5-NO₂ |
| P.499 | 3-CC—CH₂CH₃, 5-OCF₃ | P.500 | 3-CC—CH₂CH₃, 5-SCH₃ |
| P.501 | 3-CC—(CH₂)₂CH₃, 5-F | P.502 | 3-CC—(CH₂)₂CH₃, 5-Cl |
| P.503 | 3-CC—(CH₂)₂CH₃, 5-CH₃ | P.504 | 3-CC—(CH₂)₂CH₃, 5-CF₃ |
| P.505 | 3-CC—(CH₂)₂CH₃, 5-NH₂ | P.506 | 3-CC—(CH₂)₂CH₃, 5-NO₂ |
| P.507 | 3-CC—(CH₂)₂CH₃, | P.508 | 3-CC—(CH₂)₂CH₃, 5-OCF₃ |
| | 5-OCH₃ | | |
| P.509 | 3-CC—(CH₂)₂CH₃, | P.510 | 3-CC—(CH₂)₃CH₃, 5-F |
| | 5-SCH₃ | | |
| P.511 | 3-CC—(CH₂)₃CH₃, 5-Cl | P.512 | 3-CC—(CH₂)₃CH₃, 5-CH₃ |
| P.513 | 3-CC—(CH₂)₃CH₃, 5-NH₂ | P.514 | 3-CC—(CH₂)₃CH₃, 5-NO₂ |
| P.515 | 3-CC—(CH₂)₃CH₃, 5-CF₃ | P.516 | 3-CC—(CH₂)₃CH₃, 5-OCH₃ |
| P.517 | 3-CC—(CH₂)₃CH₃, | P.518 | 3-CC—(CH₂)₃CH₃, 5-SCH₃ |
| | 5-OCF₃ | | |
| P.519 | 3-CN, 5-F | P.520 | 3-CN, 5-Cl |
| P.521 | 3-CN, 5-CH₃ | P.522 | 3-CN, 5-CF₃ |
| P.523 | 3-CN, 5-OCH₃ | P.524 | 3-CN, 5-OCF₃ |
| P.525 | 3-CN, 5-NH₂ | P.526 | 3-CN, 5-NO₂ |
| P.527 | 3-CN, 5-SCH₃ | P.528 | 3-SCH₃, 5-F |
| P.529 | 3-SCH₃, 5-Cl | P.530 | 3-SCH₃, 5-CH₃ |
| P.531 | 3-SCH₃, 5-CF₃ | P.532 | 3-SCH₃, 5-OCH₃ |
| P.533 | 3-SCH₃, 5-NH₂ | P.534 | 3-SCH₃, 5-NO₂ |
| P.535 | 3-SCH₃, 5-OCF₃ | P.536 | 3-SCH₃, 5-SCH₃ |
| P.537 | 3-S(═O)CH₃, 5-F | P.538 | 3-S(═O)CH₃, 5-Cl |
| P.539 | 3-S(═O)CH₃, 5-CH₃ | P.540 | 3-S(═O)CH₃, 5-CF₃ |
| P.541 | 3-S(═O)CH₃, 5-OCH₃ | P.542 | 3-S(═O)CH₃, 5-OCF₃ |
| P.543 | 3-S(═O)CH₃, 5-NH₂ | P.544 | 3-S(═O)CH₃, 5-NO₂ |
| P.545 | 3-S(═O)CH₃, 5-SCH₃ | P.546 | 3-S(═O)₂CH₃, 5-F |
| P.547 | 3-S(═O)₂CH₃, 5-Cl | P.548 | 3-S(═O)₂CH₃, 5-CH₃ |
| P.549 | 3-S(═O)₂CH₃, 5-CF₃ | P.550 | 3-S(═O)₂CH₃, 5-OCH₃ |
| P.551 | 3-S(═O)₂CH₃, 5-NH₂ | P.552 | 3-S(═O)₂CH₃, 5-NO₂ |
| P.553 | 3-S(═O)₂CH₃, 5-OCF₃ | P.554 | 3-S(═O)₂CH₃, 5-SCH₃ |
| P.555 | 3-SCF₃, 5-F | P.556 | 3-SCF₃, 5-Cl |
| P.557 | 3-SCF₃, 5-CH₃ | P.558 | 3-SCF₃, 5-CF₃ |
| P.559 | 3-SCF₃, 5-OCH₃ | P.560 | 3-SCF₃, 5-OCF₃ |
| P.561 | 3-SCF₃, 5-NH₂ | P.562 | 3-SCF₃, 5-NO₂ |
| P.563 | 3-SCF₃, 5-SCH₃ | P.564 | 3-S(═O)CF₃, 5-F |
| P.565 | 3-S(═O)CF₃, 5-Cl | P.566 | 3-S(═O)CF₃, 5-CH₃ |
| P.567 | 3-S(═O)CF₃, 5-CF₃ | P.568 | 3-S(═O)CF₃, 5-OCH₃ |
| P.569 | 3-S(═O)CF₃, 5-NH₂ | P.570 | 3-S(═O)CF₃, 5-NO₂ |
| P.571 | 3-S(═O)CF₃, 5-OCF₃ | P.572 | 3-S(═O)CF₃, 5-SCH₃ |
| P.573 | 3-S(═O)₂CF₃, 5-F | P.574 | 3-S(═O)₂CF₃, 5-Cl |
| P.575 | 3-S(═O)₂CF₃, 5-CH₃ | P.576 | 3-S(═O)₂CF₃, 5-CF₃ |
| P.577 | 3-S(═O)₂CF₃, 5-OCH₃ | P.578 | 3-S(═O)₂CF₃, 5-OCF₃ |
| P.579 | 3-S(═O)₂CF₃, 5-NH₂ | P.580 | 3-S(═O)₂CF₃, 5-NO₂ |
| P.581 | 3-S(═O)₂CF₃, 5-SCH₃ | P.582 | 3-OCH₃, 5-F |
| P.583 | 3-OCH₃, 5-Cl | P.584 | 3-OCH₃, 5-CH₃ |
| P.585 | 3-OCH₃, 5-CF₃ | P.586 | 3-OCH₃, 5-OCH₃ |
| P.587 | 3-OCH₃, 5-OCF₃ | P.588 | 3-OCH₃, 5-SCH₃ |
| P.589 | 3-OCH₃, 5-NH₂ | P.590 | 3-OCH₃, 5-NO₂ |
| P.591 | 3-OCH₂CH₃, 5-F | P.592 | 3-OCH₂CH₃, 5-Cl |
| P.593 | 3-OCH₂CH₃, 5-CH₃ | P.594 | 3-OCH₂CH₃, 5-CF₃ |
| P.595 | 3-OCH₂CH₃, 5-OCH₃ | P.596 | 3-OCH₂CH₃, 5-OCF₃ |
| P.597 | 3-OCH₂CH₃, 5-NH₂ | P.598 | 3-OCH₂CH₃, 5-NO₂ |
| P.599 | 3-OCH₂CH₃, 5-SCH₃ | P.600 | 3-OCH₂CHCH₂, 5-F |
| P.601 | 3-OCH₂CHCH₂, 5-Cl | P.602 | 3-OCH₂CHCH₂, 5-CH₃ |
| P.603 | 3-OCH₂CHCH₂, 5-CF₃ | P.604 | 3-OCH₂CHCH₂, 5-OCH₃ |
| P.605 | 3-OCH₂CHCH₂, 5-NH₂ | P.606 | 3-OCH₂CHCH₂, 5-NO₂ |
| P.607 | 3-OCH₂CHCH₂, 5-OCF₃ | P.608 | 3-OCH₂CHCH₂, 5-SCH₃ |
| P.609 | 3-OCH₂CCH, 5-F | P.610 | 3-OCH₂CCH, 5-Cl |
| P.611 | 3-OCH₂CCH, 5-CH₃ | P.612 | 3-OCH₂CCH, 5-CF₃ |
| P.613 | 3-OCH₂CCH, 5-OCH₃ | P.614 | 3-OCH₂CCH, 5-OCF₃ |
| P.615 | 3-OCH₂CCH, 5-NH₂ | P.616 | 3-OCH₂CCH, 5-NO₂ |
| P.617 | 3-OCH₂CCH, 5-SCH₃ | P.618 | 3-C₆H₅, 5-F |
| P.619 | 3-C₆H₅, 5-Cl | P.620 | 3-C₆H₅, 5-CH₃ |
| P.621 | 3-C₆H₅, 5-CF₃ | P.622 | 3-C₆H₅, 5-OCH₃ |
| P.623 | 3-C₆H₅, 5-NH₂ | P.624 | 3-C₆H₅, 5-NO₂ |
| P.625 | 3-C₆H₅, 5-OCF₃ | P.626 | 3-C₆H₅, 5-SCH₃ |
| P.627 | 3-COOH, 5-F | P.628 | 3-COOH, 5-Cl |
| P.629 | 3-COOH, 5-CH₃ | P.630 | 3-COOH, 5-CF₃ |
| P.631 | 3-COOH, 5-OCH₃ | P.632 | 3-COOH, 5-OCF₃ |
| P.633 | 3-COOH, 5-NH₂ | P.634 | 3-COOH, 5-NO₂ |
| P.635 | 3-COOH, 5-SCH₃ | P.636 | 3-COOCH₃, 5-F |
| P.637 | 3-COOCH₃, 5-Cl | P.638 | 3-COOCH₃, 5-CH₃ |
| P.639 | 3-COOCH₃, 5-CF₃ | P.640 | 3-COOCH₃, 5-OCH₃ |
| P.641 | 3-COOCH₃, 5-OCF₃ | P.642 | 3-COOCH₃, 5-SCH₃ |
| P.643 | 3-COOCH₃, 5-NH₂ | P.644 | 3-COOCH₃, 5-NO₂ |
| P.645 | 3-COOCH₂CH₃, 5-F | P.646 | 3-COOCH₂CH₃, 5-Cl |
| P.647 | 3-COOCH₂CH₃, 5-CH₃ | P.648 | 3-COOCH₂CH₃, 5-CF₃ |
| P.649 | 3-COOCH₂CH₃, 5-OCH₃ | P.650 | 3-COOCH₂CH₃, 5-OCF₃ |
| P.651 | 3-COOCH₂CH₃, 5-NH₂ | P.652 | 3-COOCH₂CH₃, 5-NO₂ |
| P.653 | 3-COOCH₂CH₃, 5-SCH₃ | P.654 | 3-COOCHCH₂, 5-F |
| P.655 | 3-COOCHCH₂, 5-Cl | P.656 | 3-COOCHCH₂, 5-CH₃ |
| P.657 | 3-COOCHCH₂, 5-CF₃ | P.658 | 3-COOCHCH₂, 5-OCH₃ |
| P.659 | 3-COOCHCH₂, 5-NH₂ | P.660 | 3-COOCHCH₂, 5-NO₂ |
| P.661 | 3-COOCHCH₂, 5-OCF₃ | P.662 | 3-COOCHCH₂, 5-SCH₃ |
| P.663 | 3-COOCCH, 5-F | P.664 | 3-COOCCH, 5-Cl |
| P.665 | 3-COOCCH, 5-CH₃ | P.666 | 3-COOCCH, 5-CF₃ |
| P.667 | 3-COOCCH, 5-OCH₃ | P.668 | 3-COOCCH, 5-OCF₃ |
| P.669 | 3-COOCCH, 5-NH₂ | P.670 | 3-COOCCH, 5-NO₂ |
| P.671 | 3-COOCCH, 5-SCH₃ | P.672 | 3-COOC(CH₃)₃, 5-F |
| P.673 | 3-COOC(CH₃)₃, 5-Cl | P.674 | 3-COOC(CH₃)₃, 5-CH₃ |
| P.675 | 3-COOC(CH₃)₃, 5-CF₃ | P.676 | 3-COOC(CH₃)₃, 5-OCH₃ |
| P.677 | 3-COOC(CH₃)₃, 5-NH₂ | P.678 | 3-COOC(CH₃)₃, 5-NO₂ |
| P.679 | 3-COOC(CH₃)₃, 5-OCF₃ | P.680 | 3-COOC(CH₃)₃, 5-SCH₃ |
| P.681 | 3-CONH₂, 5-F | P.682 | 3-CONH₂, 5-Cl |
| P.683 | 3-CONH₂, 5-CH₃ | P.684 | 3-CONH₂, 5-CF₃ |
| P.685 | 3-CONH₂, 5-OCH₃ | P.686 | 3-CONH₂, 5-OCF₃ |
| P.687 | 3-CONH₂, 5-NH₂ | P.688 | 3-CONH₂, 5-NO₂ |
| P.689 | 3-CONH₂, 5-SCH₃ | P.690 | 3-CONH(CH₃), 5-F |
| P.691 | 3-CONH(CH₃), 5-Cl | P.692 | 3-CONH(CH₃), 5-CH₃ |
| P.693 | 3-CONH(CH₃), 5-CF₃ | P.694 | 3-CONH(CH₃), 5-OCH₃ |
| P.695 | 3-CONH(CH₃), 5-NH₂ | P.696 | 3-CONH(CH₃), 5-NO₂ |
| P.697 | 3-CONH(CH₃), 5-OCF₃ | P.698 | 3-CONH(CH₃), 5-SCH₃ |
| P.699 | 3-CON(CH₃)₂, 5-F | P.700 | 3-CON(CH₃)₂, 5-Cl |
| P.701 | 3-CON(CH₃)₂, 5-CH₃ | P.702 | 3-CON(CH₃)₂, 5-CF₃ |
| P.703 | 3-CON(CH₃)₂, 5-OCH₃ | P.704 | 3-CON(CH₃)₂, 5-OCF₃ |
| P.705 | 3-CON(CH₃)₂, 5-NH₂ | P.706 | 3-CON(CH₃)₂, 5-NO₂ |
| P.707 | 3-CON(CH₃)₂, 5-SCH₃ | P.708 | 3-CH(═N)OCH₃, 5-Cl |
| P.709 | 3-NH₂, 5-F | P.710 | 3-NH₂, 5-Cl |
| P.711 | 3-NH₂, 5-CH₃ | P.712 | 3-NH₂, 5-CF₃ |
| P.713 | 3-NH₂, 5-OCH₃ | P.714 | 3-NH₂, 5-OCF₃ |
| P.715 | 3-NH₂, 5-NH₂ | P.716 | 3-NH₂, 5-NO₂ |
| P.717 | 3-NH₂, 5-SCH₃ | P.718 | 3-NO₂, 5-F |
| P.719 | 3-NO₂, 5-Cl | P.720 | 3-NO₂, 5-CH₃ |
| P.721 | 3-NO₂, 5-CF₃ | P.722 | 3-NO₂, 5-OCH₃ |
| P.723 | 3-NO₂, 5-NH₂ | P.724 | 3-NO₂, 5-NO₂ |
| P.725 | 3-NO₂, 5-OCF₃ | P.726 | 3-NO₂, 5-SCH₃ |

With respect to their use, particular preference is given to the compounds I compiled in the tables below. Moreover, the groups mentioned for a substituent in the tables are on their own, independently of the combination in which they are mentioned, a particularly preferred embodiment of the substituent in question.

### Table 1

Compounds of the formula IA wherein x is zero, R¹ is hydrogen and the combination of R² and R⁷ in each case corresponds to a row of Table C.

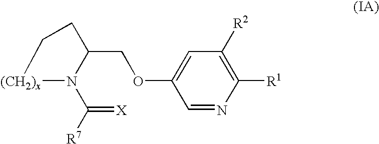

### Table 2

Compounds of the formula IA wherein x is zero, R¹ is fluoro and the combination of R² and R⁷ in each case corresponds to a row of Table C.

### Table 3

Compounds of the formula IA wherein x is zero, R¹ is chloro and the combination of R² and R⁷ in each case corresponds to a row of Table C.

### Table 4

Compounds of the formula IA wherein x is zero, R¹ is bromo and the combination of R² and R⁷ in each case corresponds to a row of Table C.

### Table 5

Compounds of the formula IA wherein x is zero, R¹ is methyl and the combination of R² and R⁷ in each case corresponds to a row of Table C.

### Table 6

Compounds of the formula IA wherein x is zero, R¹ is cyano and the combination of R² and R⁷ in each case corresponds to a row of Table C.

### Table 7

Compounds of the formula IA wherein x is zero, R¹ is methoxy and the combination of R² and R⁷ in each case corresponds to a row of Table C.

### Table 8

Compounds of the formula IA wherein x is zero, R¹ is methylthio and the combination of R² and R⁷ in each case corresponds to a row of Table C.

### Table 9

Compounds of the formula IA wherein x is zero, R¹ is amino and the combination of R² and R⁷ in each case corresponds to a row of Table C.

### Table 10

Compounds of the formula IA wherein x is zero, R¹ is dimethylamino and the combination of R² and R⁷ in each case corresponds to a row of Table C.

### Table 11

Compounds of the formula IA wherein x is 1, R¹ is hydrogen and the combination of R² and R⁷ in each case corresponds to a row of Table C.

### Table 12

Compounds of the formula IA wherein x is 1, R¹ is fluoro and the combination of R² and R⁷ in each case corresponds to a row of Table C.

### Table 13

Compounds of the formula IA wherein x is 1, R¹ is chloro and the combination of R² and R⁷ in each case corresponds to a row of Table C.

### Table 14

Compounds of the formula IA wherein x is 1, R¹ is bromo and the combination of R² and R⁷ in each case corresponds to a row of Table C.

### Table 15

Compounds of the formula IA wherein x is 1, R¹ is methyl and the combination of R² and R⁷ in each case corresponds to a row of Table C.

### Table 16

Compounds of the formula IA wherein x is 1, R¹ is cyano and the combination of R² and R⁷ in each case corresponds to a row of Table C.

### Table 17

Compounds of the formula IA wherein x is 1, R¹ is methoxy and the combination of R² and R⁷ in each case corresponds to a row of Table C.

### Table 18

Compounds of the formula IA wherein x is 1, R¹ is methylthio and the combination of R² and R⁷ in each case corresponds to a row of Table C.

### Table 19

Compounds of the formula IA wherein x is 1, R¹ is amino and the combination of R² and R⁷ in each case corresponds to a row of Table C.

### Table 20

Compounds of the formula IA wherein x is 1, R¹ is dimethylamino and the combination of R² and R⁷ in each case corresponds to a row of Table C.

TABLE C

| No. | R² | R⁷ | No. | R² | R⁷ |
|---|---|---|---|---|---|
| IA-1 | H | CH₃ | IA-2 | F | CH₃ |
| IA-3 | Cl | CH₃ | IA-4 | Br | CH₃ |
| IA-5 | CH₃ | CH₃ | IA-6 | CN | CH₃ |
| IA-7 | CH═CH₂ | CH₃ | IA-8 | CCH | CH₃ |
| IA-9 | 2-pyridyl | CH₃ | IA-10 | 3-pyridyl | CH₃ |
| IA-11 | 4-pyridyl | CH₃ | IA-12 | P.1 | CH₃ |
| IA-13 | P.2 | CH₃ | IA-14 | P.3 | CH₃ |
| IA-15 | P.6 | CH₃ | IA-16 | P.7 | CH₃ |
| IA-17 | P.8 | CH₃ | IA-18 | P.20 | CH₃ |
| IA-19 | P.48 | CH₃ | IA-20 | P.49 | CH₃ |
| IA-21 | P.52 | CH₃ | IA-22 | P.53 | CH₃ |
| IA-23 | P.54 | CH₃ | IA-24 | P.66 | CH₃ |
| IA-25 | P.101 | CH₃ | IA-26 | P.102 | CH₃ |
| IA-27 | P.111 | CH₃ | IA-28 | P.112 | CH₃ |
| IA-29 | P.127 | CH₃ | IA-30 | P.128 | CH₃ |
| IA-31 | P.273 | CH₃ | IA-32 | P.274 | CH₃ |
| IA-33 | P.393 | CH₃ | IA-34 | P.394 | CH₃ |
| IA-35 | P.395 | CH₃ | IA-36 | P.397 | CH₃ |
| IA-37 | P.402 | CH₃ | IA-38 | P.403 | CH₃ |
| IA-39 | P.404 | CH₃ | IA-40 | P.406 | CH₃ |
| IA-41 | H | CH₂CH₃ | IA-42 | F | CH₂CH₃ |
| IA-43 | Cl | CH₂CH₃ | IA-44 | Br | CH₂CH₃ |
| IA-45 | CH₃ | CH₂CH₃ | IA-46 | CN | CH₂CH₃ |
| IA-47 | CH═CH₂ | CH₂CH₃ | IA-48 | CCH | CH₂CH₃ |
| IA-49 | 2-pyridyl | CH₂CH₃ | IA-50 | 3-pyridyl | CH₂CH₃ |
| IA-51 | 4-pyridyl | CH₂CH₃ | IA-52 | P.1 | CH₂CH₃ |
| IA-53 | P.2 | CH₂CH₃ | IA-54 | P.3 | CH₂CH₃ |
| IA-55 | P.6 | CH₂CH₃ | IA-56 | P.7 | CH₂CH₃ |
| IA-57 | P.8 | CH₂CH₃ | IA-58 | P.20 | CH₂CH₃ |
| IA-59 | P.48 | CH₂CH₃ | IA-60 | P.49 | CH₂CH₃ |
| IA-61 | P.52 | CH₂CH₃ | IA-62 | P.53 | CH₂CH₃ |
| IA-63 | P.54 | CH₂CH₃ | IA-64 | P.66 | CH₂CH₃ |
| IA-65 | P.101 | CH₂CH₃ | IA-66 | P.102 | CH₂CH₃ |
| IA-67 | P.111 | CH₂CH₃ | IA-68 | P.112 | CH₂CH₃ |
| IA-69 | P.127 | CH₂CH₃ | IA-70 | P.128 | CH₂CH₃ |
| IA-71 | P.273 | CH₂CH₃ | IA-72 | P.274 | CH₂CH₃ |
| IA-73 | P.393 | CH₂CH₃ | IA-74 | P.394 | CH₂CH₃ |
| IA-75 | P.395 | CH₂CH₃ | IA-76 | P.397 | CH₂CH₃ |
| IA-77 | P.402 | CH₂CH₃ | IA-78 | P.403 | CH₂CH₃ |
| IA-79 | P.404 | CH₂CH₃ | IA-80 | P.406 | CH₂CH₃ |
| IA-81 | H | CH₂CH₂CH₃ | IA-82 | F | CH₂CH₂CH₃ |
| IA-83 | Cl | CH₂CH₂CH₃ | IA-84 | Br | CH₂CH₂CH₃ |
| IA-85 | CH₃ | CH₂CH₂CH₃ | IA-86 | CN | CH₂CH₂CH₃ |
| IA-87 | CH═CH₂ | CH₂CH₂CH₃ | IA-88 | CCH | CH₂CH₂CH₃ |
| IA-89 | 2-pyridyl | CH₂CH₂CH₃ | IA-90 | 3-pyridyl | CH₂CH₂CH₃ |
| IA-91 | 4-pyridyl | CH₂CH₂CH₃ | IA-92 | P.1 | CH₂CH₂CH₃ |
| IA-93 | P.2 | CH₂CH₂CH₃ | IA-94 | P.3 | CH₂CH₂CH₃ |
| IA-95 | P.6 | CH₂CH₂CH₃ | IA-96 | P.7 | CH₂CH₂CH₃ |
| IA-97 | P.8 | CH₂CH₂CH₃ | IA-98 | P.20 | CH₂CH₂CH₃ |
| IA-99 | P.48 | CH₂CH₂CH₃ | IA-100 | P.49 | CH₂CH₂CH₃ |
| IA-101 | P.52 | CH₂CH₂CH₃ | IA-102 | P.53 | CH₂CH₂CH₃ |
| IA-103 | P.54 | CH₂CH₂CH₃ | IA-104 | P.66 | CH₂CH₂CH₃ |
| IA-105 | P.101 | CH₂CH₂CH₃ | IA-106 | P.102 | CH₂CH₂CH₃ |
| IA-107 | P.111 | CH₂CH₂CH₃ | IA-108 | P.112 | CH₂CH₂CH₃ |
| IA-109 | P.127 | CH₂CH₂CH₃ | IA-110 | P.128 | CH₂CH₂CH₃ |
| IA-111 | P.273 | CH₂CH₂CH₃ | IA-112 | P.274 | CH₂CH₂CH₃ |
| IA-113 | P.393 | CH₂CH₂CH₃ | IA-114 | P.394 | CH₂CH₂CH₃ |
| IA-115 | P.395 | CH₂CH₂CH₃ | IA-116 | P.397 | CH₂CH₂CH₃ |
| IA-117 | P.402 | CH₂CH₂CH₃ | IA-118 | P.403 | CH₂CH₂CH₃ |
| IA-119 | P.404 | CH₂CH₂CH₃ | IA-120 | P.406 | CH₂CH₂CH₃ |
| IA-121 | H | (CH₂)₄CH₃ | IA-122 | F | (CH₂)₄CH₃ |
| IA-123 | Cl | (CH₂)₄CH₃ | IA-124 | Br | (CH₂)₄CH₃ |
| IA-125 | CH₃ | (CH₂)₄CH₃ | IA-126 | CN | (CH₂)₄CH₃ |
| IA-127 | CH═CH₂ | (CH₂)₄CH₃ | IA-128 | CCH | (CH₂)₄CH₃ |
| IA-129 | 2-pyridyl | (CH₂)₄CH₃ | IA-130 | 3-pyridyl | (CH₂)₄CH₃ |
| IA-131 | 4-pyridyl | (CH₂)₄CH₃ | IA-132 | P.1 | (CH₂)₄CH₃ |
| IA-133 | P.2 | (CH₂)₄CH₃ | IA-134 | P.3 | (CH₂)₄CH₃ |
| IA-135 | P.6 | (CH₂)₄CH₃ | IA-136 | P.7 | (CH₂)₄CH₃ |
| IA-137 | P.8 | (CH₂)₄CH₃ | IA-138 | P.20 | (CH₂)₄CH₃ |
| IA-139 | P.48 | (CH₂)₄CH₃ | IA-140 | P.49 | (CH₂)₄CH₃ |
| IA-141 | P.52 | (CH₂)₄CH₃ | IA-142 | P.53 | (CH₂)₄CH₃ |
| IA-143 | P.54 | (CH₂)₄CH₃ | IA-144 | P.66 | (CH₂)₄CH₃ |
| IA-145 | P.101 | (CH₂)₄CH₃ | IA-146 | P.102 | (CH₂)₄CH₃ |
| IA-147 | P.111 | (CH₂)₄CH₃ | IA-148 | P.112 | (CH₂)₄CH₃ |
| IA-149 | P.127 | (CH₂)₄CH₃ | IA-150 | P.128 | (CH₂)₄CH₃ |
| IA-151 | P.273 | (CH₂)₄CH₃ | IA-152 | P.274 | (CH₂)₄CH₃ |
| IA-153 | P.393 | (CH₂)₄CH₃ | IA-154 | P.394 | (CH₂)₄CH₃ |
| IA-155 | P.395 | (CH₂)₄CH₃ | IA-156 | P.397 | (CH₂)₄CH₃ |
| IA-157 | P.402 | (CH₂)₄CH₃ | IA-158 | P.403 | (CH₂)₄CH₃ |
| IA-159 | P.404 | (CH₂)₄CH₃ | IA-160 | P.406 | (CH₂)₄CH₃ |
| IA-161 | H | CH(CH₃)₂ | IA-162 | F | CH(CH₃)₂ |
| IA-163 | Cl | CH(CH₃)₂ | IA-164 | Br | CH(CH₃)₂ |
| IA-165 | CH₃ | CH(CH₃)₂ | IA-166 | CN | CH(CH₃)₂ |
| IA-167 | CH═CH₂ | CH(CH₃)₂ | IA-168 | CCH | CH(CH₃)₂ |
| IA-169 | 2-pyridyl | CH(CH₃)₂ | IA-170 | 3-pyridyl | CH(CH₃)₂ |
| IA-171 | 4-pyridyl | CH(CH₃)₂ | IA-172 | P.1 | CH(CH₃)₂ |
| IA-173 | P.2 | CH(CH₃)₂ | IA-174 | P.3 | CH(CH₃)₂ |
| IA-175 | P.6 | CH(CH₃)₂ | IA-176 | P.7 | CH(CH₃)₂ |
| IA-177 | P.8 | CH(CH₃)₂ | IA-178 | P.20 | CH(CH₃)₂ |
| IA-179 | P.48 | CH(CH₃)₂ | IA-180 | P.49 | CH(CH₃)₂ |
| IA-181 | P.52 | CH(CH₃)₂ | IA-182 | P.53 | CH(CH₃)₂ |
| IA-183 | P.54 | CH(CH₃)₂ | IA-184 | P.66 | CH(CH₃)₂ |
| IA-185 | P.101 | CH(CH₃)₂ | IA-186 | P.102 | CH(CH₃)₂ |
| IA-187 | P.111 | CH(CH₃)₂ | IA-188 | P.112 | CH(CH₃)₂ |
| IA-189 | P.127 | CH(CH₃)₂ | IA-190 | P.128 | CH(CH₃)₂ |
| IA-191 | P.273 | CH(CH₃)₂ | IA-192 | P.274 | CH(CH₃)₂ |
| IA-193 | P.393 | CH(CH₃)₂ | IA-194 | P.394 | CH(CH₃)₂ |
| IA-195 | P.395 | CH(CH₃)₂ | IA-196 | P.397 | CH(CH₃)₂ |
| IA-197 | P.402 | CH(CH₃)₂ | IA-198 | P.403 | CH(CH₃)₂ |
| IA-199 | P.404 | CH(CH₃)₂ | IA-200 | P.406 | CH(CH₃)₂ |
| IA-201 | H | CH₂CH(CH₃)₂ | IA-202 | F | CH₂CH(CH₃)₂ |
| IA-203 | Cl | CH₂CH(CH₃)₂ | IA-204 | Br | CH₂CH(CH₃)₂ |
| IA-205 | CH₃ | CH₂CH(CH₃)₂ | IA-206 | CN | CH₂CH(CH₃)₂ |
| IA-207 | CH═CH₂ | CH₂CH(CH₃)₂ | IA-208 | CCH | CH₂CH(CH₃)₂ |
| IA-209 | 2-pyridyl | CH₂CH(CH₃)₂ | IA-210 | 3-pyridyl | CH₂CH(CH₃)₂ |
| IA-211 | 4-pyridyl | CH₂CH(CH₃)₂ | IA-212 | P.1 | CH₂CH(CH₃)₂ |
| IA-213 | P.2 | CH₂CH(CH₃)₂ | IA-214 | P.3 | CH₂CH(CH₃)₂ |
| IA-215 | P.6 | CH₂CH(CH₃)₂ | IA-216 | P.7 | CH₂CH(CH₃)₂ |
| IA-217 | P.8 | CH₂CH(CH₃)₂ | IA-218 | P.20 | CH₂CH(CH₃)₂ |
| IA-219 | P.48 | CH₂CH(CH₃)₂ | IA-220 | P.49 | CH₂CH(CH₃)₂ |
| IA-221 | P.52 | CH₂CH(CH₃)₂ | IA-222 | P.53 | CH₂CH(CH₃)₂ |
| IA-223 | P.54 | CH₂CH(CH₃)₂ | IA-224 | P.66 | CH₂CH(CH₃)₂ |
| IA-225 | P.101 | CH₂CH(CH₃)₂ | IA-226 | P.102 | CH₂CH(CH₃)₂ |
| IA-227 | P.111 | CH₂CH(CH₃)₂ | IA-228 | P.112 | CH₂CH(CH₃)₂ |
| IA-229 | P.127 | CH₂CH(CH₃)₂ | IA-230 | P.128 | CH₂CH(CH₃)₂ |
| IA-231 | P.273 | CH₂CH(CH₃)₂ | IA-232 | P.274 | CH₂CH(CH₃)₂ |
| IA-233 | P.393 | CH₂CH(CH₃)₂ | IA-234 | P.394 | CH₂CH(CH₃)₂ |
| IA-235 | P.395 | CH₂CH(CH₃)₂ | IA-236 | P.397 | CH₂CH(CH₃)₂ |
| IA-237 | P.402 | CH₂CH(CH₃)₂ | IA-238 | P.403 | CH₂CH(CH₃)₂ |
| IA-239 | P.404 | CH₂CH(CH₃)₂ | IA-240 | P.406 | CH₂CH(CH₃)₂ |
| IA-241 | H | C(CH₃)₃ | IA-242 | F | C(CH₃)₃ |
| IA-243 | Cl | C(CH₃)₃ | IA-244 | Br | C(CH₃)₃ |
| IA-245 | CH₃ | C(CH₃)₃ | IA-246 | CN | C(CH₃)₃ |
| IA-247 | CH═CH₂ | C(CH₃)₃ | IA-248 | CCH | C(CH₃)₃ |
| IA-249 | 2-pyridyl | C(CH₃)₃ | IA-250 | 3-pyridyl | C(CH₃)₃ |
| IA-251 | 4-pyridyl | C(CH₃)₃ | IA-252 | P.1 | C(CH₃)₃ |
| IA-253 | P.2 | C(CH₃)₃ | IA-254 | P.3 | C(CH₃)₃ |
| IA-255 | P.6 | C(CH₃)₃ | IA-256 | P.7 | C(CH₃)₃ |
| IA-257 | P.8 | C(CH₃)₃ | IA-258 | P.20 | C(CH₃)₃ |
| IA-259 | P.48 | C(CH₃)₃ | IA-260 | P.49 | C(CH₃)₃ |
| IA-261 | P.52 | C(CH₃)₃ | IA-262 | P.53 | C(CH₃)₃ |
| IA-263 | P.54 | C(CH₃)₃ | IA-264 | P.66 | C(CH₃)₃ |
| IA-265 | P.101 | C(CH₃)₃ | IA-266 | P.102 | C(CH₃)₃ |
| IA-267 | P.111 | C(CH₃)₃ | IA-268 | P.112 | C(CH₃)₃ |
| IA-269 | P.127 | C(CH₃)₃ | IA-270 | P.128 | C(CH₃)₃ |
| IA-271 | P.273 | C(CH₃)₃ | IA-272 | P.274 | C(CH₃)₃ |
| IA-273 | P.393 | C(CH₃)₃ | IA-274 | P.394 | C(CH₃)₃ |
| IA-275 | P.395 | C(CH₃)₃ | IA-276 | P.397 | C(CH₃)₃ |
| IA-277 | P.402 | C(CH₃)₃ | IA-278 | P.403 | C(CH₃)₃ |
| IA-279 | P.404 | C(CH₃)₃ | IA-280 | P.406 | C(CH₃)₃ |
| IA-281 | H | CH═CH₂ | IA-282 | F | CH═CH₂ |
| IA-283 | Cl | CH═CH₂ | IA-284 | Br | CH═CH₂ |
| IA-285 | CH₃ | CH═CH₂ | IA-286 | CN | CH═CH₂ |
| IA-287 | CH═CH₂ | CH═CH₂ | IA-288 | CCH | CH═CH₂ |
| IA-289 | 2-pyridyl | CH═CH₂ | IA-290 | 3-pyridyl | CH═CH₂ |
| IA-291 | 4-pyridyl | CH═CH₂ | IA-292 | P.1 | CH═CH₂ |
| IA-293 | P.2 | CH═CH₂ | IA-294 | P.3 | CH═CH₂ |
| IA-295 | P.6 | CH═CH₂ | IA-296 | P.7 | CH═CH₂ |
| IA-297 | P.8 | CH═CH₂ | IA-298 | P.20 | CH═CH₂ |
| IA-299 | P.48 | CH═CH₂ | IA-300 | P.49 | CH═CH₂ |
| IA-301 | P.52 | CH═CH₂ | IA-302 | P.53 | CH═CH₂ |
| IA-303 | P.54 | CH═CH₂ | IA-304 | P.66 | CH═CH₂ |
| IA-305 | P.101 | CH═CH₂ | IA-306 | P.102 | CH═CH₂ |
| IA-307 | P.111 | CH═CH₂ | IA-308 | P.112 | CH═CH₂ |
| IA-309 | P.127 | CH═CH₂ | IA-310 | P.128 | CH═CH₂ |
| IA-311 | P.273 | CH═CH₂ | IA-312 | P.274 | CH═CH₂ |
| IA-313 | P.393 | CH═CH₂ | IA-314 | P.394 | CH═CH₂ |
| IA-315 | P.395 | CH═CH₂ | IA-316 | P.397 | CH═CH₂ |
| IA-317 | P.402 | CH═CH₂ | IA-318 | P.403 | CH═CH₂ |
| IA-319 | P.404 | CH═CH₂ | IA-320 | P.406 | CH═CH₂ |
| IA-321 | H | CCH | IA-322 | F | CCH |
| IA-323 | Cl | CCH | IA-324 | Br | CCH |
| IA-325 | CH₃ | CCH | IA-326 | CN | CCH |
| IA-327 | CH═CH₂ | CCH | IA-328 | CCH | CCH |
| IA-329 | 2-pyridyl | CCH | IA-330 | 3-pyridyl | CCH |
| IA-331 | 4-pyridyl | CCH | IA-332 | P.1 | CCH |
| IA-333 | P.2 | CCH | IA-334 | P.3 | CCH |
| IA-335 | P.6 | CCH | IA-336 | P.7 | CCH |
| IA-337 | P.8 | CCH | IA-338 | P.20 | CCH |
| IA-339 | P.48 | CCH | IA-340 | P.49 | CCH |
| IA-341 | P.52 | CCH | IA-342 | P.53 | CCH |
| IA-343 | P.54 | CCH | IA-344 | P.66 | CCH |
| IA-345 | P.101 | CCH | IA-346 | P.102 | CCH |
| IA-347 | P.111 | CCH | IA-348 | P.112 | CCH |
| IA-349 | P.127 | CCH | IA-350 | P.128 | CCH |
| IA-351 | P.273 | CCH | IA-352 | P.274 | CCH |
| IA-353 | P.393 | CCH | IA-354 | P.394 | CCH |
| IA-355 | P.395 | CCH | IA-356 | P.397 | CCH |
| IA-357 | P.402 | CCH | IA-358 | P.403 | CCH |
| IA-359 | P.404 | CCH | IA-360 | P.406 | CCH |
| IA-361 | H | CH₂OCH₃ | IA-362 | F | CH₂OCH₃ |
| IA-363 | Cl | CH₂OCH₃ | IA-364 | Br | CH₂OCH₃ |
| IA-365 | CH₃ | CH₂OCH₃ | IA-366 | CN | CH₂OCH₃ |
| IA-367 | CH═CH₂ | CH₂OCH₃ | IA-368 | CCH | CH₂OCH₃ |
| IA-369 | 2-pyridyl | CH₂OCH₃ | IA-370 | 3-pyridyl | CH₂OCH₃ |
| IA-371 | 4-pyridyl | CH₂OCH₃ | IA-372 | P.1 | CH₂OCH₃ |
| IA-373 | P.2 | CH₂OCH₃ | IA-374 | P.3 | CH₂OCH₃ |
| IA-375 | P.6 | CH₂OCH₃ | IA-376 | P.7 | CH₂OCH₃ |
| IA-377 | P.8 | CH₂OCH₃ | IA-378 | P.20 | CH₂OCH₃ |
| IA-379 | P.48 | CH₂OCH₃ | IA-380 | P.49 | CH₂OCH₃ |
| IA-381 | P.52 | CH₂OCH₃ | IA-382 | P.53 | CH₂OCH₃ |
| IA-383 | P.54 | CH₂OCH₃ | IA-384 | P.66 | CH₂OCH₃ |
| IA-385 | P.101 | CH₂OCH₃ | IA-386 | P.102 | CH₂OCH₃ |
| IA-387 | P.111 | CH₂OCH₃ | IA-388 | P.112 | CH₂OCH₃ |
| IA-389 | P.127 | CH₂OCH₃ | IA-390 | P.128 | CH₂OCH₃ |
| IA-391 | P.273 | CH₂OCH₃ | IA-392 | P.274 | CH₂OCH₃ |
| IA-393 | P.393 | CH₂OCH₃ | IA-394 | P.394 | CH₂OCH₃ |
| IA-395 | P.395 | CH₂OCH₃ | IA-396 | P.397 | CH₂OCH₃ |
| IA-397 | P.402 | CH₂OCH₃ | IA-398 | P.403 | CH₂OCH₃ |
| IA-399 | P.404 | CH₂OCH₃ | IA-400 | P.406 | CH₂OCH₃ |
| IA-401 | H | CH₂Cl | IA-402 | F | CH₂Cl |
| IA-403 | Cl | CH₂Cl | IA-404 | Br | CH₂Cl |
| IA-405 | CH₃ | CH₂Cl | IA-406 | CN | CH₂Cl |
| IA-407 | CH═CH₂ | CH₂Cl | IA-408 | CCH | CH₂Cl |
| IA-409 | 2-pyridyl | CH₂Cl | IA-410 | 3-pyridyl | CH₂Cl |
| IA-411 | 4-pyridyl | CH₂Cl | IA-412 | P.1 | CH₂Cl |
| IA-413 | P.2 | CH₂Cl | IA-414 | P.3 | CH₂Cl |
| IA-415 | P.6 | CH₂Cl | IA-416 | P.7 | CH₂Cl |
| IA-417 | P.8 | CH₂Cl | IA-418 | P.20 | CH₂Cl |
| IA-419 | P.48 | CH₂Cl | IA-420 | P.49 | CH₂Cl |
| IA-421 | P.52 | CH₂Cl | IA-422 | P.53 | CH₂Cl |
| IA-423 | P.54 | CH₂Cl | IA-424 | P.66 | CH₂Cl |
| IA-425 | P.101 | CH₂Cl | IA-426 | P.102 | CH₂Cl |
| IA-427 | P.111 | CH₂Cl | IA-428 | P.112 | CH₂Cl |
| IA-429 | P.127 | CH₂Cl | IA-430 | P.128 | CH₂Cl |
| IA-431 | P.273 | CH₂Cl | IA-432 | P.274 | CH₂Cl |
| IA-433 | P.393 | CH₂Cl | IA-434 | P.394 | CH₂Cl |
| IA-435 | P.395 | CH₂Cl | IA-436 | P.397 | CH₂Cl |
| IA-437 | P.402 | CH₂Cl | IA-438 | P.403 | CH₂Cl |
| IA-439 | P.404 | CH₂Cl | IA-440 | P.406 | CH₂Cl |
| IA-441 | H | CH₂SCH₃ | IA-442 | F | CH₂SCH₃ |
| IA-443 | Cl | CH₂SCH₃ | IA-444 | Br | CH₂SCH₃ |
| IA-445 | CH₃ | CH₂SCH₃ | IA-446 | CN | CH₂SCH₃ |
| IA-447 | CH═CH₂ | CH₂SCH₃ | IA-448 | CCH | CH₂SCH₃ |
| IA-449 | 2-pyridyl | CH₂SCH₃ | IA-450 | 3-pyridyl | CH₂SCH₃ |
| IA-451 | 4-pyridyl | CH₂SCH₃ | IA-452 | P.1 | CH₂SCH₃ |
| IA-453 | P.2 | CH₂SCH₃ | IA-454 | P.3 | CH₂SCH₃ |
| IA-455 | P.6 | CH₂SCH₃ | IA-456 | P.7 | CH₂SCH₃ |
| IA-457 | P.8 | CH₂SCH₃ | IA-458 | P.20 | CH₂SCH₃ |
| IA-459 | P.48 | CH₂SCH₃ | IA-460 | P.49 | CH₂SCH₃ |
| IA-461 | P.52 | CH₂SCH₃ | IA-462 | P.53 | CH₂SCH₃ |
| IA-463 | P.54 | CH₂SCH₃ | IA-464 | P.66 | CH₂SCH₃ |
| IA-465 | P.101 | CH₂SCH₃ | IA-466 | P.102 | CH₂SCH₃ |
| IA-467 | P.111 | CH₂SCH₃ | IA-468 | P.112 | CH₂SCH₃ |
| IA-469 | P.127 | CH₂SCH₃ | IA-470 | P.128 | CH₂SCH₃ |
| IA-471 | P.273 | CH₂SCH₃ | IA-472 | P.274 | CH₂SCH₃ |
| IA-473 | P.393 | CH₂SCH₃ | IA-474 | P.394 | CH₂SCH₃ |
| IA-475 | P.395 | CH₂SCH₃ | IA-476 | P.397 | CH₂SCH₃ |
| IA-477 | P.402 | CH₂SCH₃ | IA-478 | P.403 | CH₂SCH₃ |
| IA-479 | P.404 | CH₂SCH₃ | IA-480 | P.406 | CH₂SCH₃ |
| IA-481 | H | CH₂C₆H₅ | IA-482 | F | CH₂C₆H₅ |
| IA-483 | Cl | CH₂C₆H₅ | IA-484 | Br | CH₂C₆H₅ |
| IA-485 | CH₃ | CH₂C₆H₅ | IA-486 | CN | CH₂C₆H₅ |
| IA-487 | CH═CH₂ | CH₂C₆H₅ | IA-488 | CCH | CH₂C₆H₅ |
| IA-489 | 2-pyridyl | CH₂C₆H₅ | IA-490 | 3-pyridyl | CH₂C₆H₅ |
| IA-491 | 4-pyridyl | CH₂C₆H₅ | IA-492 | P.1 | CH₂C₆H₅ |
| IA-493 | P.2 | CH₂C₆H₅ | IA-494 | P.3 | CH₂C₆H₅ |
| IA-495 | P.6 | CH₂C₆H₅ | IA-496 | P.7 | CH₂C₆H₅ |
| IA-497 | P.8 | CH₂C₆H₅ | IA-498 | P.20 | CH₂C₆H₅ |
| IA-499 | P.48 | CH₂C₆H₅ | IA-500 | P.49 | CH₂C₆H₅ |
| IA-501 | P.52 | CH₂C₆H₅ | IA-502 | P.53 | CH₂C₆H₅ |
| IA-503 | P.54 | CH₂C₆H₅ | IA-504 | P.66 | CH₂C₆H₅ |
| IA-505 | P.101 | CH₂C₆H₅ | IA-506 | P.102 | CH₂C₆H₅ |
| IA-507 | P.111 | CH₂C₆H₅ | IA-508 | P.112 | CH₂C₆H₅ |
| IA-509 | P.127 | CH₂C₆H₅ | IA-510 | P.128 | CH₂C₆H₅ |
| IA-511 | P.273 | CH₂C₆H₅ | IA-512 | P.274 | CH₂C₆H₅ |
| IA-513 | P.393 | CH₂C₆H₅ | IA-514 | P.394 | CH₂C₆H₅ |
| IA-515 | P.395 | CH₂C₆H₅ | IA-516 | P.397 | CH₂C₆H₅ |
| IA-517 | P.402 | CH₂C₆H₅ | IA-518 | P.403 | CH₂C₆H₅ |
| IA-519 | P.404 | CH₂C₆H₅ | IA-520 | P.406 | CH₂C₆H₅ |
| IA-521 | H | cyclo-C₃H₅ | IA-522 | F | cyclo-C₃H₅ |
| IA-523 | Cl | cyclo-C₃H₅ | IA-524 | Br | cyclo-C₃H₅ |
| IA-525 | CH₃ | cyclo-C₃H₅ | IA-526 | CN | cyclo-C₃H₅ |
| IA-527 | CH═CH₂ | cyclo-C₃H₅ | IA-528 | CCH | cyclo-C₃H₅ |
| IA-529 | 2-pyridyl | cyclo-C₃H₅ | IA-530 | 3-pyridyl | cyclo-C₃H₅ |
| IA-531 | 4-pyridyl | cyclo-C₃H₅ | IA-532 | P.1 | cyclo-C₃H₅ |
| IA-533 | P.2 | cyclo-C₃H₅ | IA-534 | P.3 | cyclo-C₃H₅ |
| IA-535 | P.6 | cyclo-C₃H₅ | IA-536 | P.7 | cyclo-C₃H₅ |
| IA-537 | P.8 | cyclo-C₃H₅ | IA-538 | P.20 | cyclo-C₃H₅ |
| IA-539 | P.48 | cyclo-C₃H₅ | IA-540 | P.49 | cyclo-C₃H₅ |
| IA-541 | P.52 | cyclo-C₃H₅ | IA-542 | P.53 | cyclo-C₃H₅ |
| IA-543 | P.54 | cyclo-C₃H₅ | IA-544 | P.66 | cyclo-C₃H₅ |
| IA-545 | P.101 | cyclo-C₃H₅ | IA-546 | P.102 | cyclo-C₃H₅ |
| IA-547 | P.111 | cyclo-C₃H₅ | IA-548 | P.112 | cyclo-C₃H₅ |
| IA-549 | P.127 | cyclo-C₃H₅ | IA-550 | P.128 | cyclo-C₃H₅ |
| IA-551 | P.273 | cyclo-C₃H₅ | IA-552 | P.274 | cyclo-C₃H₅ |
| IA-553 | P.393 | cyclo-C₃H₅ | IA-554 | P.394 | cyclo-C₃H₅ |
| IA-555 | P.395 | cyclo-C₃H₅ | IA-556 | P.397 | cyclo-C₃H₅ |
| IA-557 | P.402 | cyclo-C₃H₅ | IA-558 | P.403 | cyclo-C₃H₅ |
| IA-559 | P.404 | cyclo-C₃H₅ | IA-560 | P.406 | cyclo-C₃H₅ |
| IA-561 | H | cyclo-C₆H₁₁ | IA-562 | F | cyclo-C₆H₁₁ |
| IA-563 | Cl | cyclo-C₆H₁₁ | IA-564 | Br | cyclo-C₆H₁₁ |
| IA-565 | CH₃ | cyclo-C₆H₁₁ | IA-566 | CN | cyclo-C₆H₁₁ |
| IA-567 | CH═CH₂ | cyclo-C₆H₁₁ | IA-568 | CCH | cyclo-C₆H₁₁ |
| IA-569 | 2-pyridyl | cyclo-C₆H₁₁ | IA-570 | 3-pyridyl | cyclo-C₆H₁₁ |
| IA-571 | 4-pyridyl | cyclo-C₆H₁₁ | IA-572 | P.1 | cyclo-C₆H₁₁ |
| IA-573 | P.2 | cyclo-C₆H₁₁ | IA-574 | P.3 | cyclo-C₆H₁₁ |
| IA-575 | P.6 | cyclo-C₆H₁₁ | IA-576 | P.7 | cyclo-C₆H₁₁ |
| IA-577 | P.8 | cyclo-C₆H₁₁ | IA-578 | P.20 | cyclo-C₆H₁₁ |
| IA-579 | P.48 | cyclo-C₆H₁₁ | IA-580 | P.49 | cyclo-C₆H₁₁ |
| IA-581 | P.52 | cyclo-C₆H₁₁ | IA-582 | P.53 | cyclo-C₆H₁₁ |
| IA-583 | P.54 | cyclo-C₆H₁₁ | IA-584 | P.66 | cyclo-C₆H₁₁ |
| IA-585 | P.101 | cyclo-C₆H₁₁ | IA-586 | P.102 | cyclo-C₆H₁₁ |
| IA-587 | P.111 | cyclo-C₆H₁₁ | IA-588 | P.112 | cyclo-C₆H₁₁ |
| IA-589 | P.127 | cyclo-C₆H₁₁ | IA-590 | P.128 | cyclo-C₆H₁₁ |
| IA-591 | P.273 | cyclo-C₆H₁₁ | IA-592 | P.274 | cyclo-C₆H₁₁ |
| IA-593 | P.393 | cyclo-C₆H₁₁ | IA-594 | P.394 | cyclo-C₆H₁₁ |
| IA-595 | P.395 | cyclo-C₆H₁₁ | IA-596 | P.397 | cyclo-C₆H₁₁ |
| IA-597 | P.402 | cyclo-C₆H₁₁ | IA-598 | P.403 | cyclo-C₆H₁₁ |
| IA-599 | P.404 | cyclo-C₆H₁₁ | IA-600 | P.406 | cyclo-C₆H₁₁ |
| IA-601 | H | 3-tetrahydrofuranyl | IA-602 | F | 3-tetrahydrofuranyl |
| IA-603 | Cl | 3-tetrahydrofuranyl | IA-604 | Br | 3-tetrahydrofuranyl |
| IA-605 | CH₃ | 3-tetrahydrofuranyl | IA-606 | CN | 3-tetrahydrofuranyl |
| IA-607 | CH═CH₂ | 3-tetrahydrofuranyl | IA-608 | CCH | 3-tetrahydrofuranyl |
| IA-609 | 2-pyridyl | 3-tetrahydrofuranyl | IA-610 | 3-pyridyl | 3-tetrahydrofuranyl |
| IA-611 | 4-pyridyl | 3-tetrahydrofuranyl | IA-612 | P.1 | 3-tetrahydrofuranyl |
| IA-613 | P.2 | 3-tetrahydrofuranyl | IA-614 | P.3 | 3-tetrahydrofuranyl |
| IA-615 | P.6 | 3-tetrahydrofuranyl | IA-616 | P.7 | 3-tetrahydrofuranyl |
| IA-617 | P.8 | 3-tetrahydrofuranyl | IA-618 | P.20 | 3-tetrahydrofuranyl |
| IA-619 | P.48 | 3-tetrahydrofuranyl | IA-620 | P.49 | 3-tetrahydrofuranyl |
| IA-621 | P.52 | 3-tetrahydrofuranyl | IA-622 | P.53 | 3-tetrahydrofuranyl |
| IA-623 | P.54 | 3-tetrahydrofuranyl | IA-624 | P.66 | 3-tetrahydrofuranyl |
| IA-625 | P.101 | 3-tetrahydrofuranyl | IA-626 | P.102 | 3-tetrahydrofuranyl |
| IA-627 | P.111 | 3-tetrahydrofuranyl | IA-628 | P.112 | 3-tetrahydrofuranyl |
| IA-629 | P.127 | 3-tetrahydrofuranyl | IA-630 | P.128 | 3-tetrahydrofuranyl |
| IA-631 | P.273 | 3-tetrahydrofuranyl | IA-632 | P.274 | 3-tetrahydrofuranyl |
| IA-633 | P.393 | 3-tetrahydrofuranyl | IA-634 | P.394 | 3-tetrahydrofuranyl |
| IA-635 | P.395 | 3-tetrahydrofuranyl | IA-636 | P.397 | 3-tetrahydrofuranyl |
| IA-637 | P.402 | 3-tetrahydrofuranyl | IA-638 | P.403 | 3-tetrahydrofuranyl |
| IA-639 | P.404 | 3-tetrahydrofuranyl | IA-640 | P.406 | 3-tetrahydrofuranyl |
| IA-641 | H | 5-oxo- | IA-642 | F | 5-oxo- |
| | | tetrahydrofuran-3-yl | | | tetrahydrofuran-3-yl |
| IA-643 | Cl | 5-oxo- | IA-644 | Br | 5-oxo- |
| | | tetrahydrofuran-3-yl | | | tetrahydrofuran-3-yl |
| IA-645 | CH₃ | 5-oxo- | IA-646 | CN | 5-oxo- |
| | | tetrahydrofuran-3-yl | | | tetrahydrofuran-3-yl |
| IA-647 | CH═CH₂ | 5-oxo- | IA-648 | CCH | 5-oxo- |
| | | tetrahydrofuran-3-yl | | | tetrahydrofuran-3-yl |
| IA-649 | 2-pyridyl | 5-oxo- | IA-650 | 3-pyridyl | 5-oxo- |
| | | tetrahydrofuran-3-yl | | | tetrahydrofuran-3-yl |
| IA-651 | 4-pyridyl | 5-oxo- | IA-652 | P.1 | 5-oxo- |
| | | tetrahydrofuran-3-yl | | | tetrahydrofuran-3-yl |
| IA-653 | P.2 | 5-oxo- | IA-654 | P.3 | 5-oxo- |
| | | tetrahydrofuran-3-yl | | | tetrahydrofuran-3-yl |
| IA-655 | P.6 | 5-oxo- | IA-656 | P.7 | 5-oxo- |
| | | tetrahydrofuran-3-yl | | | tetrahydrofuran-3-yl |
| IA-657 | P.8 | 5-oxo- | IA-658 | P.20 | 5-oxo- |
| | | tetrahydrofuran-3-yl | | | tetrahydrofuran-3-yl |
| IA-659 | P.48 | 5-oxo- | IA-660 | P.49 | 5-oxo- |
| | | tetrahydrofuran-3-yl | | | tetrahydrofuran-3-yl |
| IA-661 | P.52 | 5-oxo- | IA-662 | P.53 | 5-oxo- |
| | | tetrahydrofuran-3-yl | | | tetrahydrofuran-3-yl |
| IA-663 | P.54 | 5-oxo- | IA-664 | P.66 | 5-oxo- |
| | | tetrahydrofuran-3-yl | | | tetrahydrofuran-3-yl |
| IA-665 | P.101 | 5-oxo- | IA-666 | P.102 | 5-oxo- |
| | | tetrahydrofuran-3-yl | | | tetrahydrofuran-3-yl |
| IA-667 | P.111 | 5-oxo- | IA-668 | P.112 | 5-oxo- |
| | | tetrahydrofuran-3-yl | | | tetrahydrofuran-3-yl |
| IA-669 | P.127 | 5-oxo- | IA-670 | P.128 | 5-oxo- |
| | | tetrahydrofuran-3-yl | | | tetrahydrofuran-3-yl |
| IA-671 | P.273 | 5-oxo- | IA-672 | P.274 | 5-oxo- |
| | | tetrahydrofuran-3-yl | | | tetrahydrofuran-3-yl |
| IA-673 | P.393 | 5-oxo- | IA-674 | P.394 | 5-oxo- |
| | | tetrahydrofuran-3-yl | | | tetrahydrofuran-3-yl |
| IA-675 | P.395 | 5-oxo- | IA-676 | P.397 | 5-oxo- |
| | | tetrahydrofuran-3-yl | | | tetrahydrofuran-3-yl |
| IA-677 | P.402 | 5-oxo- | IA-678 | P.403 | 5-oxo- |
| | | tetrahydrofuran-3-yl | | | tetrahydrofuran-3-yl |
| IA-679 | P.404 | 5-oxo- | IA-680 | P.406 | 5-oxo- |
| | | tetrahydrofuran-3-yl | | | tetrahydrofuran-3-yl |
| IA-681 | H | 3-tetrahydropyranyl | IA-682 | F | 3-tetrahydropyranyl |
| IA-683 | Cl | 3-tetrahydropyranyl | IA-684 | Br | 3-tetrahydropyranyl |
| IA-685 | CH₃ | 3-tetrahydropyranyl | IA-686 | CN | 3-tetrahydropyranyl |
| IA-687 | CH═CH₂ | 3-tetrahydropyranyl | IA-688 | CCH | 3-tetrahydropyranyl |
| IA-689 | 2-pyridyl | 3-tetrahydropyranyl | IA-690 | 3-pyridyl | 3-tetrahydropyranyl |
| IA-691 | 4-pyridyl | 3-tetrahydropyranyl | IA-692 | P.1 | 3-tetrahydropyranyl |
| IA-693 | P.2 | 3-tetrahydropyranyl | IA-694 | P.3 | 3-tetrahydropyranyl |
| IA-695 | P.6 | 3-tetrahydropyranyl | IA-696 | P.7 | 3-tetrahydropyranyl |
| IA-697 | P.8 | 3-tetrahydropyranyl | IA-698 | P.20 | 3-tetrahydropyranyl |
| IA-699 | P.48 | 3-tetrahydropyranyl | IA-700 | P.49 | 3-tetrahydropyranyl |
| IA-701 | P.52 | 3-tetrahydropyranyl | IA-702 | P.53 | 3-tetrahydropyranyl |
| IA-703 | P.54 | 3-tetrahydropyranyl | IA-704 | P.66 | 3-tetrahydropyranyl |
| IA-705 | P.101 | 3-tetrahydropyranyl | IA-706 | P.102 | 3-tetrahydropyranyl |
| IA-707 | P.111 | 3-tetrahydropyranyl | IA-708 | P.112 | 3-tetrahydropyranyl |
| IA-709 | P.127 | 3-tetrahydropyranyl | IA-710 | P.128 | 3-tetrahydropyranyl |
| IA-711 | P.273 | 3-tetrahydropyranyl | IA-712 | P.274 | 3-tetrahydropyranyl |
| IA-713 | P.393 | 3-tetrahydropyranyl | IA-714 | P.394 | 3-tetrahydropyranyl |
| IA-715 | P.395 | 3-tetrahydropyranyl | IA-716 | P.397 | 3-tetrahydropyranyl |
| IA-717 | P.402 | 3-tetrahydropyranyl | IA-718 | P.403 | 3-tetrahydropyranyl |
| IA-719 | P.404 | 3-tetrahydropyranyl | IA-720 | P.406 | 3-tetrahydropyranyl |
| IA-721 | H | 4-pyridyl | IA-722 | F | 4-pyridyl |
| IA-723 | Cl | 4-pyridyl | IA-724 | Br | 4-pyridyl |
| IA-725 | CH₃ | 4-pyridyl | IA-726 | CN | 4-pyridyl |
| IA-727 | CH═CH₂ | 4-pyridyl | IA-728 | CCH | 4-pyridyl |
| IA-729 | 2-pyridyl | 4-pyridyl | IA-730 | 3-pyridyl | 4-pyridyl |
| IA-731 | 4-pyridyl | 4-pyridyl | IA-732 | P.1 | 4-pyridyl |
| IA-733 | P.2 | 4-pyridyl | IA-734 | P.3 | 4-pyridyl |
| IA-735 | P.6 | 4-pyridyl | IA-736 | P.7 | 4-pyridyl |
| IA-737 | P.8 | 4-pyridy | IA-738 | P.20 | 4-pyridyl |
| IA-739 | P.48 | 4-pyridyl | IA-740 | P.49 | 4-pyridyl |
| IA-741 | P.52 | 4-pyridyl | IA-742 | P.53 | 4-pyridyl |
| IA-743 | P.54 | 4-pyridyl | IA-744 | P.66 | 4-pyridyl |
| IA-745 | P.101 | 4-pyridyl | IA-746 | P.102 | 4-pyridyl |
| IA-747 | P.111 | 4-pyridyl | IA-748 | P.112 | 4-pyridyl |
| IA-749 | P.127 | 4-pyridyl | IA-750 | P.128 | 4-pyridyl |
| IA-751 | P.273 | 4-pyridyl | IA-752 | P.274 | 4-pyridyl |
| IA-753 | P.393 | 4-pyridyl | IA-754 | P.394 | 4-pyridyl |
| IA-755 | P.395 | 4-pyridyl | IA-756 | P.397 | 4-pyridyl |
| IA-757 | P.402 | 4-pyridyl | IA-758 | P.403 | 4-pyridyl |
| IA-759 | P.404 | 4-pyridyl | IA-760 | P.406 | 4-pyridyl |
| IA-761 | H | 3-thiophenyl | IA-762 | F | 3-thiophenyl |
| IA-763 | Cl | 3-thiophenyl | IA-764 | Br | 3-thiophenyl |
| IA-765 | CH₃ | 3-thiophenyl | IA-766 | CN | 3-thiophenyl |
| IA-767 | CH═CH₂ | 3-thiophenyl | IA-768 | CCH | 3-thiophenyl |
| IA-769 | 2-pyridyl | 3-thiophenyl | IA-770 | 3-pyridyl | 3-thiophenyl |
| IA-771 | 4-pyridyl | 3-thiophenyl | IA-772 | P.1 | 3-thiophenyl |
| IA-773 | P.2 | 3-thiophenyl | IA-774 | P.3 | 3-thiophenyl |
| IA-775 | P.6 | 3-thiophenyl | IA-776 | P.7 | 3-thiophenyl |
| IA-777 | P.8 | 3-thiophenyl | IA-778 | P.20 | 3-thiophenyl |
| IA-779 | P.48 | 3-thiophenyl | IA-780 | P.49 | 3-thiophenyl |
| IA-781 | P.52 | 3-thiophenyl | IA-782 | P.53 | 3-thiophenyl |
| IA-783 | P.54 | 3-thiophenyl | IA-784 | P.66 | 3-thiophenyl |
| IA-785 | P.101 | 3-thiophenyl | IA-786 | P.102 | 3-thiophenyl |
| IA-787 | P.111 | 3-thiophenyl | IA-788 | P.112 | 3-thiophenyl |
| IA-789 | P.127 | 3-thiophenyl | IA-790 | P.128 | 3-thiophenyl |
| IA-791 | P.273 | 3-thiophenyl | IA-792 | P.274 | 3-thiophenyl |
| IA-793 | P.393 | 3-thiophenyl | IA-794 | P.394 | 3-thiophenyl |
| IA-795 | P.395 | 3-thiophenyl | IA-796 | P.397 | 3-thiophenyl |
| IA-797 | P.402 | 3-thiophenyl | IA-798 | P.403 | 3-thiophenyl |
| IA-799 | P.404 | 3-thiophenyl | IA-800 | P.406 | 3-thiophenyl |
| IA-801 | H | 3-furanyl | IA-802 | F | 3-furanyl |
| IA-803 | Cl | 3-furanyl | IA-804 | Br | 3-furanyl |
| IA-805 | CH₃ | 3-furanyl | IA-806 | CN | 3-furanyl |
| IA-807 | CH═CH₂ | 3-furanyl | IA-808 | CCH | 3-furanyl |
| IA-809 | 2-pyridyl | 3-furanyl | IA-810 | 3-pyridyl | 3-furanyl |
| IA-811 | 4-pyridyl | 3-furanyl | IA-812 | P.1 | 3-furanyl |
| IA-813 | P.2 | 3-furanyl | IA-814 | P.3 | 3-furanyl |
| IA-815 | P.6 | 3-furanyl | IA-816 | P.7 | 3-furanyl |
| IA-817 | P.8 | 3-furanyl | IA-818 | P.20 | 3-furanyl |
| IA-819 | P.48 | 3-furanyl | IA-820 | P.49 | 3-furanyl |
| IA-821 | P.52 | 3-furanyl | IA-822 | P.53 | 3-furanyl |
| IA-823 | P.54 | 3-furanyl | IA-824 | P.66 | 3-furanyl |
| IA-825 | P.101 | 3-furanyl | IA-826 | P.102 | 3-furanyl |
| IA-827 | P.111 | 3-furanyl | IA-828 | P.112 | 3-furanyl |
| IA-829 | P.127 | 3-furanyl | IA-830 | P.128 | 3-furanyl |
| IA-831 | P.273 | 3-furanyl | IA-832 | P.274 | 3-furanyl |
| IA-833 | P.393 | 3-furanyl | IA-834 | P.394 | 3-furanyl |
| IA-835 | P.395 | 3-furanyl | IA-836 | P.397 | 3-furanyl |
| IA-837 | P.402 | 3-furanyl | IA-838 | P.403 | 3-furanyl |
| IA-839 | P.404 | 3-furanyl | IA-840 | P.406 | 3-furanyl |
| IA-841 | H | 5-CH₃-isoxazol-3-yl | IA-842 | F | 5-CH₃-isoxazol-3-yl |
| IA-843 | Cl | 5-CH₃-isoxazol-3-yl | IA-844 | Br | 5-CH₃-isoxazol-3-yl |
| IA-845 | CH₃ | 5-CH₃-isoxazol-3-yl | IA-846 | CN | 5-CH₃-isoxazol-3-yl |
| IA-847 | CH═CH₂ | 5-CH₃-isoxazol-3-yl | IA-848 | CCH | 5-CH₃-isoxazol-3-yl |
| IA-849 | 2-pyridyl | 5-CH₃-isoxazol-3-yl | IA-850 | 3-pyridyl | 5-CH₃-isoxazol-3-yl |
| IA-851 | 4-pyridyl | 5-CH₃-isoxazol-3-yl | IA-852 | P.1 | 5-CH₃-isoxazol-3-yl |
| IA-853 | P.2 | 5-CH₃-isoxazol-3-yl | IA-854 | P.3 | 5-CH₃-isoxazol-3-yl |
| IA-855 | P.6 | 5-CH₃-isoxazol-3-yl | IA-856 | P.7 | 5-CH₃-isoxazol-3-yl |
| IA-857 | P.8 | 5-CH₃-isoxazol-3-yl | IA-858 | P.20 | 5-CH₃-isoxazol-3-yl |
| IA-859 | P.48 | 5-CH₃-isoxazol-3-yl | IA-860 | P.49 | 5-CH₃-isoxazol-3-yl |
| IA-861 | P.52 | 5-CH₃-isoxazol-3-yl | IA-862 | P.53 | 5-CH₃-isoxazol-3-yl |
| IA-863 | P.54 | 5-CH₃-isoxazol-3-yl | IA-864 | P.66 | 5-CH₃-isoxazol-3-yl |
| IA-865 | P.101 | 5-CH₃-isoxazol-3-yl | IA-866 | P.102 | 5-CH₃-isoxazol-3-yl |
| IA-867 | P.111 | 5-CH₃-isoxazol-3-yl | IA-868 | P.112 | 5-CH₃-isoxazol-3-yl |
| IA-869 | P.127 | 5-CH₃-isoxazol-3-yl | IA-870 | P.128 | 5-CH₃-isoxazol-3-yl |
| IA-871 | P.273 | 5-CH₃-isoxazol-3-yl | IA-872 | P.274 | 5-CH₃-isoxazol-3-yl |
| IA-873 | P.393 | 5-CH₃-isoxazol-3-yl | IA-874 | P.394 | 5-CH₃-isoxazol-3-yl |
| IA-875 | P.395 | 5-CH₃-isoxazol-3-yl | IA-876 | P.397 | 5-CH₃-isoxazol-3-yl |
| IA-877 | P.402 | 5-CH₃-isoxazol-3-yl | IA-878 | P.403 | 5-CH₃-isoxazol-3-yl |
| IA-879 | P.404 | 5-CH₃-isoxazol-3-yl | IA-880 | P.406 | 5-CH₃-isoxazol-3-yl |
| IA-881 | H | 3-CH₃-isoxazol-5-yl | IA-882 | F | 3-CH₃-isoxazol-5-yl |
| IA-883 | Cl | 3-CH₃-isoxazol-5-yl | IA-884 | Br | 3-CH₃-isoxazol-5-yl |
| IA-885 | CH₃ | 3-CH₃-isoxazol-5-yl | IA-886 | CN | 3-CH₃-isoxazol-5-yl |
| IA-887 | CH═CH₂ | 3-CH₃-isoxazol-5-yl | IA-888 | CCH | 3-CH₃-isoxazol-5-yl |
| IA-889 | 2-pyridyl | 3-CH₃-isoxazol-5-yl | IA-890 | 3-pyridyl | 3-CH₃-isoxazol-5-yl |
| IA-891 | 4-pyridyl | 3-CH₃-isoxazol-5-yl | IA-892 | P.1 | 3-CH₃-isoxazol-5-yl |
| IA-893 | P.2 | 3-CH₃-isoxazol-5-yl | IA-894 | P.3 | 3-CH₃-isoxazol-5-yl |
| IA-895 | P.6 | 3-CH₃-isoxazol-5-yl | IA-896 | P.7 | 3-CH₃-isoxazol-5-yl |
| IA-897 | P.8 | 3-CH₃-isoxazol-5-yl | IA-898 | P.20 | 3-CH₃-isoxazol-5-yl |
| IA-899 | P.48 | 3-CH₃-isoxazol-5-yl | IA-900 | P.49 | 3-CH₃-isoxazol-5-yl |
| IA-901 | P.52 | 3-CH₃-isoxazol-5-yl | IA-902 | P.53 | 3-CH₃-isoxazol-5-yl |
| IA-903 | P.54 | 3-CH₃-isoxazol-5-yl | IA-904 | P.66 | 3-CH₃-isoxazol-5-yl |
| IA-905 | P.101 | 3-CH₃-isoxazol-5-yl | IA-906 | P.102 | 3-CH₃-isoxazol-5-yl |
| IA-907 | P.111 | 3-CH₃-isoxazol-5-yl | IA-908 | P.112 | 3-CH₃-isoxazol-5-yl |
| IA-909 | P.127 | 3-CH₃-isoxazol-5-yl | IA-910 | P.128 | 3-CH₃-isoxazol-5-yl |
| IA-911 | P.273 | 3-CH₃-isoxazol-5-yl | IA-912 | P.274 | 3-CH₃-isoxazol-5-yl |
| IA-913 | P.393 | 3-CH₃-isoxazol-5-yl | IA-914 | P.394 | 3-CH₃-isoxazol-5-yl |
| IA-915 | P.395 | 3-CH₃-isoxazol-5-yl | IA-916 | P.397 | 3-CH₃-isoxazol-5-yl |
| IA-917 | P.402 | 3-CH₃-isoxazol-5-yl | IA-918 | P.403 | 3-CH₃-isoxazol-5-yl |
| IA-919 | P.404 | 3-CH₃-isoxazol-5-yl | IA-920 | P.406 | 3-CH₃-isoxazol-5-yl |
| IA-921 | H | P.1 | IA-922 | F | P.1 |
| IA-923 | Cl | P.1 | IA-924 | Br | P.1 |
| IA-925 | CH₃ | P.1 | IA-926 | CN | P.1 |
| IA-927 | CH═CH₂ | P.1 | IA-928 | CCH | P.1 |
| IA-929 | 2-pyridyl | P.1 | IA-930 | 3-pyridyl | P.1 |
| IA-931 | 4-pyridyl | P.1 | IA-932 | P.1 | P.1 |
| IA-933 | P.2 | P.1 | IA-934 | P.3 | P.1 |
| IA-935 | P.6 | P.1 | IA-936 | P.7 | P.1 |
| IA-937 | P.8 | P.1 | IA-938 | P.20 | P.1 |
| IA-939 | P.48 | P.1 | IA-940 | P.49 | P.1 |
| IA-941 | P.52 | P.1 | IA-942 | P.53 | P.1 |
| IA-943 | P.54 | P.1 | IA-944 | P.66 | P.1 |
| IA-945 | P.101 | P.1 | IA-946 | P.102 | P.1 |
| IA-947 | P.111 | P.1 | IA-948 | P.112 | P.1 |
| IA-949 | P.127 | P.1 | IA-950 | P.128 | P.1 |
| IA-951 | P.273 | P.1 | IA-952 | P.274 | P.1 |
| IA-953 | P.393 | P.1 | IA-954 | P.394 | P.1 |
| IA-955 | P.395 | P.1 | IA-956 | P.397 | P.1 |
| IA-957 | P.402 | P.1 | IA-958 | P.403 | P.1 |
| IA-959 | P.404 | P.1 | IA-960 | P.406 | P.1 |
| IA-961 | H | P.2 | IA-962 | F | P.2 |
| IA-963 | Cl | P.2 | IA-964 | Br | P.2 |
| IA-965 | CH₃ | P.2 | IA-966 | CN | P.2 |
| IA-967 | CH═CH₂ | P.2 | IA-968 | CCH | P.2 |
| IA-969 | 2-pyridyl | P.2 | IA-970 | 3-pyridyl | P.2 |
| IA-971 | 4-pyridyl | P.2 | IA-972 | P.1 | P.2 |
| IA-973 | P.2 | P.2 | IA-974 | P.3 | P.2 |
| IA-975 | P.6 | P.2 | IA-976 | P.7 | P.2 |
| IA-977 | P.8 | P.2 | IA-978 | P.20 | P.2 |
| IA-979 | P.48 | P.2 | IA-980 | P.49 | P.2 |
| IA-981 | P.52 | P.2 | IA-982 | P.53 | P.2 |
| IA-983 | P.54 | P.2 | IA-984 | P.66 | P.2 |
| IA-985 | P.101 | P.2 | IA-986 | P.102 | P.2 |
| IA-987 | P.111 | P.2 | IA-988 | P.112 | P.2 |
| IA-989 | P.127 | P.2 | IA-990 | P.128 | P.2 |
| IA-991 | P.273 | P.2 | IA-992 | P.274 | P.2 |
| IA-993 | P.393 | P.2 | IA-994 | P.394 | P.2 |
| IA-995 | P.395 | P.2 | IA-996 | P.397 | P.2 |
| IA-997 | P.402 | P.2 | IA-998 | P.403 | P.2 |
| IA-999 | P.404 | P.2 | IA-1000 | P.406 | P.2 |
| IA-1001 | H | P.3 | IA-1002 | F | P.3 |
| IA-1003 | Cl | P.3 | IA-1004 | Br | P.3 |
| IA-1005 | CH₃ | P.3 | IA-1006 | CN | P.3 |
| IA-1007 | CH═CH₂ | P.3 | IA-1008 | CCH | P.3 |
| IA-1009 | 2-pyridyl | P.3 | IA-1010 | 3-pyridyl | P.3 |
| IA-1011 | 4-pyridyl | P.3 | IA-1012 | P.1 | P.3 |
| IA-1013 | P.2 | P.3 | IA-1014 | P.3 | P.3 |
| IA-1015 | P.6 | P.3 | IA-1016 | P.7 | P.3 |
| IA-1017 | P.8 | P.3 | IA-1018 | P.20 | P.3 |
| IA-1019 | P.48 | P.3 | IA-1020 | P.49 | P.3 |
| IA-1021 | P.52 | P.3 | IA-1022 | P.53 | P.3 |
| IA-1023 | P.54 | P.3 | IA-1024 | P.66 | P.3 |
| IA-1025 | P.101 | P.3 | IA-1026 | P.102 | P.3 |
| IA-1027 | P.111 | P.3 | IA-1028 | P.112 | P.3 |
| IA-1029 | P.127 | P.3 | IA-1030 | P.128 | P.3 |
| IA-1031 | P.273 | P.3 | IA-1032 | P.274 | P.3 |
| IA-1033 | P.393 | P.3 | IA-1034 | P.394 | P.3 |
| IA-1035 | P.395 | P.3 | IA-1036 | P.397 | P.3 |
| IA-1037 | P.402 | P.3 | IA-1038 | P.403 | P.3 |
| IA-1039 | P.404 | P.3 | IA-1040 | P.406 | P.3 |
| IA-1041 | H | P.6 | IA-1042 | F | P.6 |
| IA-1043 | Cl | P.6 | IA-1044 | Br | P.6 |
| IA-1045 | CH₃ | P.6 | IA-1046 | CN | P.6 |
| IA-1047 | CH═CH₂ | P.6 | IA-1048 | CCH | P.6 |
| IA-1049 | 2-pyridyl | P.6 | IA-1050 | 3-pyridyl | P.6 |
| IA-1051 | 4-pyridyl | P.6 | IA-1052 | P.1 | P.6 |
| IA-1053 | P.2 | P.6 | IA-1054 | P.3 | P.6 |
| IA-1055 | P.6 | P.6 | IA-1056 | P.7 | P.6 |
| IA-1057 | P.8 | P.6 | IA-1058 | P.20 | P.6 |
| IA-1059 | P.48 | P.6 | IA-1060 | P.49 | P.6 |
| IA-1061 | P.52 | P.6 | IA-1062 | P.53 | P.6 |
| IA-1063 | P.54 | P.6 | IA-1064 | P.66 | P.6 |
| IA-1065 | P.101 | P.6 | IA-1066 | P.102 | P.6 |
| IA-1067 | P.111 | P.6 | IA-1068 | P.112 | P.6 |
| IA-1069 | P.127 | P.6 | IA-1070 | P.128 | P.6 |
| IA-1071 | P.273 | P.6 | IA-1072 | P.274 | P.6 |
| IA-1073 | P.393 | P.6 | IA-1074 | P.394 | P.6 |
| IA-1075 | P.395 | P.6 | IA-1076 | P.397 | P.6 |
| IA-1077 | P.402 | P.6 | IA-1078 | P.403 | P.6 |
| IA-1079 | P.404 | P.6 | IA-1080 | P.406 | P.6 |
| IA-1081 | H | P.7 | IA-1082 | F | P.7 |
| IA-1083 | Cl | P.7 | IA-1084 | Br | P.7 |
| IA-1085 | CH₃ | P.7 | IA-1086 | CN | P.7 |
| IA-1087 | CH═CH₂ | P.7 | IA-1088 | CCH | P.7 |
| IA-1089 | 2-pyridyl | P.7 | IA-1090 | 3-pyridyl | P.7 |
| IA-1091 | 4-pyridyl | P.7 | IA-1092 | P.1 | P.7 |
| IA-1093 | P.2 | P.7 | IA-1094 | P.3 | P.7 |
| IA-1095 | P.6 | P.7 | IA-1096 | P.7 | P.7 |
| IA-1097 | P.8 | P.7 | IA-1098 | P.20 | P.7 |
| IA-1099 | P.48 | P.7 | IA-1100 | P.49 | P.7 |
| IA-1101 | P.52 | P.7 | IA-1102 | P.53 | P.7 |
| IA-1103 | P.54 | P.7 | IA-1104 | P.66 | P.7 |
| IA-1105 | P.101 | P.7 | IA-1106 | P.102 | P.7 |
| IA-1107 | P.111 | P.7 | IA-1108 | P.112 | P.7 |
| IA-1109 | P.127 | P.7 | IA-1110 | P.128 | P.7 |
| IA-1111 | P.273 | P.7 | IA-1112 | P.274 | P.7 |
| IA-1113 | P.393 | P.7 | IA-1114 | P.394 | P.7 |
| IA-1115 | P.395 | P.7 | IA-1116 | P.397 | P.7 |
| IA-1117 | P.402 | P.7 | IA-1118 | P.403 | P.7 |
| IA-1119 | P.404 | P.7 | IA-1120 | P.406 | P.7 |
| IA-1121 | H | P.8 | IA-1122 | F | P.8 |
| IA-1123 | Cl | P.8 | IA-1124 | Br | P.8 |
| IA-1125 | CH₃ | P.8 | IA-1126 | CN | P.8 |
| IA-1127 | CH═CH₂ | P.8 | IA-1128 | CCH | P.8 |
| IA-1129 | 2-pyridyl | P.8 | IA-1130 | 3-pyridyl | P.8 |
| IA-1131 | 4-pyridyl | P.8 | IA-1132 | P.1 | P.8 |
| IA-1133 | P.2 | P.8 | IA-1134 | P.3 | P.8 |
| IA-1135 | P.6 | P.8 | IA-1136 | P.7 | P.8 |
| IA-1137 | P.8 | P.8 | IA-1138 | P.20 | P.8 |
| IA-1139 | P.48 | P.8 | IA-1140 | P.49 | P.8 |
| IA-1141 | P.52 | P.8 | IA-1142 | P.53 | P.8 |
| IA-1143 | P.54 | P.8 | IA-1144 | P.66 | P.8 |
| IA-1145 | P.101 | P.8 | IA-1146 | P.102 | P.8 |
| IA-1147 | P.111 | P.8 | IA-1148 | P.112 | P.8 |
| IA-1149 | P.127 | P.8 | IA-1150 | P.128 | P.8 |
| IA-1151 | P.273 | P.8 | IA-1152 | P.274 | P.8 |
| IA-1153 | P.393 | P.8 | IA-1154 | P.394 | P.8 |
| IA-1155 | P.395 | P.8 | IA-1156 | P.397 | P.8 |
| IA-1157 | P.402 | P.8 | IA-1158 | P.403 | P.8 |
| IA-1159 | P.404 | P.8 | IA-1160 | P.406 | P.8 |
| IA-1161 | H | P.12 | IA-1162 | F | P.12 |
| IA-1163 | Cl | P.12 | IA-1164 | Br | P.12 |
| IA-1165 | CH₃ | P.12 | IA-1166 | CN | P.12 |
| IA-1167 | CH═CH₂ | P.12 | IA-1168 | CCH | P.12 |
| IA-1169 | 2-pyridyl | P.12 | IA-1170 | 3-pyridyl | P.12 |
| IA-1171 | 4-pyridyl | P.12 | IA-1172 | P.1 | P.12 |
| IA-1173 | P.2 | P.12 | IA-1174 | P.3 | P.12 |
| IA-1175 | P.6 | P.12 | IA-1176 | P.7 | P.12 |
| IA-1177 | P.8 | P.12 | IA-1178 | P.20 | P.12 |
| IA-1179 | P.48 | P.12 | IA-1180 | P.49 | P.12 |
| IA-1181 | P.52 | P.12 | IA-1182 | P.53 | P.12 |
| IA-1183 | P.54 | P.12 | IA-1184 | P.66 | P.12 |
| IA-1185 | P.101 | P.12 | IA-1186 | P.102 | P.12 |
| IA-1187 | P.111 | P.12 | IA-1188 | P.112 | P.12 |
| IA-1189 | P.127 | P.12 | IA-1190 | P.128 | P.12 |
| IA-1191 | P.273 | P.12 | IA-1192 | P.274 | P.12 |
| IA-1193 | P.393 | P.12 | IA-1194 | P.394 | P.12 |
| IA-1195 | P.395 | P.12 | IA-1196 | P.397 | P.12 |
| IA-1197 | P.402 | P.12 | IA-1198 | P.403 | P.12 |
| IA-1199 | P.404 | P.12 | IA-1200 | P.406 | P.12 |
| IA-1201 | H | P.14 | IA-1202 | F | P.14 |
| IA-1203 | Cl | P.14 | IA-1204 | Br | P.14 |
| IA-1205 | CH₃ | P.14 | IA-1206 | CN | P.14 |
| IA-1207 | CH═CH₂ | P.14 | IA-1208 | CCH | P.14 |
| IA-1209 | 2-pyridyl | P.14 | IA-1210 | 3-pyridyl | P.14 |
| IA-1211 | 4-pyridyl | P.14 | IA-1212 | P.1 | P.14 |
| IA-1213 | P.2 | P.14 | IA-1214 | P.3 | P.14 |
| IA-1215 | P.6 | P.14 | IA-1216 | P.7 | P.14 |
| IA-1217 | P.8 | P.14 | IA-1218 | P.20 | P.14 |
| IA-1219 | P.48 | P.14 | IA-1220 | P.49 | P.14 |
| IA-1221 | P.52 | P.14 | IA-1222 | P.53 | P.14 |
| IA-1223 | P.54 | P.14 | IA-1224 | P.66 | P.14 |
| IA-1225 | P.101 | P.14 | IA-1226 | P.102 | P.14 |
| IA-1227 | P.111 | P.14 | IA-1228 | P.112 | P.14 |
| IA-1229 | P.127 | P.14 | IA-1230 | P.128 | P.14 |
| IA-1231 | P.273 | P.14 | IA-1232 | P.274 | P.14 |
| IA-1233 | P.393 | P.14 | IA-1234 | P.394 | P.14 |
| IA-1235 | P.395 | P.14 | IA-1236 | P.397 | P.14 |
| IA-1237 | P.402 | P.14 | IA-1238 | P.403 | P.14 |
| IA-1239 | P.404 | P.14 | IA-1240 | P.406 | P.14 |
| IA-1241 | H | P.19 | IA-1242 | F | P.19 |
| IA-1243 | Cl | P.19 | IA-1244 | Br | P.19 |
| IA-1245 | CH₃ | P.19 | IA-1246 | CN | P.19 |
| IA-1247 | CH═CH₂ | P.19 | IA-1248 | CCH | P.19 |
| IA-1249 | 2-pyridyl | P.19 | IA-1250 | 3-pyridyl | P.19 |
| IA-1251 | 4-pyridyl | P.19 | IA-1252 | P.1 | P.19 |
| IA-1253 | P.2 | P.19 | IA-1254 | P.3 | P.19 |
| IA-1255 | P.6 | P.19 | IA-1256 | P.7 | P.19 |
| IA-1257 | P.8 | P.19 | IA-1258 | P.20 | P.19 |
| IA-1259 | P.48 | P.19 | IA-1260 | P.49 | P.19 |
| IA-1261 | P.52 | P.19 | IA-1262 | P.53 | P.19 |
| IA-1263 | P.54 | P.19 | IA-1264 | P.66 | P.19 |
| IA-1265 | P.101 | P.19 | IA-1266 | P.102 | P.19 |
| IA-1267 | P.111 | P.19 | IA-1268 | P.112 | P.19 |
| IA-1269 | P.127 | P.19 | IA-1270 | P.128 | P.19 |
| IA-1271 | P.273 | P.19 | IA-1272 | P.274 | P.19 |
| IA-1273 | P.393 | P.19 | IA-1274 | P.394 | P.19 |
| IA-1275 | P.395 | P.19 | IA-1276 | P.397 | P.19 |
| IA-1277 | P.402 | P.19 | IA-1278 | P.403 | P.19 |
| IA-1279 | P.404 | P.19 | IA-1280 | P.406 | P.19 |
| IA-1281 | H | P.20 | IA-1282 | F | P.20 |
| IA-1283 | Cl | P.20 | IA-1284 | Br | P.20 |
| IA-1285 | CH₃ | P.20 | IA-1286 | CN | P.20 |
| IA-1287 | CH═CH₂ | P.20 | IA-1288 | CCH | P.20 |
| IA-1289 | 2-pyridyl | P.20 | IA-1290 | 3-pyridyl | P.20 |
| IA-1291 | 4-pyridyl | P.20 | IA-1292 | P.1 | P.20 |
| IA-1293 | P.2 | P.20 | IA-1294 | P.3 | P.20 |
| IA-1295 | P.6 | P.20 | IA-1296 | P.7 | P.20 |
| IA-1297 | P.8 | P.20 | IA-1298 | P.20 | P.20 |
| IA-1299 | P.48 | P.20 | IA-1300 | P.49 | P.20 |
| IA-1301 | P.52 | P.20 | IA-1302 | P.53 | P.20 |
| IA-1303 | P.54 | P.20 | IA-1304 | P.66 | P.20 |
| IA-1305 | P.101 | P.20 | IA-1306 | P.102 | P.20 |
| IA-1307 | P.111 | P.20 | IA-1308 | P.112 | P.20 |
| IA-1309 | P.127 | P.20 | IA-1310 | P.128 | P.20 |
| IA-1311 | P.273 | P.20 | IA-1312 | P.274 | P.20 |
| IA-1313 | P.393 | P.20 | IA-1314 | P.394 | P.20 |
| IA-1315 | P.395 | P.20 | IA-1316 | P.397 | P.20 |
| IA-1317 | P.402 | P.20 | IA-1318 | P.403 | P.20 |
| IA-1319 | P.404 | P.20 | IA-1320 | P.406 | P.20 |
| IA-1321 | H | P.23 | IA-1322 | F | P.23 |
| IA-1323 | Cl | P.23 | IA-1324 | Br | P.23 |
| IA-1325 | CH₃ | P.23 | IA-1326 | CN | P.23 |
| IA-1327 | CH═CH₂ | P.23 | IA-1328 | CCH | P.23 |
| IA-1329 | 2-pyridyl | P.23 | IA-1330 | 3-pyridyl | P.23 |
| IA-1331 | 4-pyridyl | P.23 | IA-1332 | P.1 | P.23 |
| IA-1333 | P.2 | P.23 | IA-1334 | P.3 | P.23 |
| IA-1335 | P.6 | P.23 | IA-1336 | P.7 | P.23 |
| IA-1337 | P.8 | P.23 | IA-1338 | P.20 | P.23 |
| IA-1339 | P.48 | P.23 | IA-1340 | P.49 | P.23 |
| IA-1341 | P.52 | P.23 | IA-1342 | P.53 | P.23 |
| IA-1343 | P.54 | P.23 | IA-1344 | P.66 | P.23 |
| IA-1345 | P.101 | P.23 | IA-1346 | P.102 | P.23 |
| IA-1347 | P.111 | P.23 | IA-1348 | P.112 | P.23 |
| IA-1349 | P.127 | P.23 | IA-1350 | P.128 | P.23 |
| IA-1351 | P.273 | P.23 | IA-1352 | P.274 | P.23 |
| IA-1353 | P.393 | P.23 | IA-1354 | P.394 | P.23 |
| IA-1355 | P.395 | P.23 | IA-1356 | P.397 | P.23 |
| IA-1357 | P.402 | P.23 | IA-1358 | P.403 | P.23 |
| IA-1359 | P.404 | P.23 | IA-1360 | P.406 | P.23 |
| IA-1361 | H | P.25 | IA-1362 | F | P.25 |
| IA-1363 | Cl | P.25 | IA-1364 | Br | P.25 |
| IA-1365 | CH₃ | P.25 | IA-1366 | CN | P.25 |
| IA-1367 | CH═CH₂ | P.25 | IA-1368 | CCH | P.25 |
| IA-1369 | 2-pyridyl | P.25 | IA-1370 | 3-pyridyl | P.25 |
| IA-1371 | 4-pyridyl | P.25 | IA-1372 | P.1 | P.25 |
| IA-1373 | P.2 | P.25 | IA-1374 | P.3 | P.25 |
| IA-1375 | P.6 | P.25 | IA-1376 | P.7 | P.25 |
| IA-1377 | P.8 | P.25 | IA-1378 | P.20 | P.25 |
| IA-1379 | P.48 | P.25 | IA-1380 | P.49 | P.25 |
| IA-1381 | P.52 | P.25 | IA-1382 | P.53 | P.25 |
| IA-1383 | P.54 | P.25 | IA-1384 | P.66 | P.25 |
| IA-1385 | P.101 | P.25 | IA-1386 | P.102 | P.25 |
| IA-1387 | P.111 | P.25 | IA-1388 | P.112 | P.25 |
| IA-1389 | P.127 | P.25 | IA-1390 | P.128 | P.25 |
| IA-1391 | P.273 | P.25 | IA-1392 | P.274 | P.25 |
| IA-1393 | P.393 | P.25 | IA-1394 | P.394 | P.25 |
| IA-1395 | P.395 | P.25 | IA-1396 | P.397 | P.25 |
| IA-1397 | P.402 | P.25 | IA-1398 | P.403 | P.25 |
| IA-1399 | P.404 | P.25 | IA-1400 | P.406 | P.25 |
| IA-1401 | H | P.28 | IA-1402 | F | P.28 |
| IA-1403 | Cl | P.28 | IA-1404 | Br | P.28 |
| IA-1405 | CH₃ | P.28 | IA-1406 | CN | P.28 |
| IA-1407 | CH═CH₂ | P.28 | IA-1408 | CCH | P.28 |
| IA-1409 | 2-pyridyl | P.28 | IA-1410 | 3-pyridyl | P.28 |
| IA-1411 | 4-pyridyl | P.28 | IA-1412 | P.1 | P.28 |
| IA-1413 | P.2 | P.28 | IA-1414 | P.3 | P.28 |
| IA-1415 | P.6 | P.28 | IA-1416 | P.7 | P.28 |
| IA-1417 | P.8 | P.28 | IA-1418 | P.20 | P.28 |
| IA-1419 | P.48 | P.28 | IA-1420 | P.49 | P.28 |
| IA-1421 | P.52 | P.28 | IA-1422 | P.53 | P.28 |
| IA-1423 | P.54 | P.28 | IA-1424 | P.66 | P.28 |
| IA-1425 | P.101 | P.28 | IA-1426 | P.102 | P.28 |
| IA-1427 | P.111 | P.28 | IA-1428 | P.112 | P.28 |
| IA-1429 | P.127 | P.28 | IA-1430 | P.128 | P.28 |
| IA-1431 | P.273 | P.28 | IA-1432 | P.274 | P.28 |
| IA-1433 | P.393 | P.28 | IA-1434 | P.394 | P.28 |
| IA-1435 | P.395 | P.28 | IA-1436 | P.397 | P.28 |
| IA-1437 | P.402 | P.28 | IA-1438 | P.403 | P.28 |
| IA-1439 | P.404 | P.28 | IA-1440 | P.406 | P.28 |
| IA-1441 | H | P.33 | IA-1442 | F | P.33 |
| IA-1443 | Cl | P.33 | IA-1444 | Br | P.33 |
| IA-1445 | CH₃ | P.33 | IA-1446 | CN | P.33 |
| IA-1447 | CH═CH₂ | P.33 | IA-1448 | CCH | P.33 |
| IA-1449 | 2-pyridyl | P.33 | IA-1450 | 3-pyridyl | P.33 |
| IA-1451 | 4-pyridyl | P.33 | IA-1452 | P.1 | P.33 |
| IA-1453 | P.2 | P.33 | IA-1454 | P.3 | P.33 |
| IA-1455 | P.6 | P.33 | IA-1456 | P.7 | P.33 |
| IA-1457 | P.8 | P.33 | IA-1458 | P.20 | P.33 |
| IA-1459 | P.48 | P.33 | IA-1460 | P.49 | P.33 |
| IA-1461 | P.52 | P.33 | IA-1462 | P.53 | P.33 |
| IA-1463 | P.54 | P.33 | IA-1464 | P.66 | P.33 |
| IA-1465 | P.101 | P.33 | IA-1466 | P.102 | P.33 |
| IA-1467 | P.111 | P.33 | IA-1468 | P.112 | P.33 |
| IA-1469 | P.127 | P.33 | IA-1470 | P.128 | P.33 |
| IA-1471 | P.273 | P.33 | IA-1472 | P.274 | P.33 |
| IA-1473 | P.393 | P.33 | IA-1474 | P.394 | P.33 |
| IA-1475 | P.395 | P.33 | IA-1476 | P.397 | P.33 |
| IA-1477 | P.402 | P.33 | IA-1478 | P.403 | P.33 |
| IA-1479 | P.404 | P.33 | IA-1480 | P.406 | P.33 |
| IA-1481 | H | P.42 | IA-1482 | F | P.42 |
| IA-1483 | Cl | P.42 | IA-1484 | Br | P.42 |
| IA-1485 | CH₃ | P.42 | IA-1486 | CN | P.42 |
| IA-1487 | CH═CH₂ | P.42 | IA-1488 | CCH | P.42 |
| IA-1489 | 2-pyridyl | P.42 | IA-1490 | 3-pyridyl | P.42 |
| IA-1491 | 4-pyridyl | P.42 | IA-1492 | P.1 | P.42 |
| IA-1493 | P.2 | P.42 | IA-1494 | P.3 | P.42 |
| IA-1495 | P.6 | P.42 | IA-1496 | P.7 | P.42 |
| IA-1497 | P.8 | P.42 | IA-1498 | P.20 | P.42 |
| IA-1499 | P.48 | P.42 | IA-1500 | P.49 | P.42 |
| IA-1501 | P.52 | P.42 | IA-1502 | P.53 | P.42 |
| IA-1503 | P.54 | P.42 | IA-1504 | P.66 | P.42 |
| IA-1505 | P.101 | P.42 | IA-1506 | P.102 | P.42 |
| IA-1507 | P.111 | P.42 | IA-1508 | P.112 | P.42 |
| IA-1509 | P.127 | P.42 | IA-1510 | P.128 | P.42 |
| IA-1511 | P.273 | P.42 | IA-1512 | P.274 | P.42 |
| IA-1513 | P.393 | P.42 | IA-1514 | P.394 | P.42 |
| IA-1515 | P.395 | P.42 | IA-1516 | P.397 | P.42 |
| IA-1517 | P.402 | P.42 | IA-1518 | P.403 | P.42 |
| IA-1519 | P.404 | P.42 | IA-1520 | P.406 | P.42 |
| IA-1521 | H | P.45 | IA-1522 | F | P.45 |
| IA-1523 | Cl | P.45 | IA-1524 | Br | P.45 |
| IA-1525 | CH₃ | P.45 | IA-1526 | CN | P.45 |
| IA-1527 | CH═CH₂ | P.45 | IA-1528 | CCH | P.45 |
| IA-1529 | 2-pyridyl | P.45 | IA-1530 | 3-pyridyl | P.45 |
| IA-1531 | 4-pyridyl | P.45 | IA-1532 | P.1 | P.45 |
| IA-1533 | P.2 | P.45 | IA-1534 | P.3 | P.45 |
| IA-1535 | P.6 | P.45 | IA-1536 | P.7 | P.45 |
| IA-1537 | P.8 | P.45 | IA-1538 | P.20 | P.45 |
| IA-1539 | P.48 | P.45 | IA-1540 | P.49 | P.45 |
| IA-1541 | P.52 | P.45 | IA-1542 | P.53 | P.45 |
| IA-1543 | R.54 | P.45 | IA-1544 | P.66 | P.45 |
| IA-1545 | P.101 | P.45 | IA-1546 | P.102 | P.45 |
| IA-1547 | P.111 | P.45 | IA-1548 | P.112 | P.45 |
| IA-1549 | P.127 | P.45 | IA-1550 | P.128 | P.45 |
| IA-1551 | P.273 | P.45 | IA-1552 | P.274 | P.45 |
| IA-1553 | P.393 | P.45 | IA-1554 | P.394 | P.45 |
| IA-1555 | P.395 | P.45 | IA-1556 | P.397 | P.45 |
| IA-1557 | P.402 | P.45 | IA-1558 | P.403 | P.45 |
| IA-1559 | P.404 | P.45 | IA-1560 | P.406 | P.45 |
| IA-1561 | H | P.48 | IA-1562 | F | P.48 |
| IA-1563 | Cl | P.48 | IA-1564 | Br | P.48 |
| IA-1565 | CH₃ | P.48 | IA-1566 | CN | P.48 |
| IA-1567 | CH═CH₂ | P.48 | IA-1568 | CCH | P.48 |
| IA-1569 | 2-pyridyl | P.48 | IA-1570 | 3-pyridyl | P.48 |
| IA-1571 | 4-pyridyl | P.48 | IA-1572 | P.1 | P.48 |
| IA-1573 | P.2 | P.48 | IA-1574 | P.3 | P.48 |
| IA-1575 | P.6 | P.48 | IA-1576 | P.7 | P.48 |
| IA-1577 | P.8 | P.48 | IA-1578 | P.20 | P.48 |
| IA-1579 | P.48 | P.48 | IA-1580 | P.49 | P.48 |
| IA-1581 | P.52 | P.48 | IA-1582 | P.53 | P.48 |
| IA-1583 | P.54 | P.48 | IA-1584 | P.66 | P.48 |
| IA-1585 | P.101 | P.48 | IA-1586 | P.102 | P.48 |
| IA-1587 | P.111 | P.48 | IA-1588 | P.112 | P.48 |
| IA-1589 | P.127 | P.48 | IA-1590 | P.128 | P.48 |
| IA-1591 | P.273 | P.48 | IA-1592 | P.274 | P.48 |
| IA-1593 | P.393 | P.48 | IA-1594 | P.394 | P.48 |
| IA-1595 | P.395 | P.48 | IA-1596 | P.397 | P.48 |
| IA-1597 | P.402 | P.48 | IA-1598 | P.403 | P.48 |
| IA-1599 | P.404 | P.48 | IA-1600 | P.406 | P.48 |
| IA-1601 | H | P.49 | IA-1602 | F | P.49 |
| IA-1603 | Cl | P.49 | IA-1604 | Br | P.49 |
| IA-1605 | CH₃ | P.49 | IA-1606 | CN | P.49 |
| IA-1607 | CH═CH₂ | P.49 | IA-1608 | CCH | P.49 |
| IA-1609 | 2-pyridyl | P.49 | IA-1610 | 3-pyridyl | P.49 |
| IA-1611 | 4-pyridyl | P.49 | IA-1612 | P.1 | P.49 |
| IA-1613 | P.2 | P.49 | IA-1614 | P.3 | P.49 |
| IA-1615 | P.6 | P.49 | IA-1616 | P.7 | P.49 |
| IA-1617 | P.8 | P.49 | IA-1618 | P.20 | P.49 |
| IA-1619 | P.48 | P.49 | IA-1620 | P.49 | P.49 |
| IA-1621 | P.52 | P.49 | IA-1622 | P.53 | P.49 |
| IA-1623 | P.54 | P.49 | IA-1624 | P.66 | P.49 |
| IA-1625 | P.101 | P.49 | IA-1626 | P.102 | P.49 |
| IA-1627 | P.111 | P.49 | IA-1628 | P.112 | P.49 |
| IA-1629 | P.127 | P.49 | IA-1630 | P.128 | P.49 |
| IA-1631 | P.273 | P.49 | IA-1632 | P.274 | P.49 |
| IA-1633 | P.393 | P.49 | IA-1634 | P.394 | P.49 |
| IA-1635 | P.395 | P.49 | IA-1636 | P.397 | P.49 |
| IA-1637 | P.402 | P.49 | IA-1638 | P.403 | P.49 |
| IA-1639 | P.404 | P.49 | IA-1640 | P.406 | P.49 |
| IA-1641 | H | P.52 | IA-1642 | F | P.52 |
| IA-1643 | Cl | P.52 | IA-1644 | Br | P.52 |
| IA-1645 | CH₃ | P.52 | IA-1646 | CN | P.52 |
| IA-1647 | CH═CH₂ | P.52 | IA-1648 | CCH | P.52 |
| IA-1649 | 2-pyridyl | P.52 | IA-1650 | 3-pyridyl | P.52 |
| IA-1651 | 4-pyridyl | P.52 | IA-1652 | P.1 | P.52 |
| IA-1653 | P.2 | P.52 | IA-1654 | P.3 | P.52 |
| IA-1655 | P.6 | P.52 | IA-1656 | P.7 | P.52 |
| IA-1657 | P.8 | P.52 | IA-1658 | P.20 | P.52 |
| IA-1659 | P.48 | P.52 | IA-1660 | P.49 | P.52 |
| IA-1661 | P.52 | P.52 | IA-1662 | P.53 | P.52 |
| IA-1663 | P.54 | P.52 | IA-1664 | P.66 | P.52 |
| IA-1665 | P.101 | P.52 | IA-1666 | P.102 | P.52 |
| IA-1667 | P.111 | P.52 | IA-1668 | P.112 | P.52 |
| IA-1669 | P.127 | P.52 | IA-1670 | P.128 | P.52 |
| IA-1671 | P.273 | P.52 | IA-1672 | P.274 | P.52 |
| IA-1673 | P.393 | P.52 | IA-1674 | P.394 | P.52 |
| IA-1675 | P.395 | P.52 | IA-1676 | P.397 | P.52 |
| IA-1677 | P.402 | P.52 | IA-1678 | P.403 | P.52 |
| IA-1679 | P.404 | P.52 | IA-1680 | P.406 | P.52 |
| IA-1681 | H | P.53 | IA-1682 | F | P.53 |
| IA-1683 | Cl | P.53 | IA-1684 | Br | P.53 |
| IA-1685 | CH₃ | P.53 | IA-1686 | CN | P.53 |
| IA-1687 | CH═CH₂ | P.53 | IA-1688 | CCH | P.53 |
| IA-1689 | 2-pyridyl | P.53 | IA-1690 | 3-pyridyl | P.53 |
| IA-1691 | 4-pyridyl | P.53 | IA-1692 | P.1 | P.53 |
| IA-1693 | P.2 | P.53 | IA-1694 | P.3 | P.53 |
| IA-1695 | P.6 | P.53 | IA-1696 | P.7 | P.53 |
| IA-1697 | P.8 | P.53 | IA-1698 | P.20 | P.53 |
| IA-1699 | P.48 | P.53 | IA-1700 | P.49 | P.53 |
| IA-1701 | P.52 | P.53 | IA-1702 | P.53 | P.53 |
| IA-1703 | P.54 | P.53 | IA-1704 | P.66 | P.53 |
| IA-1705 | P.101 | P.53 | IA-1706 | P.102 | P.53 |
| IA-1707 | P.111 | P.53 | IA-1708 | P.112 | P.53 |
| IA-1709 | P.127 | P.53 | IA-1710 | P.128 | P.53 |
| IA-1711 | P.273 | P.53 | IA-1712 | P.274 | P.53 |
| IA-1713 | P.393 | P.53 | IA-1714 | P.394 | P.53 |
| IA-1715 | P.395 | P.53 | IA-1716 | P.397 | P.53 |
| IA-1717 | P.402 | P.53 | IA-1718 | P.403 | P.53 |
| IA-1719 | P.404 | P.53 | IA-1720 | P.406 | P.53 |
| IA-1721 | H | P.54 | IA-1722 | F | P.54 |
| IA-1723 | Cl | P.54 | IA-1724 | Br | P.54 |
| IA-1725 | CH₃ | P.54 | IA-1726 | CN | P.54 |
| IA-1727 | CH═CH₂ | P.54 | IA-1728 | CCH | P.54 |
| IA-1729 | 2-pyridyl | P.54 | IA-1730 | 3-pyridyl | P.54 |
| IA-1731 | 4-pyridyl | P.54 | IA-1732 | P.1 | P.54 |
| IA-1733 | P.2 | P.54 | IA-1734 | P.3 | P.54 |
| IA-1735 | P.6 | P.54 | IA-1736 | P.7 | P.54 |
| IA-1737 | P.8 | P.54 | IA-1738 | P.20 | P.54 |
| IA-1739 | P.48 | P.54 | IA-1740 | P.49 | P.54 |
| IA-1741 | R.52 | P.54 | IA-1742 | P.53 | P.54 |
| IA-1743 | P.54 | P.54 | IA-1744 | P.66 | P.54 |
| IA-1745 | P.101 | P.54 | IA-1746 | P.102 | P.54 |
| IA-1747 | P.111 | P.54 | IA-1748 | P.112 | P.54 |
| IA-1749 | P.127 | P.54 | IA-1750 | P.128 | P.54 |
| IA-1751 | P.273 | P.54 | IA-1752 | P.274 | P.54 |
| IA-1753 | P.393 | P.54 | IA-1754 | P.394 | P.54 |
| IA-1755 | P.395 | P.54 | IA-1756 | P.397 | P.54 |
| IA-1757 | P.402 | P.54 | IA-1758 | P.403 | P.54 |
| IA-1759 | P.404 | P.54 | IA-1760 | P.406 | P.54 |
| IA-1761 | H | P.65 | IA-1762 | F | P.65 |
| IA-1763 | Cl | P.65 | IA-1764 | Br | P.65 |
| IA-1765 | CH₃ | P.65 | IA-1766 | CN | P.65 |
| IA-1767 | CH═CH₂ | P.65 | IA-1768 | CCH | P.65 |
| IA-1769 | 2-pyridyl | P.65 | IA-1770 | 3-pyridyl | P.65 |
| IA-1771 | 4-pyridyl | P.65 | IA-1772 | P.1 | P.65 |
| IA-1773 | P.2 | P.65 | IA-1774 | P.3 | P.65 |
| IA-1775 | P.6 | P.65 | IA-1776 | P.7 | P.65 |
| IA-1777 | P.8 | P.65 | IA-1778 | P.20 | P.65 |
| IA-1779 | P.48 | P.65 | IA-1780 | P.49 | P.65 |
| IA-1781 | P.52 | P.65 | IA-1782 | P.53 | P.65 |
| IA-1783 | P.54 | P.65 | IA-1784 | P.66 | P.65 |
| IA-1785 | P.101 | P.65 | IA-1786 | P.102 | P.65 |
| IA-1787 | P.111 | P.65 | IA-1788 | P.112 | P.65 |
| IA-1789 | P.127 | P.65 | IA-1790 | P.128 | P.65 |
| IA-1791 | P.273 | P.65 | IA-1792 | P.274 | P.65 |
| IA-1793 | P.393 | P.65 | IA-1794 | P.394 | P.65 |
| IA-1795 | P.395 | P.65 | IA-1796 | P.397 | P.65 |
| IA-1797 | P.402 | P.65 | IA-1798 | P.403 | P.65 |
| IA-1799 | P.404 | P.65 | IA-1800 | P.406 | P.65 |
| IA-1801 | H | P.66 | IA-1802 | F | P.66 |
| IA-1803 | Cl | P.66 | IA-1804 | Br | P.66 |
| IA-1805 | CH₃ | P.66 | IA-1806 | CN | P.66 |
| IA-1807 | CH═CH₂ | P.66 | IA-1808 | CCH | P.66 |
| IA-1809 | 2-pyridyl | P.66 | IA-1810 | 3-pyridyl | P.66 |
| IA-1811 | 4-pyridyl | P.66 | IA-1812 | P.1 | P.66 |
| IA-1813 | P.2 | P.66 | IA-1814 | P.3 | P.66 |
| IA-1815 | P.6 | P.66 | IA-1816 | P.7 | P.66 |
| IA-1817 | P.8 | P.66 | IA-1818 | P.20 | P.66 |
| IA-1819 | P.48 | P.66 | IA-1820 | P.49 | P.66 |
| IA-1821 | P.52 | P.66 | IA-1822 | P.53 | P.66 |
| IA-1823 | P.54 | P.66 | IA-1824 | P.66 | P.66 |
| IA-1825 | P.101 | P.66 | IA-1826 | P.102 | P.66 |
| IA-1827 | P.111 | P.66 | IA-1828 | P.112 | P.66 |
| IA-1829 | P.127 | P.66 | IA-1830 | P.128 | P.66 |
| IA-1831 | P.273 | P.66 | IA-1832 | P.274 | P.66 |
| IA-1833 | P.393 | P.66 | IA-1834 | P.394 | P.66 |
| IA-1835 | P.395 | P.66 | IA-1836 | P.397 | P.66 |
| IA-1837 | P.402 | P.66 | IA-1838 | P.403 | P.66 |
| IA-1839 | P.404 | P.66 | IA-1840 | P.406 | P.66 |
| IA-1841 | H | P.71 | IA-1842 | F | P.71 |
| IA-1843 | Cl | P.71 | IA-1844 | Br | P.71 |
| IA-1845 | CH₃ | P.71 | IA-1846 | CN | P.71 |
| IA-1847 | CH═CH₂ | P.71 | IA-1848 | CCH | P.71 |
| IA-1849 | 2-pyridyl | P.71 | IA-1850 | 3-pyridyl | P.71 |
| IA-1851 | 4-pyridyl | P.71 | IA-1852 | P.1 | P.71 |
| IA-1853 | P.2 | P.71 | IA-1854 | P.3 | P.71 |
| IA-1855 | P.6 | P.71 | IA-1856 | P.7 | P.71 |
| IA-1857 | P.8 | P.71 | IA-1858 | P.20 | P.71 |
| IA-1859 | P.48 | P.71 | IA-1860 | P.49 | P.71 |
| IA-1861 | P.52 | P.71 | IA-1862 | P.53 | P.71 |
| IA-1863 | P.54 | P.71 | IA-1864 | P.66 | P.71 |
| IA-1865 | P.101 | P.71 | IA-1866 | P.102 | P.71 |
| IA-1867 | P.111 | P.71 | IA-1868 | P.112 | P.71 |
| IA-1869 | P.127 | P.71 | IA-1870 | P.128 | P.71 |
| IA-1871 | P.273 | P.71 | IA-1872 | P.274 | P.71 |
| IA-1873 | P.393 | P.71 | IA-1874 | P.394 | P.71 |
| IA-1875 | P.395 | P.71 | IA-1876 | P.397 | P.71 |
| IA-1877 | P.402 | P.71 | IA-1878 | P.403 | P.71 |
| IA-1879 | P.404 | P.71 | IA-1880 | P.406 | P.71 |
| IA-1881 | H | P.88 | IA-1882 | F | P.88 |
| IA-1883 | Cl | P.88 | IA-1884 | Br | P.88 |
| IA-1885 | CH₃ | P.88 | IA-1886 | CN | P.88 |
| IA-1887 | CH═CH₂ | P.88 | IA-1888 | CCH | P.88 |
| IA-1889 | 2-pyridyl | P.88 | IA-1890 | 3-pyridyl | P.88 |
| IA-1891 | 4-pyridyl | P.88 | IA-1892 | P.1 | P.88 |
| IA-1893 | P.2 | P.88 | IA-1894 | P.3 | P.88 |
| IA-1895 | P.6 | P.88 | IA-1896 | P.7 | P.88 |
| IA-1897 | P.8 | P.88 | IA-1898 | P.20 | P.88 |
| IA-1899 | P.48 | P.88 | IA-1900 | P.49 | P.88 |
| IA-1901 | P.52 | P.88 | IA-1902 | P.53 | P.88 |
| IA-1903 | P.54 | P.88 | IA-1904 | P.66 | P.88 |
| IA-1905 | P.101 | P.88 | IA-1906 | P.102 | P.88 |
| IA-1907 | P.111 | P.88 | IA-1908 | P.112 | P.88 |
| IA-1909 | P.127 | P.88 | IA-1910 | P.128 | P.88 |
| IA-1911 | P.273 | P.88 | IA-1912 | P.274 | P.88 |
| IA-1913 | P.393 | P.88 | IA-1914 | P.394 | P.88 |
| IA-1915 | P.395 | P.88 | IA-1916 | P.397 | P.88 |
| IA-1917 | P.402 | P.88 | IA-1918 | P.403 | P.88 |
| IA-1919 | P.404 | P.88 | IA-1920 | P.406 | P.88 |
| IA-1921 | H | P.95 | IA-1922 | F | P.95 |
| IA-1923 | Cl | P.95 | IA-1924 | Br | P.95 |
| IA-1925 | CH₃ | P.95 | IA-1926 | CN | P.95 |
| IA-1927 | CH═CH₂ | P.95 | IA-1928 | CCH | P.95 |
| IA-1929 | 2-pyridyl | P.95 | IA-1930 | 3-pyridyl | P.95 |
| IA-1931 | 4-pyridyl | P.95 | IA-1932 | P.1 | P.95 |
| IA-1933 | P.2 | P.95 | IA-1934 | P.3 | P.95 |
| IA-1935 | P.6 | P.95 | IA-1936 | P.7 | P.95 |
| IA-1937 | P.8 | P.95 | IA-1938 | P.20 | P.95 |
| IA-1939 | P.48 | P.95 | IA-1940 | P.49 | P.95 |
| IA-1941 | P.52 | P.95 | IA-1942 | P.53 | P.95 |
| IA-1943 | P.54 | P.95 | IA-1944 | P.66 | P.95 |
| IA-1945 | P.101 | P.95 | IA-1946 | P.102 | P.95 |
| IA-1947 | P.111 | P.95 | IA-1948 | P.112 | P.95 |
| IA-1949 | P.127 | P.95 | IA-1950 | P.128 | P.95 |
| IA-1951 | P.273 | P.95 | IA-1952 | P.274 | P.95 |
| IA-1953 | P.393 | P.95 | IA-1954 | P.394 | P.95 |
| IA-1955 | P.395 | P.95 | IA-1956 | P.397 | P.95 |
| IA-1957 | P.402 | P.95 | IA-1958 | P.403 | P.95 |
| IA-1959 | P.404 | P.95 | IA-1960 | P.406 | P.95 |
| IA-1961 | H | P.96 | IA-1962 | F | P.96 |
| IA-1963 | Cl | P.96 | IA-1964 | Br | P.96 |
| IA-1965 | CH₃ | P.96 | IA-1966 | CN | P.96 |
| IA-1967 | CH═CH₂ | P.96 | IA-1968 | CCH | P.96 |
| IA-1969 | 2-pyridyl | P.96 | IA-1970 | 3-pyridyl | P.96 |
| IA-1971 | 4-pyridyl | P.96 | IA-1972 | P.1 | P.96 |
| IA-1973 | P.2 | P.96 | IA-1974 | P.3 | P.96 |
| IA-1975 | P.6 | P.96 | IA-1976 | P.7 | P.96 |
| IA-1977 | P.8 | P.96 | IA-1978 | P.20 | P.96 |
| IA-1979 | P.48 | P.96 | IA-1980 | P.49 | P.96 |
| IA-1981 | P.52 | P.96 | IA-1982 | P.53 | P.96 |
| IA-1983 | P.54 | P.96 | IA-1984 | P.66 | P.96 |
| IA-1985 | P.101 | P.96 | IA-1986 | P.102 | P.96 |
| IA-1987 | P.111 | P.96 | IA-1988 | P.112 | P.96 |
| IA-1989 | P.127 | P.96 | IA-1990 | P.128 | P.96 |
| IA-1991 | P.273 | P.96 | IA-1992 | P.274 | P.96 |
| IA-1993 | P.393 | P.96 | IA-1994 | P.394 | P.96 |
| IA-1995 | P.395 | P.96 | IA-1996 | P.397 | P.96 |
| IA-1997 | P.402 | P.96 | IA-1998 | P.403 | P.96 |
| IA-1999 | P.404 | P.96 | IA-2000 | P.406 | P.96 |
| IA-2001 | H | P.97 | IA-2002 | F | P.97 |
| IA-2003 | Cl | P.97 | IA-2004 | Br | P.97 |
| IA-2005 | CH₃ | P.97 | IA-2006 | CN | P.97 |
| IA-2007 | CH═CH₂ | P.97 | IA-2008 | CCH | P.97 |
| IA-2009 | 2-pyridyl | P.97 | IA-2010 | 3-pyridyl | P.97 |
| IA-2011 | 4-pyridyl | P.97 | IA-2012 | P.1 | P.97 |
| IA-2013 | P.2 | P.97 | IA-2014 | P.3 | P.97 |
| IA-2015 | P.6 | P.97 | IA-2016 | P.7 | P.97 |
| IA-2017 | P.8 | P.97 | IA-2018 | P.20 | P.97 |
| IA-2019 | P.48 | P.97 | IA-2020 | P.49 | P.97 |
| IA-2021 | P.52 | P.97 | IA-2022 | P.53 | P.97 |
| IA-2023 | P.54 | P.97 | IA-2024 | P.66 | P.97 |
| IA-2025 | P.101 | P.97 | IA-2026 | P.102 | P.97 |
| IA-2027 | P.111 | P.97 | IA-2028 | P.112 | P.97 |
| IA-2029 | P.127 | P.97 | IA-2030 | P.128 | P.97 |
| IA-2031 | P.273 | P.97 | IA-2032 | P.274 | P.97 |
| IA-2033 | P.393 | P.97 | IA-2034 | P.394 | P.97 |
| IA-2035 | P.395 | P.97 | IA-2036 | P.397 | P.97 |
| IA-2037 | P.402 | P.97 | IA-2038 | P.403 | P.97 |
| IA-2039 | P.404 | P.97 | IA-2040 | P.406 | P.97 |
| IA-2041 | H | P.104 | IA-2042 | F | P.104 |
| IA-2043 | Cl | P.104 | IA-2044 | Br | P.104 |
| IA-2045 | CH₃ | P.104 | IA-2046 | CN | P.104 |
| IA-2047 | CH═CH₂ | P.104 | IA-2048 | CCH | P.104 |
| IA-2049 | 2-pyridyl | P.104 | IA-2050 | 3-pyridyl | P.104 |
| IA-2051 | 4-pyridyl | P.104 | IA-2052 | P.1 | P.104 |
| IA-2053 | P.2 | P.104 | IA-2054 | P.3 | P.104 |
| IA-2055 | P.6 | P.104 | IA-2056 | P.7 | P.104 |
| IA-2057 | P.8 | P.104 | IA-2058 | P.20 | P.104 |
| IA-2059 | P.48 | P.104 | IA-2060 | P.49 | P.104 |
| IA-2061 | P.52 | P.104 | IA-2062 | P.53 | P.104 |
| IA-2063 | P.54 | P.104 | IA-2064 | P.66 | P.104 |
| IA-2065 | P.101 | P.104 | IA-2066 | P.102 | P.104 |
| IA-2067 | P.111 | P.104 | IA-2068 | P.112 | P.104 |
| IA-2069 | P.127 | P.104 | IA-2070 | P.128 | P.104 |
| IA-2071 | P.273 | P.104 | IA-2072 | P.274 | P.104 |
| IA-2073 | P.393 | P.104 | IA-2074 | P.394 | P.104 |
| IA-2075 | P.395 | P.104 | IA-2076 | P.397 | P.104 |
| IA-2077 | P.402 | P.104 | IA-2078 | P.403 | P.104 |
| IA-2079 | P.404 | P.104 | IA-2080 | P.406 | P.104 |
| IA-2081 | H | P.105 | IA-2082 | F | P.105 |
| IA-2083 | Cl | P.105 | IA-2084 | Br | P.105 |
| IA-2085 | CH₃ | P.105 | IA-2086 | CN | P.105 |
| IA-2087 | CH═CH₂ | P.105 | IA-2088 | CCH | P.105 |
| IA-2089 | 2-pyridyl | P.105 | IA-2090 | 3-pyridyl | P.105 |
| IA-2091 | 4-pyridyl | P.105 | IA-2092 | P.1 | P.105 |
| IA-2093 | P.2 | P.105 | IA-2094 | P.3 | P.105 |
| IA-2095 | P.6 | P.105 | IA-2096 | P.7 | P.105 |
| IA-2097 | P.8 | P.105 | IA-2098 | P.20 | P.105 |
| IA-2099 | P.48 | P.105 | IA-2100 | P.49 | P.105 |
| IA-2101 | P.52 | P.105 | IA-2102 | P.53 | P.105 |
| IA-2103 | P.54 | P.105 | IA-2104 | P.66 | P.105 |
| IA-2105 | P.101 | P.105 | IA-2106 | P.102 | P.105 |
| IA-2107 | P.111 | P.105 | IA-2108 | P.112 | P.105 |
| IA-2109 | P.127 | P.105 | IA-2110 | P.128 | P.105 |
| IA-2111 | P.273 | P.105 | IA-2112 | P.274 | P.105 |
| IA-2113 | P.393 | P.105 | IA-2114 | P.394 | P.105 |
| IA-2115 | P.395 | P.105 | IA-2116 | P.397 | P.105 |
| IA-2117 | P.402 | P.105 | IA-2118 | P.403 | P.105 |
| IA-2119 | P.404 | P.105 | IA-2120 | P.406 | P.105 |
| IA-2121 | H | P.106 | IA-2122 | F | P.106 |
| IA-2123 | Cl | P.106 | IA-2124 | Br | P.106 |
| IA-2125 | CH₃ | P.106 | IA-2126 | CN | P.106 |
| IA-2127 | CH═CH₂ | P.106 | IA-2128 | CCH | P.106 |
| IA-2129 | 2-pyridyl | P.106 | IA-2130 | 3-pyridyl | P.106 |
| IA-2131 | 4-pyridyl | P.106 | IA-2132 | P.1 | P.106 |
| IA-2133 | P.2 | P.106 | IA-2134 | P.3 | P.106 |
| IA-2135 | P.6 | P.106 | IA-2136 | P.7 | P.106 |
| IA-2137 | P.8 | P.106 | IA-2138 | P.20 | P.106 |
| IA-2139 | P.48 | P.106 | IA-2140 | P.49 | P.106 |
| IA-2141 | P.52 | P.106 | IA-2142 | P.53 | P.106 |
| IA-2143 | P.54 | P.106 | IA-2144 | P.66 | P.106 |
| IA-2145 | P.101 | P.106 | IA-2146 | P.102 | P.106 |
| IA-2147 | P.111 | P.106 | IA-2148 | P.112 | P.106 |
| IA-2149 | P.127 | P.106 | IA-2150 | P.128 | P.106 |
| IA-2151 | P.273 | P.106 | IA-2152 | P.274 | P.106 |
| IA-2153 | P.393 | P.106 | IA-2154 | P.394 | P.106 |
| IA-2155 | P.395 | P.106 | IA-2156 | P.397 | P.106 |
| IA-2157 | P.402 | P.106 | IA-2158 | P.403 | P.106 |
| IA-2159 | P.404 | P.106 | IA-2160 | P.406 | P.106 |
| IA-2161 | H | P.122 | IA-2162 | F | P.122 |
| IA-2163 | Cl | P.122 | IA-2164 | Br | P.122 |
| IA-2165 | CH₃ | P.122 | IA-2166 | CN | P.122 |
| IA-2167 | CH═CH₂ | P.122 | IA-2168 | CCH | P.122 |
| IA-2169 | 2-pyridyl | P.122 | IA-2170 | 3-pyridyl | P.122 |
| IA-2171 | 4-pyridyl | P.122 | IA-2172 | P.1 | P.122 |
| IA-2173 | P.2 | P.122 | IA-2174 | P.3 | P.122 |
| IA-2175 | P.6 | P.122 | IA-2176 | P.7 | P.122 |
| IA-2177 | P.8 | P.122 | IA-2178 | P.20 | P.122 |
| IA-2179 | P.48 | P.122 | IA-2180 | P.49 | P.122 |
| IA-2181 | P.52 | P.122 | IA-2182 | P.53 | P.122 |
| IA-2183 | P.54 | P.122 | IA-2184 | P.66 | P.122 |
| IA-2185 | P.101 | P.122 | IA-2186 | P.102 | P.122 |
| IA-2187 | P.111 | P.122 | IA-2188 | P.112 | P.122 |
| IA-2189 | P.127 | P.122 | IA-2190 | P.128 | P.122 |
| IA-2191 | P.273 | P.122 | IA-2192 | P.274 | P.122 |
| IA-2193 | P.393 | P.122 | IA-2194 | P.394 | P.122 |
| IA-2195 | P.395 | P.122 | IA-2196 | P.397 | P.122 |
| IA-2197 | P.402 | P.122 | IA-2198 | P.403 | P.122 |
| IA-2199 | P.404 | P.122 | IA-2200 | P.406 | P.122 |
| IA-2201 | H | P.123 | IA-2202 | F | P.123 |
| IA-2203 | Cl | P.123 | IA-2204 | Br | P.123 |
| IA-2205 | CH₃ | P.123 | IA-2206 | CN | P.123 |
| IA-2207 | CH═CH₂ | P.123 | IA-2208 | CCH | P.123 |
| IA-2209 | 2-pyridyl | P.123 | IA-2210 | 3-pyridyl | P.123 |
| IA-2211 | 4-pyridyl | P.123 | IA-2212 | P.1 | P.123 |
| IA-2213 | P.2 | P.123 | IA-2214 | P.3 | P.123 |
| IA-2215 | P.6 | P.123 | IA-2216 | P.7 | P.123 |
| IA-2217 | P.8 | P.123 | IA-2218 | P.20 | P.123 |
| IA-2219 | P.48 | P.123 | IA-2220 | P.49 | P.123 |
| IA-2221 | P.52 | P.123 | IA-2222 | P.53 | P.123 |
| IA-2223 | P.54 | P.123 | IA-2224 | P.66 | P.123 |
| IA-2225 | P.101 | P.123 | IA-2226 | P.102 | P.123 |
| IA-2227 | P.111 | P.123 | IA-2228 | P.112 | P.123 |
| IA-2229 | P.127 | P.123 | IA-2230 | P.128 | P.123 |
| IA-2231 | P.273 | P.123 | IA-2232 | P.274 | P.123 |
| IA-2233 | P.393 | P.123 | IA-2234 | P.394 | P.123 |
| IA-2235 | P.395 | P.123 | IA-2236 | P.397 | P.123 |
| IA-2237 | P.402 | P.123 | IA-2238 | P.403 | P.123 |
| IA-2239 | P.404 | P.123 | IA-2240 | P.406 | P.123 |
| IA-2241 | H | P.124 | IA-2242 | F | P.124 |
| IA-2243 | Cl | P.124 | IA-2244 | Br | P.124 |
| IA-2245 | CH₃ | P.124 | IA-2246 | CN | P.124 |
| IA-2247 | CH═CH₂ | P.124 | IA-2248 | CCH | P.124 |
| IA-2249 | 2-pyridyl | P.124 | IA-2250 | 3-pyridyl | P.124 |
| IA-2251 | 4-pyridyl | P.124 | IA-2252 | P.1 | P.124 |
| IA-2253 | P.2 | P.124 | IA-2254 | P.3 | P.124 |
| IA-2255 | P.6 | P.124 | IA-2256 | P.7 | P.124 |
| IA-2257 | P.8 | P.124 | IA-2258 | P.20 | P.124 |
| IA-2259 | P.48 | P.124 | IA-2260 | P.49 | P.124 |
| IA-2261 | P.52 | P.124 | IA-2262 | P.53 | P.124 |
| IA-2263 | P.54 | P.124 | IA-2264 | P.66 | P.124 |
| IA-2265 | P.101 | P.124 | IA-2266 | P.102 | P.124 |
| IA-2267 | P.111 | P.124 | IA-2268 | P.112 | P.124 |
| IA-2269 | P.127 | P.124 | IA-2270 | P.128 | P.124 |
| IA-2271 | P.273 | P.124 | IA-2272 | P.274 | P.124 |
| IA-2273 | P.393 | P.124 | IA-2274 | P.394 | P.124 |
| IA-2275 | P.395 | P.124 | IA-2276 | P.397 | P.124 |
| IA-2277 | P.402 | P.124 | IA-2278 | P.403 | P.124 |
| IA-2279 | P.404 | P.124 | IA-2280 | P.406 | P.124 |
| IA-2281 | CC—(CH₂)₂CH₃ | CH₃ | IA-2282 | CC- | CH₃ |
| | | | | cyclo- | |
| | | | | C₅H₉ | |
| IA-2283 | CC—(CH₂)₂CH₃ | CH₂CH₃ | IA-2284 | CC- | CH₂CH₃ |
| | | | | cyclo- | |
| | | | | C₅H₉ | |
| IA-2285 | CC—(CH₂)₂CH₃ | CH₂CH₂CH₃ | IA-2286 | CC- | CH₂CH₂CH₃ |
| | | | | cyclo- | |
| | | | | C₅H₉ | |
| IA-2287 | CC—(CH₂)₂CH₃ | (CH₂)₄CH₃ | IA-2288 | CC- | (CH₂)₄CH₃ |
| | | | | cyclo- | |
| | | | | C₅H₉ | |
| IA-2289 | CC—(CH₂)₂CH₃ | CH(CH₃)₂ | IA-2290 | CC- | CH(CH₃)₂ |
| | | | | cyclo- | |
| | | | | C₅H₉ | |
| IA-2291 | CC—(CH₂)₂CH₃ | CH₂CH(CH₃)₂ | IA-2292 | CC- | CH₂CH(CH₃)₂ |
| | | | | cyclo- | |
| | | | | C₅H₉ | |
| IA-2293 | CC—(CH₂)₂CH₃ | C(CH₃)₃ | IA-2294 | CC- | C(CH₃)₃ |
| | | | | cyclo- | |
| | | | | C₅H₉ | |
| IA-2295 | CC—(CH₂)₂CH₃ | CH═CH₂ | IA-2296 | CC- | CH═CH₂ |
| | | | | cyclo- | |
| | | | | C₅H₉ | |
| IA-2297 | CC—(CH₂)₂CH₃ | CCH | IA-2298 | CC- | CCH |
| | | | | cyclo- | |
| | | | | C₅H₉ | |
| IA-2299 | CC—(CH₂)₂CH₃ | CH₂OCH₃ | IA-2300 | CC- | CH₂OCH₃ |
| | | | | cyclo- | |
| | | | | C₅H₉ | |
| IA-2301 | CC—(CH₂)₂CH₃ | CH₂Cl | IA-2302 | CC- | CH₂Cl |
| | | | | cyclo- | |
| | | | | C₅H₉ | |
| IA-2303 | CC—(CH₂)₂CH₃ | CH₂SCH₃ | IA-2304 | CC- | CH₂SCH₃ |
| | | | | cyclo- | |
| | | | | C₅H₉ | |
| IA-2305 | CC—(CH₂)₂CH₃ | CH₂C₆H₅ | IA-2306 | CC- | CH₂C₆H₅ |
| | | | | cyclo- | |
| | | | | C₅H₉ | |
| IA-2307 | CC—(CH₂)₂CH₃ | cyclo-C₃H₅ | IA-2308 | CC- | cyclo-C₃H₅ |
| | | | | cyclo- | |
| | | | | C₅H₉ | |
| IA-2309 | CC—(CH₂)₂CH₃ | cyclo-C₆H₁₁ | IA-2310 | CC- | cyclo-C₆H₁₁ |
| | | | | cyclo- | |
| | | | | C₅H₉ | |
| IA-2311 | CC—(CH₂)₂CH₃ | 3-tetrahydrofuranyl | IA-2312 | CC- | 3-tetrahydrofuranyl |
| | | | | cyclo- | |
| | | | | C₅H₉ | |
| IA-2313 | CC—(CH₂)₂CH₃ | 5-oxo- | IA-2314 | CC- | 5-oxo- |
| | | tetrahydrofuran-3-yl | | cyclo- | tetrahydrofuran-3-yl |
| | | | | C₅H₉ | |
| IA-2315 | CC—(CH₂)₂CH₃ | 3-tetrahydropyranyl | IA-2316 | CC- | 3-tetrahydropyranyl |
| | | | | cyclo- | |
| | | | | C₅H₉ | |
| IA-2317 | CC—(CH₂)₂CH₃ | 4-pyridyl | IA-2318 | CC- | 4-pyridyl |
| | | | | cyclo- | |
| | | | | C₅H₉ | |
| IA-2319 | CC—(CH₂)₂CH₃ | 3-thiophenyl | IA-2320 | CC- | 3-thiophenyl |
| | | | | cyclo- | |
| | | | | C₅H₉ | |
| IA-2321 | CC—(CH₂)₂CH₃ | 3-furanyl | IA-2322 | CC- | 3-furanyl |
| | | | | cyclo- | |
| | | | | C₅H₉ | |
| IA-2323 | CC—(CH₂)₂CH₃ | 5-CH₃-isoxazol-3-yl | IA-2324 | CC- | 5-CH₃-isoxazol-3-yl |
| | | | | cyclo- | |
| | | | | C₅H₉ | |
| IA-2325 | CC—(CH₂)₂CH₃ | 3-CH₃-isoxazol-5-yl | IA-2326 | CC- | 3-CH₃-isoxazol-5-yl |
| | | | | cyclo- | |
| | | | | C₅H₉ | |
| IA-2327 | CC—(CH₂)₂CH₃ | P.1 | IA-2328 | CC- | P.1 |
| | | | | cyclo- | |
| | | | | C₅H₉ | |
| IA-2329 | CC—(CH₂)₂CH₃ | P.2 | IA-2330 | CC- | P.2 |
| | | | | cyclo- | |
| | | | | C₅H₉ | |
| IA-2331 | CC—(CH₂)₂CH₃ | P.3 | IA-2332 | CC- | P.3 |
| | | | | cyclo- | |
| | | | | C₅H₉ | |
| IA-2333 | CC—(CH₂)₂CH₃ | P.6 | IA-2334 | CC- | P.6 |
| | | | | cyclo- | |
| | | | | C₅H₉ | |
| IA-2335 | CC—(CH₂)₂CH₃ | P.7 | IA-2336 | CC- | P.7 |
| | | | | cyclo- | |
| | | | | C₅H₉ | |
| IA-2337 | CC—(CH₂)₂CH₃ | P.8 | IA-2338 | CC- | P.8 |
| | | | | cyclo- | |
| | | | | C₅H₉ | |
| IA-2339 | CC—(CH₂)₂CH₃ | P.12 | IA-2340 | CC- | P.12 |
| | | | | cyclo- | |
| | | | | C₅H₉ | |
| IA-2341 | CC—(CH₂)₂CH₃ | P.14 | IA-2342 | CC- | P.14 |
| | | | | cyclo- | |
| | | | | C₅H₉ | |
| IA-2343 | CC—(CH₂)₂CH₃ | P.19 | IA-2344 | CC- | P.19 |
| | | | | cyclo- | |
| | | | | C₅H₉ | |
| IA-2345 | CC—(CH₂)₂CH₃ | P.20 | IA-2346 | CC- | P.20 |
| | | | | cyclo- | |
| | | | | C₅H₉ | |
| IA-2347 | CC—(CH₂)₂CH₃ | P.23 | IA-2348 | CC- | P.23 |
| | | | | cyclo- | |
| | | | | C₅H₉ | |
| IA-2349 | CC—(CH₂)₂CH₃ | P.25 | IA-2350 | CC- | P.25 |
| | | | | cyclo- | |
| | | | | C₅H₉ | |
| IA-2351 | CC—(CH₂)₂CH₃ | P.28 | IA-2352 | CC- | P.28 |
| | | | | cyclo- | |
| | | | | C₅H₉ | |
| IA-2353 | CC—(CH₂)₂CH₃ | P.33 | IA-2354 | CC- | P.33 |
| | | | | cyclo- | |
| | | | | C₅H₉ | |
| IA-2355 | CC—(CH₂)₂CH₃ | P.42 | IA-2356 | CC- | P.42 |
| | | | | cyclo- | |
| | | | | C₅H₉ | |
| IA-2357 | CC—(CH₂)₂CH₃ | P.45 | IA-2358 | CC- | P.45 |
| | | | | cyclo- | |
| | | | | C₅H₉ | |
| IA-2359 | CC—(CH₂)₂CH₃ | P.48 | IA-2360 | CC- | P.48 |
| | | | | cyclo- | |
| | | | | C₅H₉ | |
| IA-2361 | CC—(CH₂)₂CH₃ | P.49 | IA-2362 | CC- | P.49 |
| | | | | cyclo- | |
| | | | | C₅H₉ | |
| IA-2363 | CC—(CH₂)₂CH₃ | P.52 | IA-2364 | CC- | P.52 |
| | | | | cyclo- | |
| | | | | C₅H₉ | |
| IA-2365 | CC—(CH₂)₂CH₃ | P.53 | IA-2366 | CC- | P.53 |
| | | | | cyclo- | |
| | | | | C₅H₉ | |
| IA-2367 | CC—(CH₂)₂CH₃ | P.54 | IA-2368 | CC- | P.54 |
| | | | | cyclo- | |
| | | | | C₅H₉ | |
| IA-2369 | CC—(CH₂)₂CH₃ | P.65 | IA-2370 | CC- | P.65 |
| | | | | cyclo- | |
| | | | | C₅H₉ | |
| IA-2371 | CC—(CH₂)₂CH₃ | P.66 | IA-2372 | CC- | P.66 |
| | | | | cyclo- | |
| | | | | C₅H₉ | |
| IA-2373 | CC—(CH₂)₂CH₃ | P.71 | IA-2374 | CC- | P.71 |
| | | | | cyclo- | |
| | | | | C₅H₉ | |
| IA-2375 | CC—(CH₂)₂CH₃ | P.88 | IA-2376 | CC- | P.88 |
| | | | | cyclo- | |
| | | | | C₅H₉ | |
| IA-2377 | CC—(CH₂)₂CH₃ | P.95 | IA-2378 | CC- | P.95 |
| | | | | cyclo- | |
| | | | | C₅H₉ | |
| IA-2379 | CC—(CH₂)₂CH₃ | P.96 | IA-2380 | CC- | P.96 |
| | | | | cyclo- | |
| | | | | C₅H₉ | |
| IA-2381 | CC—(CH₂)₂CH₃ | P.97 | IA-2382 | CC- | P.97 |
| | | | | cyclo- | |
| | | | | C₅H₉ | |
| IA-2383 | CC—(CH₂)₂CH₃ | P.104 | IA-2384 | CC- | P.104 |
| | | | | cyclo- | |
| | | | | C₅H₉ | |
| IA-2385 | CC—(CH₂)₂CH₃ | P.105 | IA-2386 | CC- | P.105 |
| | | | | cyclo- | |
| | | | | C₅H₉ | |
| IA-2387 | CC—(CH₂)₂CH₃ | P.106 | IA-2388 | CC- | P.106 |
| | | | | cyclo- | |
| | | | | C₅H₉ | |
| IA-2389 | CC—(CH₂)₂CH₃ | P.122 | IA-2390 | CC- | P.122 |
| | | | | cyclo- | |
| | | | | C₅H₉ | |
| IA-2391 | CC—(CH₂)₂CH₃ | P.123 | IA-2392 | CC- | P.123 |
| | | | | cyclo- | |
| | | | | C₅H₉ | |
| IA-2393 | CC—(CH₂)₂CH₃ | P.124 | IA-2394 | CC- | P.124 |
| | | | | cyclo- | |
| | | | | C₅H₉ | |

With respect to their intended pesticidal use, the variables and indices of the compounds II have the same preferred meanings as described above for compounds 1.

The compounds I-1 wherein y is zero of the present invention can be prepared by amidation of the corresponding amines II

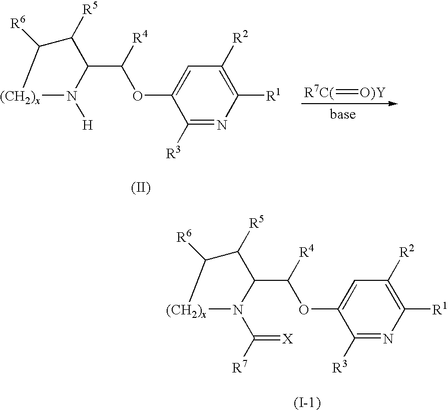

with an activated carboxylic acid derivative R⁷(═O)Y in the presence of a base, wherein the variables in these compounds are as defined at the outset for compounds I and Y is OH or a suitable leaving group as chlorine or bromine, OR^{d}, OC(═O)R^{e}, imidazole with R^{d} being C₁-C₆-alkyl, preferably methyl or ethyl, or N-hydroxybenzotriazole, and R^{e} is C₁-C₆-alkyl or phenyl, preferably methyl, ethyl, sec-butyl, isobutyl, or 2,4,6-trimethylphenyl.

Various reagents and conditions are known to the artisan from literature, e.g. from Hoben-Weyl, Methoden der Organischen Chemie E5 Teil 2, Thieme Verlag Stuttgart 1985, or Contemp. Organic Synthesis, 1995, 2(4), p. 269.

The carboxylic acid R⁷C(═O)OH may be activated by a carbodiimide e.g. dicyclohexylcarbodiimide, di-isopropylcarbodiimide, N-(3-dimethylaminopropyl)-N′-ethyl carbodiimide or its hydrogen chloride salt, or their polymeric immobilized derivatives such as N-(3-dimethylaminopropyl)-N′-ethyl carbodiimide-polystyrene. The activating reagent can also be an Uronium salt like O-(benzotriazol-1-yl)-N,N,N′,N′-tetramethyluronium tetrafluoroborate (TBTU) or hexafluorophosphate (HBTU) or their respective polymeric forms.

Detailed references for activation by carbodiimide formation are given in: Chem. Rev. 62, 1967, p. 12, or J. Org. Chem. 26, 1961, p. 2525, or Chem. Ber. 103, 1970, p. 788 and cited literature. For uronium salt formation, Tetrahedron Lett. 1989, 30 p. 1927, Helv. Chim. Acta 1991, 74, p. 617, or Tetrahedron Lett. 2000, 41, p. 2463 are given as references.

The carboxyclic acids R⁷C(═O)OH are commercially available.

The carboxylic acid R⁷C(═O)OH is used in the range of 1 to 3, preferably 0.8 to 0.99 molar equivalents of compound I. Their carbodiimides or uronium salts are used in the range of 1.0 to 1.5 equivalents. In some cases it is favorable to use 1.5 to 5 equivalent to drive reaction to complete consumption of the acid and the amine.

The base is selected from organic bases like triethylamine, diisopropylamine, N-methyl morpholine, N-methylpiperidine, N-ethyl morpholine, N-ethylpiperidine or pyridine. The base is used in a stochiometry of 0.8 to 10 molar equivalents of compounds I and can also be used as a solvent.

Optionally, and as described in the literature cited above, the activated carboxylic acid can be reacted with the amine II in the presence of an active ester forming reagent like hydroxybenzotriaole.

The activated carboxylic acid R⁷C(═O)OH can be reacted with the amine 11 with or without the addition of acylation acceleration agents like 4-dimethyaminopyridine.

Optionally, the acylating agent can be an acyl bromide or chloride or a symmetrical or unsymmetrical anhydride or an imidazolide of R⁷C(═O)OH.

Acid chlorides R⁷C(═O)Cl are commercially available or can be prepared from the carboxylic acids by methods known to those skilled in the art e.g. by treatment with SOCl₂ as described in Houben-Weyl, Methoden der Organischen Chemie E5, Teil 1, Thieme Verlag Stuttgart 1985. R⁷C(═O)Br, the activated acid is preferably prepared in situ by use PyBroP (Brom-tris-pyrrolidinophosphonium hexafluorophosphate) as described in Tetrahedron, 1991, 47(2), pp. 259-70 or Tetrahedron Letters, 1991, 32(17), pp. 1967-70.

Symmetrical or unsymmetrical anhydrides of R⁷C(═O)OH are prepared preferably prior to the amidation either by use of the above mentioned carbodiimide in case of symmetrical anhydrides or by treating a salt of the carboxylic acid with a chloroformate or with trichlorobenzoate, all as described in the art, e.g. in Houben Weyl, Methoden der Organischen Chemie E5, Teil 1, Thieme Verlag Stuttgart, 1985. Unsymmetrical anhydrides can also be prepared in situ by the use of EEDQ (2-ethoxy-1-ethoxycarbonyl-1,2-dihydrochinoline) or II DQ (2-isopropoxy-1-isopropoxycarbonyl-1,2-dihydrochinoline) or their polymeric immobilized derivatives. Details might be taken from J. Chem. Soc. Comm. 1972, p. 942 or J. Am. Chem. SOC. 90, 1968, p. 1651.

The solvent is an inert organic solvent like a halogenated or aromatic hydrocarbon, e.g. methylenechloride or chlorobenzene, an ether e.g. tetrahydrofuran, ethyleneglykoldimethylether, methyl tertbutylether, or dimethylformamid, N-methylpyrrolidone, toluene, or mixtures of these solvents.

The acylimidazolids of R⁷C(═O)OH can be prepared according to “Newer methods of preparative organic chemistry”, Vol 5, Verlag Chemie, 1967, p. 74.

The reaction temperature ranges from −20° C. to 100° C., preferably from 0° C. to 30° C.

The activation of the carboxyl acid R⁷C(═O)OH can be accomplished in situ or in an additional reaction step before the amidation.

In all cases, the acylating agent is used in a stoechiometry of 1 to 3 molar equivalents of compound II, in some cases it is favorable to use a substoechiometric amount of activated acid in the range of 0.8 to 0.99 equivalents to simplify the workup and purification of the product.

The base used is selected from organic bases like triethylamine, diisopropylamine, N-methyl morpholine, N-methylpiperidine, N-ethyl morpholine, N-ethylpiperidine or pyridine. The stoechiometry is 0.8 to 10 equivalents of base, in some cases it can be favorable to use the base as solvent.

The compounds of formula II are known from the literature (U.S. Pat. No. 5,629,325, U.S. Pat. No. 6,127,386, U.S. Pat. No. 6,437,138, WO 94 08992, WO 96/40682, WO 97/46554, WO 98/25920, WO 99/32480, WO 05/000806), or they can be prepared according to the procedures given therein as depicted below,

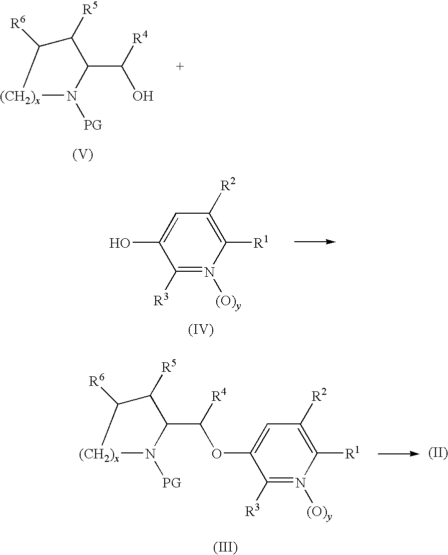

wherein PG is a protection group.

The intermediates III can also be obtained as described in this literature. Additional methods for the synthesis of substituted intermediates III, especially those wherein x is zero, are given in US 2005/043248, US 2005/012320, WO 04/071454, Tetrahedron Lett. 2004, 45 (17), p. 3555, Tetrahedron Lett. 1997, 38 (22), p. 3813, or WO 04/071454.

Hydroxypyridines IV are commercially available or can be synthesized according to the literature cited for the preparation of compounds II.

Optionally, compounds I-1 or I (or III) wherein R¹ and R² are halogen can be converted into further derivatives I-1 or I (or III) by substitution of R¹ and/or R² with nucleophiles like amines (Chem. Ber. 1969, 102, p. 1161) thiols (Tetrahedron, 1985, 41, p. 1373, Tetrahedron, 1983, p. 4153), alkoxides (Tetrahedron, 1992, 48, p. 3633), a boronic acid under Suzuki conditions (J. Org. Chem. 67, 2002, p. 5588, U.S. Pat. No. 6,127,386,U.S. Pat. No. 6,437,138), a tinalkyl or tinalkenyl under Stille conditions (Monatshefte Chem. 1995, 126, p. 805) an acetylene under Sonogashira conditions (Tetrahedron Asym. 12, 2001 p. 1121, J. Org. Chem. 68, 2003, p. 1571, J. Med. Chem. 2005, 48, p. 1721, U.S. Pat. No. 6,127,386) or a cyanide source (J. Chem. Res., Synop. 2003, 12, p. 814, Eur J. Chem. 2003, 9 (8), pp. 1828.

Compounds I-1 or I (or III) wherein R² is a ring Q.1-Q.31 can be obtained according to methods described in the art, for example in Joule, Mills, “Heterocyclic Chemistry”, Chapman+Hill 2000; Katritzky, Rees “Comprehensive Heterocyclic Chemistry”, Vol. 1-8, Pergamon Press 1984; “The Chemistry of Heterocyclic Compounds”. Wiley, Vol. 14 (1974), Vol. 29 (1974), 34 (1979), 37 (1981), 41 (1982), 49 (1991) oder Houben Weyl, “The Science of Synthesis”, Vol 9-22, Thieme 2005; or in references cited therein.

Compounds of the formula I-1 can be transferred into compounds I wherein y is 1 by oxidation according to procedures known in the art, e.g. in Recl. Trav. Pasy-Bas, 1957, 76, p. 58, Synth. Comm. 2000, 30(8), p. 1529, J. Het. Chem. 1996, 33 (4), p. 1051.

After completion of the reaction, the compound of formula (I) can be isolated by employing conventional methods such as adding the reaction mixture to water, extracting with an organic solvent, concentrating the extract an the like. The isolated compound (I) can be purified by a technique such as chromatography, recrystallization and the like, if necessary.

If individual compounds I or II are not obtainable by the routes described above, they can be prepared by derivatization of other compounds I or II or by customary modifications of the synthesis routes described.

The preparation of the compounds of formula I or II may lead to them being obtained as isomer mixtures (stereoisomers, enantiomers). If desired, these can be resolved by the methods customary for this purpose, such as crystallization or chromatography, also on optically active adsorbate, to give the pure isomers.

Agronomically acceptable salts of the compounds I or II can be formed in a customary manner, e.g. by reaction with an acid of the anion in question.

The compounds of the formula I or II are especially suitable for efficiently combating the following pests:

insects from the order of the lepidopterans (Lepidoptera), for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Cheimatobia brumata, Choristoneura fumiferana, Choristoneura occidentalis, Cirphis unipuncta, Cydia pomonella, Dendrolimus pini, Diaphania nitidalis, Diafraea grandiosella, Earias insulana, Elasmopalpus lignosellus, Eupoecilia ambiguella, Evetria bouiana, Feltia subterranea, Galleria mellonella, Grapholitha funebrana, Grapholitha molesta, Hellothis armigera, Heliothis virescens, Heliothis zea, Hellula undalis, Hibernia defoliaria, Hyphanfria cunea, Hyponomeuta malinellus, Keiferia lycopersicella, Lambdina fiscellaria, Laphygma exigua, Leucoptera coffeella, Leucoptera scitella, Lithocolletis blancardella, Lobesia botrana, Loxostege sticticalis, Lymanfria dispar, Lymantria monacha, Lyonetia clerkella, Malacosoma neustria, Mamestra brassicae, Orgyia pseudotsugata, Ostrinia nubilalis, Panolis flammea, Pectinophora gossypiella, Peridroma saucia, Phalera bucephala, Phthorimaea operculella, Phyllocnistis citrella, Pieris brassicae, Plathypena scabra, Plutella xylostella, Pseudoplusia includens, Rhyacionia frustrana, Scrobipalpula absoluta, Sltotroga cerealella, Sparganothis pillerinana, Spodoptera frugiperda, Spodoptera liftoralls, Spodoptera litura, Thaumatopoea pityocampa, Torfrix viridana, Trichoplusia ni and Zeiraphera canadensis,
beetles (Coleoptera), for example Agrilus sinuatus, Agriotes lineatus, Agriotes obscurus, Amphimallus solstitialis, Anisandrus dispar, Anthonomus grandis, Anthonomus pomorum, Aphthona euphorndae, Athous haemorrhoidalis, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Cetonia aurata, Ceuthorrhynchus assimilis, Ceuthorrhynchus napi, Chaetocnema tibialis, Conoderus vespertinus, Crioceris asparagi, Ctenicera ssp., Diabrotica longlcornlis, Diabrotica semipunctata, Diabrotica 12-punctata Diabrotica speciosa, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Ortiorrhynchus sulcatus, Ot/orrhynchus ovatus, Phaedon cochleariae, Phyllobius pyri, Phyllotreta chrysocephala, Phyllophaga sp., Phyllopertha horticola, Phyllotreta nemorum, Phyllotreta striolata, Popillia japonica, Sitona lineatus and Sitophilus granaria,
flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Ceratitis capitata, Chrysomya bezziana, Chrysomya hominivorax, Chrysomya macellaria, Chrysops discalis, Chrysops silacea, Chrysops atlanticus, Cochliomyia hominivorax, Contarinia sorghicola Cordylobia anthropophaga, Culicoides furens, Culex pipiens, Culex nigripalpus, Culex quinquefasciatus, Culex tarsalis, Culiseta inornata, Culiseta melanura, Dacus cucurbitae, Dacus oleae, Dasineura brassicae, Della antique, Della coarctata, Delia platura, Delia radicum, Dermatobia hominis, Fannia canicularis, Geomyza Tripunctata, Gasterophilus intestinalis, Glossina morsitans, Glossina palpalis, Glossina fuscipes, Glossina tachinoides, Haematobia irritans, Haplodiplosis equestris, Hippelates spp., Hylemyia platura, Hypoderma lineata, Leptoconops torrens, Liriomyza sativae, Liriomyza trifolii, Lucilia caprina, Lucilia cuprina, Lucilia sericata, Lycoria pectoralis, Mansonia titillanus, Mayetiola destructor, Musca domestica, Muscina stabulans, Oestrus ovis, Opomyza florum, Oscinella frit, Pegomya hysocyami, Phorbia antiqua, Phorbia brassicae, Phorbia coarctata, Phlebotomus argentipes, Psorophora columbiae, Psila rosae, Psorophora discolor, Prosimulium mixtum, Rhagoletis cerasi, Rhagoletis pomonella, Sarcophaga haemorrhoidalis, Sarcophaga sp., Simullum vittatum, Stomoxys calcitrans, Tabanus bovinus, Tabanus atratus, Tabanus lineola, and Tabanus similis, Tipula oleracea, and Tipula paludosa
thrips (Thysanoptera), e.g. Dichromothrips corbetti, Dichromothrips ssp, Frankliniella fusca, Frankliniella occidentals, Frankliniella tritici, Scirtothrips citri, Thrips oryzae, Thrips palmi and Thrips tabaci,
termites (Isoptera), e.g. Calotermes flavicollis, Leucotermes flavipes, Heterotermes aureus, Reticulitermes flavipes, Retculiltermes virginicus, Reticuitermes lucifugus, Termes natalensis, and Coptotermes formosanus,
cockroaches (Blattaria-Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis,
true bugs (Hemiptera), e.g. Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis, Thyanta perditor, Acyrthosiphon onobrychis, Adelges laricis, Aphidula nasturti, Aphis fabae, Aphis forbesi, Aphis pomi, Aphis gossypi, Aphis grossulariae, Aphis schneideri, Aphis spiraecola, Aphis sambuci, Acyrthosiphon pisum, Aulacorthum solani, Bemisia argentifolii, Brachycaudus cardui, Brachycaudus helichrysi, Brachycaudus persicae, Brachycaudus prunicola, Brevicoryne brassicae, Capitophorus horni, Cerosipha gossypli, Chaetosiphon fragaefolli, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus/actucae, Macrosiphum avenae, Macrosiphum euphorbiae, Macrosiphon rosae, Megoura viciae, Melanaphis pyrarius, Metopolophium dirhodum, Myzus persicae, Myzus ascalonicus, Myzus cerasi, Myzus varians, Nasonovia ribis-nigri Nilaparvata lugens, Pemphigus bursarius, Perkinsiella saccharicida, Phorodon humuli, Psylla mali, Psylla pir, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Trialeurodes vaporariorum, Toxoptera aurantiiand, Viteus viitfolii, Cimex lectularius, Cimex hemipterus, Reduvius senilis, Triatoma spp., and Arilus critatus.
ants, bees, wasps, sawflies (Hymenoptera), e.g. Athalia rosae, Atta cephalotes, Atta capiguara, Atta cephalotes, Atta laevigata, Atta robusta, Atta sexdens, Atta texana, Crematogaster spp., Hoplocampa minuta, Hoplocampa testudinea, Monomorium pharaonis, Solenopsis geminata, Solenopsis invicta, Solenopsis richteri, Solenopsis xyloni, Pogonomyrmex barbatus, Pogonomyrmex califormicus, Pheidole megacephala, Dasymutilla occidentalis, Bombus spp. Vespula squamosa, Paravespula vulgaris, Paravespula pennsylvanica, Paravespula german/ca, Dollchovespula maculata, Vespa crabro, Polistes rubiginosa, Camponotus floridanus, and Linepithema humile,
crickets, grasshoppers, locusts (Orthoptera), e.g. Acheta domestica, Gryllotalpa gryllotalpa, Locusta migratora, Melanoplus bivittatus, Melanoplus femurrubrum, Melanoplus mexicanus, Melanoplus sanguinipes, Melanoplus spretus, Nomadacris septemfasciata, Schistocerca americana, Schistocerca gregaria, Dociostaurus maroccanus, Tachycines asynamorus, Oedaleus senegalensis, Zonozerus variegatus, Hieroglyphus daganensis, Kraussaria angulifera, Calliptamus italicus, Chortoicetes terminifera, and Locustana pardalina,
Arachnoidea, such as arachnids (Acarina), e.g. of the families Argasidae, Ixodidae and Sarcoptidae, such as Amblyomma americanum, Amblyomma variegatum, Ambryomma maculatum, Argas persicus, Boophilus annulatus, Boophilus decoloratus, Boophilus microplus, Dermacentor silvarum, Dermacentor andersoni, Dermacentor variabilis, Hyalomma truncatum, Ixodes ricinus, Ixodes rubicundus, Ixodes scapulars, Ixodes holocyclus, Ixodes pacificus, Ornithodorus moubata, Ornithodorus hermsi, Ornithodorus turicata, Ornithonyssus bacoti, Otobius megnini, Dermanyssus gallinae, Psoroptes ovis, Rhipicephalus sanguineus, Rhipicephalus append/culatus, Rhipicephalus evertsi, Sarcoptes scabiei, and Eriophyidae spp. such as Aculus schlechtendali, Phyllocoptrata oleivora and Eriophyes sheldoni, Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and Oligonychus pratensis, Araneida, e.g. Latrodectus mactans, and Loxosceles reclusa,
fleas (Siphonaptera), e.g. Ctenocephalides felis, Ctenocephalides canis, Xenopsylla cheopis, Pulex irritans, Tunga penetrans, and Nosopsyllus fasciatus,
silverfish, firebrat (Thysanura), e.g. Lepisma saccharina and Thermobia domestica,
centipedes (Chilopoda), e.g. Scutigera coleoptrata,
millipedes (Diplopoda), e.g. Narceus spp.,
Earwigs (Dermaptera), e.g. forficula auricularia,
lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus,
Plant parasitic nematodes such as root-knot nematodes, Meloidogyne arenaria, Meloidogyne chitwoodi, Meloidogyne exigua, Meloidogyne hapla, Meloidogyne incognita, Meloidogyne javanica and other Meloidogyne species; cyst nematodes, Globodera rostochiensis, Globodera pallida, Globodera tabacum and other Globodera species, Heterodera avenae, Heterodera glycines, Heterodera schachtii, Heterodera trifolii, and other Heterodera species, seed gall nematodes, Anguina funesta, Anguina tritici and other Anguina species, stem and foliar nematodes, Aphelenchoides besseyi, Aphelenchoides fragariae, Aphelenchoides ritzemabosi and other Aphelenchoides species, sting nematodes, Belonolaimus longicaudatus and other Belonolaimus species, pine nematodes, Bursaphelenchus xylophilus and other Bursaphelenchus species, ring nematodes, Criconema species, Criconemella species, Criconemoides species, and Mesocriconema species; stem and bulb nematodes, Ditylenchus destructor, Ditylenchus dipsaci, Ditylenchus myceliophagus and other Ditylenchus species; awl nematodes, Dolichodorus species; spiral nematodes, Helicotylenchus dihystera, Helicotylenchus multicinctus and other Helicotylenchus species, Rotylenchus robustus and other Rotylenchus species, sheath nematodes, Hemicycliophora species and Hemicriconemoides species; Hirshmanniella species; lance nematodes, Hoplolaimus Columbus, Hoplolaimus galeatus and other Hoplolaimus species, false root-knot nematodes, Nacobbus aberrans and other Nacobbus species; needle nematodes, Longidorus elongates and other Longidorus species; pin nematodes, Paratylenchus species; lesion nematodes, Pratylenchus brachyurus, Pratylenchus coffeae, Pratylenchus curvitatus, Pratylenchus goodeyi, Pratylencus neglectus, Pratylenchus penetrans, Pratylenchus scribneri, Pratylenchus vulnus, Pratylenchus zeae and other Pratylenchus species; Radinaphelenchus cocophilus and other Radinaphelenchus species, burrowing nematodes, Radopholus similis and other Radopholus species; reniform nematodes, Rotylenchulus reniformis and other Rotylenchulus species; Scutellonema species; stubby root nematodes, Trichodorus primitivus and other Trichodorus species; Paratrichodorus minor and other Paratrichodorus species, stunt nematodes, Tylenchorhynchus clajtoni, Tylenchorhynchus dubius and other Tylenchorhynchus species and Merlinius species; citrus nematodes, Tylenchulus semipenetrans and other Tylenchulus species; dagger nematodes, Xiphinema americanum, Xiphinema index, Xiphinema diversicaudatum and other Xiphinema species, and other plant parasitic nematode species.

The formulations are prepared in a known manner (see e.g. for review U.S. Pat. No. 3,060,084, EP-A 707 445 (for liquid concentrates), Browning, “Agglomeration”, Chemical Engineering, Dec. 4, 1967, 147-48, Perry's Chemical Engineer's Handbook, 4th Ed., McGraw-Hill, New York, 1963, pages 8-57 and et seq. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman, Weed Control as a Science, John Wiley and Sons, Inc., New York, 1961, Hance et al., Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2. D. A. Knowles, Chemistry and Technology of Agrochemical Formulations, Kluwer Academic Publishers, Dordrecht, 1998 (ISBN 0-7515-0443-8), for example by extending the active compound with auxiliaries suitable for the formulation of agrochemicals, such as solvents and/or carriers, if desired emulsifiers, surfactants and dispersants, preservatives, anti-foaming agents, anti-freezing agents, for seed treatment formulation also optionally colorants and binders.

Examples of suitable solvents are water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used.

Examples of suitable carriers are ground natural minerals (for example kaolins, clays, talc, chalk) and ground synthetic minerals (for example highly disperse silica, silicates).

Suitable emulsifiers are nonionic and anionic emulsifiers (for example polyoxyethylene fatty alcohol ethers, alkylsulfonates and arylsulfonates).

Examples of dispersants are lignin-sulfite waste liquors and methylcellulose.

Suitable surfactants used are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ether, ethoxylated isooctylphenol, octylphenol, nonylphenol, alkylphenol polyglycol ethers, tributylphenyl polyglycol ether, tristearylphenyl polyglycol ether, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, highly polar solvents, for example dimethyl sulfoxide, N-methylpyrrolidone or water.

Also anti-freezing agents such as glycerin, ethylene glycol, propylene glycol and bactericides such as can be added to the formulation.

Suitable antifoaming agents are for example antifoaming agents based on silicon or magnesium stearate.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound(s). In this case, the active compound(s) are employed in a purity of from 90% to 100% by weight, preferably 95% to 100% by weight (according to NMR spectrum).

The compounds of formula I or II can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; they are intended to ensure in each case the finest possible distribution of the active compound(s) according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. However, it is also possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1% per weight.

The active compound(s) may also be used successfully in the ultra-low-volume process (ULV), it being possible to apply formulations comprising over 95% by weight of active compound, or even to apply the active compound without additives.

The following are examples of formulations: 1. Products for dilution with water for foliar applications. For seed treatment purposes, such products may be applied to the seed diluted or undiluted.

### A) Water-Soluable Concentrates (SL, LS)

10 parts by weight of the active compound(s) are dissolved in 90 parts by weight of water or a water-soluble solvent. As an alternative, wetters or other auxiliaries are added. The active compound(s) dissolves upon dilution with water, whereby a formulation with 10% (w/w) of active compound(s) is obtained.

### B) Dispersible Concentrates (DC)

20 parts by weight of the active compound(s) are dissolved in 75 parts by weight of cyclohexanone with addition of 10 parts by weight of a dispersant, for example polyvinylpyrrolidone. Dilution with water gives a dispersion, whereby a formulation with 20% (w/w) of active compound(s) is obtained.

### C) Emulsifiable Concentrates (EC)

15 parts by weight of the active compound(s) are dissolved in 75 parts by weight of xylene with addition of calcium dodecylbenzenesulfonate and castor oil ethoxylate (in each case 5 parts by weight). Dilution with water gives an emulsion, whereby a formulation with 15% (w/w) of active compound(s) is obtained.

### D) Emulsions (EW, EO, ES)

40 parts by weight of the active compound(s) are dissolved in 35 parts by weight of xylene with addition of calcium dodecylbenzenesulfonate and castor oil ethoxylate (in each case 5 parts by weight). This mixture is introduced into 30 parts by weight of water by means of an emulsifier machine (e.g. Ultraturrax) and made into a homogeneous emulsion. Dilution with water gives an emulsion, whereby a formulation with 25% (w/w) of active compound(s) is obtained.

### E) Suspensions (SC, OD, FS)

In an agitated ball mill, 20 parts by weight of the active compound(s) are comminuted with addition of 10 parts by weight of dispersants, wetters and 70 parts by weight of water or of an organic solvent to give a fine active compound(s) suspension. Dilution with water gives a stable suspension of the active compound(s), whereby a formulation with 20% (w/w) of active compound(s) is obtained.

### F) Water-Dispersible Granules and Water-Soluable Granules (WG, SG)

50 parts by weight of the active compound(s) are ground finely with addition of 50 parts by weight of dispersants and wetters and made as water-dispersible or water-soluble granules by means of technical appliances (for example extrusion, spray tower, fluidized bed). Dilution with water gives a stable dispersion or solution of the active compound(s), whereby a formulation with 50% (w/w) of active compound(s) is obtained.

### G) Water-Dispersible Powders and Water-Soluable Powders (WP, SP, SS, WS)

75 parts by weight of the active compound(s) are ground in a rotor-stator mill with addition of 25 parts by weight of dispersants, wetters and silica gel. Dilution with water gives a stable dispersion or solution of the active compound(s), whereby a formulation with 75% (w/w) of active compound(s) is obtained.

2. Products to be applied undiluted for foliar applications. For seed treatment purposes, such products may be applied to the seed diluted or undiluted.

### H) Dustable Powders (DP, DS)

5 parts by weight of the active compound(s) are ground finely and mixed intimately with 95 parts by weight of finely divided kaolin. This gives a dustable product having 5% (w/w) of active compound(s)

### I) Granules (GR, FG, GG, MG)

0.5 part by weight of the active compound(s) is ground finely and associated with 95.5 parts by weight of carriers, whereby a formulation with 0.5% (w/w) of active compound(s) is obtained. Current methods are extrusion, spray-drying or the fluidized bed. This gives granules to be applied undiluted for foliar use.

### J) ULV Solutions (UL, LS)

10 parts by weight of the active compound(s) are dissolved in 90 parts by weight of an organic solvent, for example xylene. This gives a product having 10% (w/w) of active compound(s), which is applied undiluted for foliar use.

Various types of oils, wetters, adjuvants, herbicides, fungicides, other pesticides, or bactericides may be added to the active ingredients, if appropriate just immediately prior to use (tank mix). These agents usually are admixed with the agents according to the invention in a weight ratio of 1:10 to 10:1.

The compounds of formula I or II are effective through both contact and ingestion.

The compounds of formula I or II are also suitable for the protection of the seed, plant propagules and the seedlings' roots and shoots, preferably the seeds, against soil pests and also for the treatment plant seeds which tolerate the action of herbicides or fungicides or insecticides owing to breeding, including genetic engineering methods.

Conventional seed treatment formulations include for example flowable concentrates FS, solutions LS, powders for dry treatment DS, water dispersible powders WS or granules for slurry treatment, water soluble powders SS and emulsion ES. Application to the seeds is carried out before sowing, either directly on the seeds.

The seed treatment application of the compounds of formula I or II or formulations containing them is carried out by spraying or dusting the seeds before sowing of the plants and before emergence of the plants.

The invention also relates to the propagation product of plants, and especially the treated seed comprising, that is, coated with and/or containing, a compound of formula I or II or a composition comprising it. The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

The seed comprises the inventive compounds or compositions comprising them in an amount of from 0.1 g to 10 kg per 100 kg of seed.

Compositions of this invention may also contain other active ingredients, for example other pesticides, insecticides, herbicides, fertilizers such as ammonium nitrate, urea, potash, and superphosphate, phytotoxicants and plant growth regulators, safeners and nematicides. These additional ingredients may be used sequentially or in combination with the above-described compositions, if appropriate also added only immediately prior to use (tank mix). For example, the plant(s) may be sprayed with a composition of this invention either before or after being treated with other active ingredients.

The following list of pesticides together with which the compounds according to the invention can be used, is intended to illustrate the possible combinations, but not to impose any limitation:

A.1. Organo(thio)phosphates: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, trichlorfon;
A.2. Carbamates: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;
A.3. Pyrethroids: allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, dimefluthrin;
A.4. Growth regulators: a) chitin synthesis inhibitors: benzoylureas: chlorfluazuron, diflubenzuron, flucycloxuron, flufenoxuron, hexaflumuron, lufenuron, novaluron, teflubenzuron, triflumuron; buprofezin, diofenolan, hexythiazox, etoxazole, clofentazine; b) ecdysone antagonists: halofenozide, methoxyfenozide, tebufenozide, azadirachtin; c) juvenoids: pyriproxyfen, methoprene, fenoxycarb; d) lipid biosynthesis inhibitors: spirodiclofen, spiromesifen, spirotetramat;
A.5. Nicotinic receptor agonists/antagonists compounds: clothianidin, dinotefuran, imidacloprid, thiamethoxam, nitenpyram, acetamiprid, thiacloprid; the thiazol compound of formula (Γ¹)

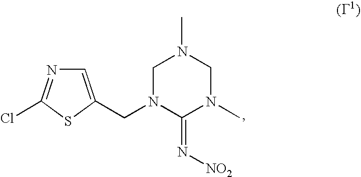

A.6. GABA antagonist compounds: acetoprole, endosulfan, ethiprole, fipronil, vaniliprole, pyrafluprole, pyriprole, the phenylpyrazole compound of formula Γ²

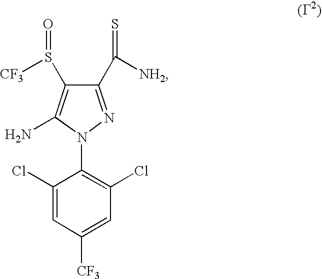

A.7. Macrocyclic lactone insecticides: abamectin, emamectin, milbemectin, lepimectin, spinosad;
A.8. METI I compounds: fenazaquin, pyridaben, tebufenpyrad, tolfenpyrad, flufenerim;
A.9. METI II and III compounds: acequinocyl, fluacyprim, hydramethylnon;
A. 10. Uncoupler compounds: chlorfenapyr;
A.11. Oxidative phosphorylation inhibitor compounds: cyhexatin, diafenthiuron, fenbutatin oxide, propargite;
A.12. Moulting disruptor compounds: cyromazine;
A.13. Mixed Function Oxidase inhibitor compounds: piperonyl butoxide;
A.14. Sodium channel blocker compounds: indoxacarb, metaflumizone,
A.15. Various: benclothiaz, bifenazate, cartap, flonicamid, pyridalyl, pymetrozine, sulfur, thiocyclam, flubendiamide, cyenopyrafen, flupyrazofos, cyflumetofen, amidoflumet, N—R′-2,2-dihalo-1-R′cyclo-propanecarboxamide-2-(2,6-dichloro-α,α,α-tri-fluoro-ptolyl)hydrazone or N—R′-2,2-di(R′″)propionamide-2-(2,6-dichloro-α,α,α-trifluoro-p-tolyl)hydrazone, wherein R′ is methyl or ethyl, halo is chloro or bromo, R″ is hydrogen or methyl and R′″ is methyl or ethyl, anthranilamide compounds of formula Γ³

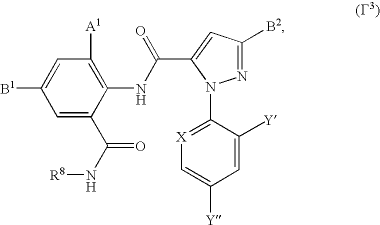

wherein A¹ is CH₃, Cl, Br, I, X is C—H, C—Cl, C—F or N, Y′ is F, Cl, or Br, Y″ is F, Cl, CF₃, B¹ is hydrogen, Cl, Br, I, CN, B² is Cl, Br, CF₃, OCH₂CF₃, OCF₂H, and R^{B} is hydrogen, CH₃ or CH(CH₃)₂, and malononitrile compounds as described in JP 2002 284608, WO 02/89579, WO 02/90320, WO 02/90321, WO 04/06677, WO 04/20399, or JP 2004 99597.

The commercially available compounds of the group A may be found in The Pesticide Manual, 13^{th} Edition, British Crop Protection Council (2003) among other publications. Thiamides of formula Γ² and their preparation have been described in WO 98/28279. Lepimection is known from Agro Project, PJB Publications Ltd, November 2004. Benclothiaz and its preparation have been described in EP-A1 454621. Methidathion and Paraoxon and their preparation have been described in Farm Chemicals Handbook, Volume 88, Meister Publishing Company, 2001. Acetoprole and its preparation have been described in WO 98/28277. Metaflumizone and its preparation have been described in EP-A1 462 456. Flupyrazofos has been described in Pesticide Science 54, 1988, p. 237-243 and in U.S. Pat. No. 4,822,779. Pyrafluprole and its preparation have been described in JP 2002193709 and in WO 01/00614. Pyriprole and its preparation have been described in WO 98/45274 and in U.S. Pat. No. 6,335,357. Amidoflumet and its preparation have been described in U.S. Pat. No. 6,221,890 and in JP 21010907. Flufenerim and its preparation have been described in WO 03/007717 and in WO 03/007718. Cyflumetofen and its preparation have been described in WO 04/080180.

The insects may be controlled by contacting the target parasite/pest, its food supply, habitat, breeding ground or its locus with a pesticidally effective amount of compounds of or compositions of formula I or II.

“Locus” means a habitat, breeding ground, plant, seed, soil, area, material or environment in which a pest or parasite is growing or may grow.

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

The compounds or compositions of the invention can also be applied preventively to locuses at which occurrence of the pests is expected.

The compounds of formula I or II may also be used to protect growing plants from attack or infestation by pests by contacting the plant with a pesticidally effective amount of compounds of formula I or II. As such, “contacting” includes both direct contact (applying the compounds/compositions directly on the pest and/or plant—typically to the foliage, stem or roots of the plant) and indirect contact (applying the compounds/compositions to the locus of the pest and/or plant).

In the case of soil treatment or of application to the pests dwelling place or nest, the quantity of active ingredient ranges from 0.0001 to 500 g per 100 m², preferably from 0.001 to 20 g per 100 m².

For use in treating crop plants, the rate of application of the active ingredients of this invention may be in the range of 0.1 g to 4000 g per hectare, desirably from 25 g to 600 g per hectare, more desirably from 50 g to 500 g per hectare.

### SYNTHESIS EXAMPLES

With due modification of the starting compounds, the protocols shown in the synthesis example below were used for obtaining further compounds I. The resulting compounds, together with physical data, are listed in the table 1.1 which follows.

The products were characterized by coupled High Performance Liquid Chromatography/mass spectrometry (HPLC/MS), by ¹H-NMR (400 MHz) in CDCl₃ or by their melting points. HPLC column: RP-18 column (Chromolith Speed ROD from Merck KgaA, Germany). Elution: acetonitrile+0.1% trifluoroacetic acid (TFA)/water in a ratio of from 5:95 to 95:5 in 5 minutes at 40° C. MS: Quadrupol electrospray ionisation, 80 V (positive modus). The m/z value given correlates to the molecule peak containing the lightest isotopes of all atoms.

### Example 1

### 3-(Azetidin-2-ylmethoxy)-pyridine-acetamid (* denotes the chiral centre)

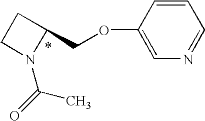

0.200 g (1.2 mmol) of 3-(azetidin-2-ylmethoxy)-pyridine were added to 20 ml of acetanhydride, and a catalytic amount of 4-dimethylaminopyridine was added. After stirring for 16 hours at 20-25° C., the solvent was removed in vacuo. Ethylacetate was added, and the mixture was washed with a 5% NaHCO₃ solution. The product was extracted with water. The water phase was treated with methylenechloride, and the solvent removed in vacuo. The crude product was subjected to column chromatography to afford 212 mg of the title compound as an oil. ¹H-NMR (CDCl₃): δ [ppm]=8.3-8.2 (m), 7.2 (m), 4.6 (m), 4.5 (m), 4.3-4.0 (m), 2.5 (m), 1.9 (m).

### Example 2

### [2-(5-Fluoro-pyridine-3-yloxymethyl)-azetidin-1-yl]-benzamide

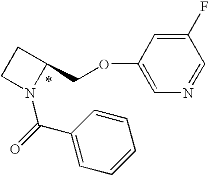

0.209 g (1.1 mmol) 3-(azetidin-2-ylmethoxy)-5-fluoro-pyridine were dissolved in a mixture of 7 ml tetrahydrofuran and 40 ml of acetonitrile, then 0.18 mg (1.3 mmol) of triethylamine and a catalytic amount of 4-dimethylaminopyridine and 0.170 g (1.2 mmol) of benzoylchloride in 5 ml tetrahydrofuran were added at 5° C. The mixture was stirred at 20-25° C. for 16 hours, the solvents were removed in vacuo, and the crude product subjected to column chromatography, yielding 236 mg of the above product (mp. 71-72° C.).

TABLE I.1

| | |
|---|---|
| | (I.1) |
| 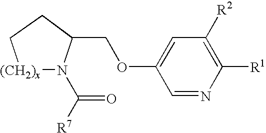 | |

| | | | | | Physical data |
|---|---|---|---|---|---|
| | | | | | ¹H-NMR (CDCl₃, δ [ppm]); HPLC/MS |
| No. | x | R¹ | R² | R⁷ | (RT [min]; m/z [M + H]⁺; mp. [° C.] |
| I.1-1 | 0 | H | Cl | C₆H₅ | 94° C. |
| I.1-2 | 0 | Cl | Cl | C₆H₅ | 96-97° C. |
| I.1-3 | 0 | Cl | H | C₆H₅ | 57-58° C. |
| I.1-4 | 0 | H | Cl | 4-CH₃—C₆H₄ | 103-104° C. |
| I.1-5 | 0 | H | Cl | cyclo-C₃H₅ | 80-81° C. |
| I.1-6 | 0 | H | Cl | 3-Cl—C₆H₄ | RT = 2.80 min, m/z = 336 [M + H]⁺ |
| I.1-7 | 0 | H | Cl | 3-CH₃—C₆H₄ | 88° C. |
| I.1-8 | 0 | H | Cl | 3-pyridyl | 100-101° C. |
| I.1-9 | 0 | H | Cl | 4-Cl—C₆H₄ | 93-94° C. |
| I.1-10 | 0 | H | Cl | 4-CF₃—C₆H₄ | 83-84° C. |
| I.1-11 | 0 | H | Cl | 3-CF₃—C₆H₄ | 73-75° C. |
| I.1-12 | 0 | H | Cl | 2-thiophenyl | 100° C. |
| I.1-13 | 0 | Cl | H | 3-CH₃—C₆H₄ | 65° C. |
| I.1-14 | 0 | Cl | H | 4-CF₃—C₆H₄ | 89-90° C. |
| I.1-15 | 0 | Cl | H | 3-thiophenyl | 80-81° C. |
| I.1-16 | 0 | Cl | Cl | 4-CH₃—C₆H₄ | 87-88° C. |
| I.1-17 | 0 | Cl | Cl | 4-NO₂—C₆H₄ | 77-78° C. |
| I.1-18 | 0 | Cl | Cl | 3-OCH₃—C₆H₄ | 66-67° C. |
| I.1-19 | 0 | Cl | Cl | 3-F—C₆H₄ | 45-46° C. |
| I.1-20 | 0 | Cl | Cl | 3-CF₃—C₆H₄ | 117-118° C. |
| I.1-21 | 0 | Cl | Cl | 3-thiophenyl | 102-103° C. |
| I.1-22 | 0 | H | Cl | 4-CH₃—C₆H₄ | 108-109° C. |
| I.1-23 | 0 | H | Cl | 4-Cl—C₆H₄ | RT = 2.88 min, m/z = 336.9 [M + H]⁺ |
| I.1-24 | 0 | H | Cl | 4-OCH₃—C₆H₄ | 96-97° C. |
| I.1-25 | 0 | H | Cl | 3-CF₃—C₆H₄ | 82-83° C. |
| I.1-26 | 0 | H | Cl | 4-F—C₆H₄ | RT = 2.59 min, m/z = 321 [M + H]⁺ |
| I.1-27 | 0 | H | Cl | 3-thiophenyl | RT = 2.42 min, m/z = 308.9 [M + H]⁺ |
| I.1-28 | 0 | Cl | H | 4-OCF₃—C₆H₄ | 63-64° C. |
| I.1-29 | 0 | Cl | H | 4-C₆H₅—C₆H₄ | 86-87° C. |
| I.1-30 | 0 | Cl | H | 3,4-(CH₃)₂—C₆H₃ | 73-74° C. |
| I.1-31 | 0 | Cl | H | 4-NC(═O)CH₃—C₆H₄ | 149-150° C. |
| I.1-32 | 0 | Cl | H | 4-SCH₃—C₆H₄ | 96-97° C. |
| I.1-33 | 0 | Cl | H | 3-tetrahydrofuranyl | RT = 2.12 min, m/z = 297 [M + H]⁺ |
| I.1-34 | 0 | Cl | H | 3-CH₃-isoxazol-5-yl | 81-82° C. |
| I.1-35 | 0 | Cl | H | 4-S(═O)₂CH₃—C₆H₄ | 117-118° C. |
| I.1-36 | 0 | Cl | H | 3-NC(═O)CH₃—C₆H₄ | 102-103° C. |
| I.1-37 | 0 | H | Br | C₆H₅ | RT = 2.60 min, m/z = 347 [M + H]⁺ |
| I.1-38 | 0 | Cl | H | 3-Cl,4-CH₃—C₆H₃ | 100-101° C. |
| I.1-39 | 0 | Cl | Cl | (CH₂)₂NH₂ | RT = 2.02 min, m/z = 304 [M + H]⁺ |
| I.1-40 | 0 | Cl | Cl | (CH₂)₃NH₂ | RT = 2.05 min, m/z = 318 [M + H]⁺ |
| I.1-41 | 0 | Cl | H | 3,5-(CH₃)₂—C₆H₃ | 83-84° C. |
| I.1-42 | 0 | Cl | H | 1,5-(CH₃)₂-pyrazol-3-yl | 104° C. |
| I.1-43 | 0 | H | CC—C(CH₃)₃ | C₆H₅ | RT = 2.16 min, m/z = 351 [M + H]⁺ |
| I.1-44 | 0 | Cl | Cl | 4-pyridyl | 84-86° C. |
| I.1-45 | 0 | Cl | F | C₆H₅ | 78-79° C. |
| I.1-46 | 0 | H | F | C₆H₅ | 71-72° C. |
| I.1-47 | 0 | Cl | H | 2-CF₃-pyrid-5-yl | 117-118° C. |
| I.1-48 | 0 | H | Br | 3-thiophenyl | 94-95° C. |
| I.1-49 | 0 | H | Br | cyclo-C₃H₅ | 86-87° C. |
| I.1-50 | 0 | Cl | H | 2-thiophenyl | RT = 2.70 min, m/z = 309 [M + H]⁺ |
| I.1-51 | 0 | Cl | H | 2-Cl-thiophen-5-yl | RT = 3.16 min, m/z = 343 [M + H]⁺ |
| I.1-52 | 0 | Cl | H | 3-furanyl | RT = 2.46 min, m/z = 293 [M + H]⁺ |
| I.1-53 | 0 | Cl | H | 4-Br—C₆H₄ | RT = 3.14 min, m/z = 381 [M + H]⁺ |
| I.1-54 | 0 | Cl | H | 4-C(═O)OCH₃—C₆H₄ | RT = 2.79 min, m/z = 361 [M + H]⁺ |
| I.1-55 | 0 | Cl | H | 3-CF₃-4-Cl—C₆H₃ | RT = 3.46 min, m/z = 405.2 [M + H]⁺ |
| I.1-56 | 0 | Cl | H | 3-Cl-4-OCH₃—C₆H₃ | RT = 3.01 min, m/z = 367.3 [M + H]⁺ |
| I.1-57 | 0 | Cl | H | 2-CH₃-pyrid-5-yl | RT = 1.76 min, m/z = 318.3 [M + H]⁺ |
| I.1-58 | 0 | Cl | H | 2-Cl-pyrid-5-yl | RT = 2.59 min, m/z = 338.3 [M + H]⁺ |
| I.1-59 | 0 | Cl | H | cyclo-C₅H₉ | RT = 2.85 min, m/z = 295.2 [M + H]⁺ |
| I.1-60 | 0 | Cl | H | 4-C(CH₃)₃—C₆H₃ | RT = 3.57 min, m/z = 359.4 [M + H]⁺ |
| I.1-61 | 0 | Cl | H | CH₂OCH₃ | RT = 2.06 min, m/z = 271.3 [M + H]⁺ |
| I.1-62 | 0 | Cl | H | 2-furanyl | RT = 2.50 min, m/z = 293.2 [M + H]⁺ |
| I.1-63 | 0 | Cl | H | C═CH₂ | RT = 2.21 min, m/z = 253.2 [M + H]⁺ |
| I.1-64 | 0 | H | Cl | 2-thiophenyl | RT = 2.55 min, m/z = 309.2 [M + H]⁺ |
| I.1-65 | 0 | H | Cl | 2-Cl-thiophen-5-yl | RT = 3.04 min, m/z = 343.1 [M + H]⁺ |
| I.1-66 | 0 | H | Cl | CH₂C₆H₅ | RT = 2.66 min, m/z = 317.3 [M + H]⁺ |
| I.1-67 | 0 | H | Cl | CCH | RT = 2.07 min, m/z = 251.3 [M + H]⁺ |
| I.1-68 | 1 | H | Cl | C₆H₅ | RT = 2.75 min, m/z = 317.2 [M = H]⁺ |
| I.1-69 | 1 | Cl | Cl | C₆H₅ | RT = 3.32 min, m/z = 351.0 [M + H]⁺ |
| I.1-70 | 1 | Cl | H | C₆H₅ | RT = 2.11 min, m/z = 317.2 [M + H]⁺ |
| I.1-71 | 0 | F | H | C₆H₅ | RT = 2.56 min, m/z = 287.1 [M + H]⁺ |
| I.1-72 | 0 | F | H | 3-tetrahydrofuranyl | RT = 2.10 min, m/z = 287.1 [M + H]⁺ |
| I.1-73 | 0 | F | H | cyclo-C₅H₉ | RT = 2.66 min, m/z = 279.1 [M + H]⁺ |
| I.1-74 | 0 | F | H | cyclo-C₆H₁₁ | RT = 2.86 min, m/z = 293.2 [M + H]⁺ |
| I.1-75 | 0 | F | H | 2-thiophenyl | RT = 2.53 min, m/z = 292.3 [M + H]⁺ |
| I.1-76 | 0 | H | CC-cyclo-C₅H₉ | C₆H₅ | RT = 3.07 min, m/z = 361.5 [M + H]⁺ |
| I.1-77 | 0 | H | CC—C₃H₇ | C₆H₅ | RT = 2.80 min, m/z = 335.4 [M + H]⁺ |

### Examples for the Action Against Harmful Pests

1. Activity Against Southern Armyworm (Spodoptera eridania), 2nd Instar Larvae

The active compounds are formulated for testing the activity against insects and arachnids as a 10.000 ppm solution in a mixture of 35% acetone and water, which is diluted with water, if needed.

A Sieva lima bean leaf is dipped in the test solution and allowed to dry. The leaf is then placed in a petri dish containing a filter paper on the bottom and ten 2nd instar caterpillars. At 5 days, observations are made of mortality and reduced feeding.

2. Activity Against Diamond Back Moth (Plutella xylostella)

The active compounds are formulated in 50:50 acetone:water and 0.1% (vol/vol) Alkamuls EL 620 surfactant. A 6 cm leaf disk of cabbage leaves is dipped in the test solution for 3 seconds and allowed to air dry in a Petri plate lined with moist filter paper. The leaf disk is inoculated with 10 third instar larvae and kept at 25-27° C. and 50-60% humidity for 3 days. Mortality is assessed after 72 h of treatment.

3. Activity Against Cowpea Aphid (Aphis craccivora)

The active compounds were formulated in 50:50 acetone:water. Potted cowpea plants colonized with 100-150 aphids of various stages were sprayed after the pest population has been recorded. Population reduction was recorded after 24, 72, and 120 hours.

In this test, compounds IA-923 of Table I, IA-923 of Table 3, IA-12 of Table 3, IA-1643 of Table I, IA-523 of Table I, IA-1003 of Table I, IA-1043 of Table I, IA-1603 of Table I, IA-1883 of Table I, IA-1483 of Table I, IA-1041 of Table 3, IA-1043 of Table 3, IA-1001 of Table 3, IA-1601 of Table 3, IA-1801 of Table 3, IA-1281 of Table 3, IA-761 of Table 3, IA-721 of Table 3, IA-1643 of Table 3, IA-521 of Table 3, IA-161 of Table 3, IA-81 of Table 3, IA-4 of Table I, IA-2001 of Table 3, I.1-72, I.1-73, I.1-74, I.1-75, I.1-76, I.1-77, and I.1-78, at 300 ppm showed over 90% mortality compared to 0% mortality of untreated controls.

5. Activity Against Vetch Aphid (Megoura viciae)

The active compounds are formulated in 1:3 DMSO:water. Bean leaf disks are placed into microtiterplates filled with 0.8% agar-agar and 2.5 ppm OPUS™. The leaf disks are sprayed with 2.5 μl of the test solution and 5 to 8 adult aphids are placed into the microtiterplates which are then closed and kept at 22-24° C. and 35-45% under fluorescent light for 6 days. Mortality is assessed on the basis of vital, reproduced aphids. Tests are replicated 2 times.

6. Activity Against Wheat Aphid (Rhopalosiphum padi)

The active compounds are formulated in 1:3 DMSO:water. Barlay leaf disk are placed into microtiterplates filled with 0.8% agar-agar and 2.5 ppm OPUS™. The leaf disks are sprayed with 2.5 μl of the test solution and 3 to 8 adult aphids are placed into the microtiterplates which are then closed and kept at 22-24° C. and 35-45% humidity under fluorescent light for 5 days. Mortality is assessed on the basis of vital aphids. Tests are replicated 2 times.

7. Activity Against Cotton Aphid (Aphis gossypii)

The active compounds were formulated in 50:50 acetone:water and 100 ppm Kinetic™ surfactant.

Cotton plants at the cotyledon stage (one plant per pot) were infested by placing a heavily infested leaf from the main colony on top of each cotyledon. The aphids were allowed to transfer to the host plant overnight, and the leaf used to transfer the aphids was removed. The cotyledons were dipped in the test solution and allowed to dry. After 5 days, mortality counts were made.

In this test, compounds IA-923 of Table I, IA-923 of Table 3, IA-12 of Table 3, IA-1643 of Table I, IA-523 of Table I, IA-1003 of Table I, IA-1043 of Table I, IA-1603 of Table I, IA-1883 of Table I, IA-1483 of Table I, IA-1041 of Table 3, IA-1043 of Table 3, IA-1001 of Table 3, IA-1601 of Table 3, IA-1801 of Table 3, IA-1281 of Table 3, IA-761 of Table 3, IA-721 of Table 3, IA-1643 of Table 3, IA-521 of Table 3, IA-161 of Table 3, IA-81 of Table 3, IA-4 of Table I, IA-2001 of Table 3, I.1-72, I.1-73, I.1-74, I.1-75, I.1-76, I.1-77, and I.1-78, at 300 ppm showed over 90% mortality compared to 0% mortality of untreated controls.

8. Activity Against Silverleaf Whitefly (Bemisia argentifolii)

The active compounds were formulated in 50:50 acetone:water and 100 ppm Kinetic™ surfactant.

Selected cotton plants were grown to the cotyledon state (one plant per pot). The cotyledons were dipped into the test solution to provide complete coverage of the foliage and placed in a well-vented area to dry. Each pot with treated seedling was placed in a plastic cup and 10 to 12 whitefly adults (approximately 3-5 day old) were introduced. The insects were collected using an aspirator and an 0.6 cm, non-toxic Tygon™ tubing (R-3603) connected to a barrier pipette tip. The tip, containing the collected insects, was then gently inserted into the soil containing the treated plant, allowing insects to crawl out of the tip to reach the foliage for feeding. The cups were covered with a reusable screened lid (150 micron mesh polyester screen PeCap from Tetko Inc). Test plants were maintained in the holding room at about 25° C. and 20-40% relative humidity for 3 days avoiding direct exposure to the fluorescent light (24 hour photoperiod) to prevent trapping of heat inside the cup. Mortality was assessed 3 days after treatment of the plants.

In this test, compounds IA-1641 of Table 3 IA-523 of Table 1, I.1-73, I.1-74, I.1-76, and I.1-78 at 300 ppm showed over 75% mortality compared to 0% mortality of untreated controls.

9. Green Peach Aphid (Myzus persicae)

The active compounds were formulated in 50:50 acetone:water and 100 ppm Kinetic™ surfactant.

Pepper plants in the 2^{nd} leaf-pair stage (variety ‘California Wonder’) were infested with approximately 40 laboratory-reared aphids by placing infested leaf sections on top of the test plants. The leaf sections were removed after 24 hr. The leaves of the intact plants were dipped into gradient solutions of the test compound and allowed to dry. Test plants were maintained under fluorescent light (24 hour photoperiod) at about 25° C. and 20-40% relative humidity. Aphid mortality on the treated plants, relative to mortality on check plants, was determined after 5 days.

In this test, compounds IA-923 of Table I, IA-923 of Table 3, IA-12 of Table 3, IA-1643 of Table I, IA-523 of Table I, IA-1003 of Table I, IA-1043 of Table I, IA-1603 of Table I, IA-1883 of Table I, IA-1483 of Table I, IA-1041 of Table 3, IA-1043 of Table 3, IA-1001 of Table 3, IA-1601 of Table 3, IA-1801 of Table 3, IA-1281 of Table 3, IA-761 of Table 3, IA-721 of Table 3, IA-1643 of Table 3, IA-521 of Table 3, IA-161 of Table 3, IA-81 of Table 3, IA-4 of Table I, IA-2001 of Table 3, I.1-72, I.1-73, I.1-74, I.1-75, I.1-76, I.1-77, and I.1-78 at 300 ppm showed over 90% mortality compared to 0% mortality of untreated controls.

1-15. (canceled)

16: A method of combating pests or for protecting growing plants from attack or infestation by pests, comprising the step of applying to a pest or its food supply, habitat, breeding ground or locus, or to a plant or to the soil or the water in which the plant is growing a pesticidally effective amount of a 3-pyridyl derivative of formula I 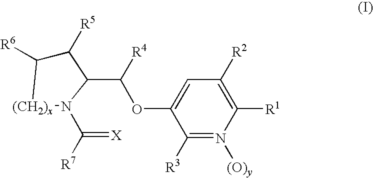
wherein
X is oxygen or sulfur;
R¹ and R² are each independently hydrogen, halogen, cyano, nitro, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₆-halocycloalkyl, C₃-C₆-cycloalkenyl, C₃-C₆-halocycloalkenyl, C₁-C₆-alkyl-C₃-C₆-cycloalkyl, ORⁱ, SRⁱ, S(═O)Rⁱ, S(═O)₂Rⁱ, NRⁱR^{j}, C(═O)ORⁱ, SiRⁱ_{z}R^{j}_{3-z} (z is 0 to 3), or
phenyl or a 5- to 6-membered heteroaromatic ring which may contain 1 to 4 heteroatoms selected from the group consisting of oxygen, nitrogen and sulfur, or a 3- to 7-membered saturated or partially unsaturated heterocyclic ring which may contain 1 to 3 heteroatoms selected from the group consisting of oxygen, nitrogen and sulfur,
wherein the carbon atoms in phenyl or in the heteroaromatic or heterocyclic ring may be substituted with 1 to 3 groups selected from the group consisting of halogen, amino, cyano, Rⁱ, ORⁱ, SRⁱ and nitro;
Rⁱ, R^{j} are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₈-halocycloalkyl, C₃-C₆-cycloalkenyl, C₃-C₆-halocycloalkenyl, C₁-C₆-haloalkoxy, C₁-C₄-alkoxy-C₁-C₆-alkyl, C₁-C₄-alkylthio-C₁-C₆-alkyl, C₁-C₄-alkylsulfinyl-C₁-C₆-alkyl, C₁-C₄-alkylsulfonyl-C₁-C₆-alkyl, C₁-C₄-haloalkoxy-C₁-C₆-alkyl, C₁-C₄-haloalkylthio-C₁-C₆-alkyl, (C₁-C₄-alkoxy)carbonyl-C₁-C₆-alkyl, (C₁-C₄-alkyl)amino-C₁-C₆-alkyl, di(C₁-C₄-alkyl)amino-C₁-C₆-alkyl, C₃-C₆-cycloalkyl-C₁-C₆-alkyl, phenyl-C₁-C₆-alkyl, or C₁-C₆-alkyl which is substituted with 1 to 3 cyano groups;
R³ is hydrogen, halogen or C₁-C₆-alkyl;
R⁴ is hydrogen or C₁-C₆-alkyl;
R⁵ and R⁶ are each independently hydrogen, halogen, cyano, or C₁-C₆-alkyl;
R⁷ is C₁-C₆-alkyl, C₂-C₆-alkenyl, C₂-C₆-alkynyl, C₃-C₆-cycloalkyl, C₃-C₆-cycloalkenyl, C₁-C₆-haloalkyl, C₂-C₆-haloalkenyl, C₂-C₆-haloalkynyl, C₃-C₆-halocycloalkyl, C₃-C₆-halocycloalkenyl, —C(=G)R^{a}, —C(=G)OR^{a}, —C(=G)NR^{a}R^{b}, —C(═NOR^{a})R^{b}, or C₃-C₆-cycloalkyl-C₁-C₆-alkyl; wherein
G is oxygen or sulfur;
R^{a}, R^{b} are each independently halogen, cyano, nitro, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₆-halocycloalkyl, C₃-C₆-cycloalkenyl, C₃-C₆-halocycloalkenyl, OR^{k}, SR^{k}, S(═O)R^{k}, S(═O)₂R^{k}, S(═O)₂NR^{k}R^{m}, C(═O)R^{k}, C(═O)OR^{k}, C(═O)NR^{k}R^{m}, C(═NOR^{k})R^{m}, —C(=G)NR^{k}—NR^{m}Rⁿ, SiR^{k}_{z}R^{m}_{3-z} wherein z is 0 to 3, or
R^{a}, R^{b} are each independently phenyl or a 5- to 6-membered heteroaromatic ring which may contain 1 to 4 heteroatoms selected from the group consisting of oxygen, nitrogen and sulfur, or a 4- to 7-membered saturated or partially unsaturated heterocyclic ring which may contain 1 to 3 heteroatoms selected from the group consisting of oxygen, nitrogen and sulfur, wherein the carbon atoms in phenyl or in the heteroaromatic or heterocyclic ring may be substituted with 1 to 5 halogens;
R^{k}, R^{m}, Rⁿ are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₈-halocycloalkyl, C₃-C₆-cycloalkenyl, or C₃-C₆-halocycloalkenyl; or
R⁷ is phenyl or a 3- to 7-membered saturated or partially unsaturated heterocyclic ring which may contain 1 to 3 heteroatoms selected from the group consisting of oxygen, sulfur and nitrogen or a 5- to 6-membered heteroaromatic ring system which may contain 1 to 4 heteroatoms selected from the group consisting of oxygen, nitrogen and sulfur, which phenyl, heterocyclic ring or heteroaromatic ring is bonded via a carbon atom of the ring or which phenyl, heterocyclic ring or heteroaromatic ring may be bonded via C₁-C₄-alkyl,
wherein phenyl or the heteroaromatic ring or the heterocyclic ring may be fused to a ring selected from the group consisting of phenyl and a 5- to 6-membered saturated, partially unsaturated or aromatic heterocyclic ring which may contain 1 to 3 heteroatoms selected from the group consisting of oxygen, nitrogen and sulfur,
wherein the above groups R⁷ are unsubstituted, or the hydrogen atoms in these groups may all or in part be replaced with any combination of groups selected from R^{c};
R^{c} is each independently halogen, cyano, nitro, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₈-halocycloalkyl, C₃-C₆-cycloalkenyl, C₃-C₈-halocycloalkenyl, OR^{k}, SR^{k}, S(═O)R^{k}, S(═O)₂R^{k}, NR^{k}R^{m}, N(OR^{k})R^{m}, —S(═O)₂NR^{k}R^{m}, C(═O)R^{k}, C(═O)OR^{k}, C(═O)NR^{k}R^{m}, C(═NOR^{k})R^{m}, —NR^{k}C(=G)R^{m}, —N(OR^{k})C(=G)R^{m}, —N[C(=G)R^{k}][C(=G)R^{m}], —NR^{k}C(=G)OR^{m}, —N(OR^{k})C(=G)OR^{m}, —C(=G)NR^{k}NR^{m}Rⁿ, —NR^{k}SO₂R^{m}, SiR^{k}R^{m}_{3-z} wherein z is 0 to 3, or
R^{c} is each independently phenyl or a 5- to 6-membered heteroaromatic ring which may contain 1 to 4 heteroatoms selected from the group consisting of oxygen, nitrogen and sulfur, or a 4- to 7-membered saturated or partially unsaturated heterocyclic ring which may contain 1 to 3 heteroatoms selected from the group consisting of oxygen, nitrogen and sulfur, wherein the carbon atoms in phenyl or in the heteroaromatic or heterocyclic ring may be substituted with 1 to 5 halogens; wherein
R^{k}, R^{m}, Rⁿ are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₈-halocycloalkyl, C₃-C₆-cycloalkenyl, C₃-C₆-halocycloalkenyl;
x is 0 or 1; and
y is 0 or 1;
or a diastereomer, enantiomer or salt thereof, or
a 3-pyridyl derivative of formula II 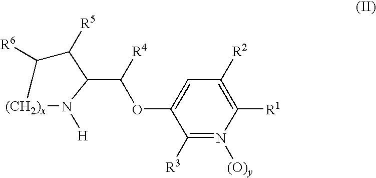
wherein
x, y are zero;
R⁴ is hydrogen or C₁-C₄-alkyl;
R¹, R² are each independently hydrogen, halogen, cyano, C₁-C₄-alkyl, C₁-C₄-haloalkyl, C₁-C₄-alkoxy, C₁-C₄-alkylthio, C₁-C₄-alkylsulfinyl, C₁-C₄-alkylsulfonyl, C₁-C₄-haloalkylthio, C₁-C₄-haloalkylsulfinyl, C₁-C₄-haloalkylsulfonyl; and
R³, R⁵, R⁶ are hydrogen
or a diastereomer, enantiomer or salt thereof; or
a composition, comprising
(a) a compound of formula (I), a compound of formula (II) or combinations thereof and
(b) an agronomically acceptable carrier.

17: The method according to claim 16, wherein the composition is formulated into (a) a dusting powder or granules, (b) a dispersible powder, granules or grains, or (c) an aqueous dispersion, suspension, paste, or emulsion.

18: The method according to claim 16, wherein the pesticidally effective amount of the compound of formula I or II or the composition comprising the compound of formula I or II is contacted with the pest or its food supply, habitat, breeding ground or locus.

19: The method according to claim 16, wherein the pesticidally effective amount of the compound of formula I or II or the composition comprising the compound of formula I or II is contacted with the plant, or to the soil or the water in which the plant is growing.

20: The method according to claim 16, wherein the composition or the compound of formula I or II is applied at a rate of 5 g/ha to 2000 g/ha.

21: The method according to claim 16, wherein the pest is an insect.

22: A method of protection of seed comprising contacting the seed with a pesticidally effective amount of a 3-pyridyl derivative of formula I 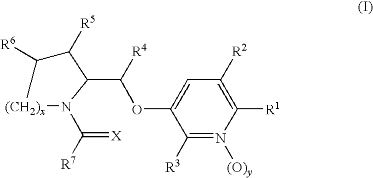
wherein
X is oxygen or sulfur;
R¹ and R² are each independently hydrogen, halogen, cyano, nitro, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₆-halocycloalkyl, C₃-C₆-cycloalkenyl, C₃-C₆-halocycloalkenyl, C₁-C₆-alkyl-C₃-C₆-cycloalkyl, ORⁱ, SRⁱ, S(═O)Rⁱ, S(═O)₂Rⁱ, NRⁱR^{j}, C(═O)ORⁱ, SiRⁱ_{z}R^{j}_{3-z} (z is 0 to 3), or
phenyl or a 5- to 6-membered heteroaromatic ring which may contain 1 to 4 heteroatoms selected from the group consisting of oxygen, nitrogen and sulfur, or a 3- to 7-membered saturated or partially unsaturated heterocyclic ring which may contain 1 to 3 heteroatoms selected from the group consisting of oxygen, nitrogen and sulfur,
wherein the carbon atoms in phenyl or in the heteroaromatic or heterocyclic ring may be substituted with 1 to 3 groups selected from the group consisting of halogen, amino, cyano, Rⁱ, ORⁱ, SRⁱ and nitro;
Rⁱ, R^{j} are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₈-halocycloalkyl, C₃-C₆-cycloalkenyl, C₃-C₆-halocycloalkenyl, C₁-C₆-haloalkoxy, C₁-C₄-alkoxy-C₁-C₆-alkyl, C₁-C₄-alkylthio-C₁-C₆-alkyl, C₁-C₄-alkylsulfinyl-C₁-C₆-alkyl, C₁-C₄-alkylsulfonyl-C₁-C₆-alkyl, C₁-C₄-haloalkoxy-C₁-C₆-alkyl, C₁-C₄-haloalkylthio-C₁-C₆-alkyl, (C₁-C₄-alkoxy)carbonyl-C₁-C₆-alkyl, (C₁-C₄-alkyl)amino-C₁-C₆-alkyl, di(C₁-C₄-alkyl)amino-C₁-C₆-alkyl, C₃-C₆-cycloalkyl-C₁-C₆-alkyl, phenyl-C₁-C₆-alkyl, or C₁-C₆-alkyl which is substituted with 1 to 3 cyano groups;
R³ is hydrogen, halogen or C₁-C₆-alkyl;
R⁴ is hydrogen or C₁-C₆-alkyl;
R⁵ and R⁶ are each independently hydrogen, halogen, cyano, or C₁-C₆-alkyl;
R⁷ is C₁-C₆-alkyl, C₂-C₆-alkenyl, C₂-C₆-alkynyl, C₃-C₆-cycloalkyl, C₃-C₆-cycloalkenyl, C₁-C₆-haloalkyl, C₂-C₆-haloalkenyl, C₂-C₆-haloalkynyl, C₃-C₆-halocycloalkyl, C₃-C₆-halocycloalkenyl, —C(=G)R^{a}, —C(=G)OR^{a}, —C(=G)NR^{a}R^{b}, —C(═NOR^{a})R^{b}, or C₃-C₆-cycloalkyl-C₁-C₆-alkyl; wherein
G is oxygen or sulfur;
R^{a}, R^{b} are each independently halogen, cyano, nitro, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₆-halocycloalkyl, C₃-C₆-cycloalkenyl, C₃-C₆-halocycloalkenyl, OR^{k}, SR^{k}, S(═O)R^{k}, S(═O)₂R^{k}, S(═O)₂NR^{k}R^{m}, C(═O)R^{k}, C(═O)OR^{k}, C(═O)NR^{k}R^{m}, C(═NOR^{k})R^{m}, —C(=G)NR^{k}—NR^{m}Rⁿ, SiR^{k}_{z}R^{m}_{3-z} wherein z is 0 to 3, or
R^{a}, R^{b} are each independently phenyl or a 5- to 6-membered heteroaromatic ring which may contain 1 to 4 heteroatoms selected from the group consisting of oxygen, nitrogen and sulfur, or a 4- to 7-membered saturated or partially unsaturated heterocyclic ring which may contain 1 to 3 heteroatoms selected from the group consisting of oxygen, nitrogen and sulfur, wherein the carbon atoms in phenyl or in the heteroaromatic or heterocyclic ring may be substituted with 1 to 5 halogens;
R^{k}, R^{m}, Rⁿ are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₈-halocycloalkyl, C₃-C₆-cycloalkenyl, or C₃-C₆-halocycloalkenyl; or
R⁷ is phenyl or a 3- to 7-membered saturated or partially unsaturated heterocyclic ring which may contain 1 to 3 heteroatoms selected from the group consisting of oxygen, sulfur and nitrogen or a 5- to 6-membered heteroaromatic ring system which may contain 1 to 4 heteroatoms selected from the group consisting of oxygen, nitrogen and sulfur, which phenyl, heterocyclic ring or heteroaromatic ring is bonded via a carbon atom of the ring or which phenyl, heterocyclic ring or heteroaromatic ring may be bonded via C₁-C₄-alkyl,
wherein phenyl or the heteroaromatic ring or the heterocyclic ring may be fused to a ring selected from the group consisting of phenyl and a 5- to 6-membered saturated, partially unsaturated or aromatic heterocyclic ring which may contain 1 to 3 heteroatoms selected from the group consisting of oxygen, nitrogen and sulfur,
wherein the above groups R⁷ are unsubstituted, or the hydrogen atoms in these groups may all or in part be replaced with any combination of groups selected from R^{c};
R^{c} is each independently halogen, cyano, nitro, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₈-halocycloalkyl, C₃-C₆-cycloalkenyl, C₃-C₈-halocycloalkenyl, OR^{k}, SR^{k}, S(═O)R^{k}, S(═O)₂R^{k}, NR^{k}R^{m}, N(OR^{k})R^{m}, —S(═O)₂NR^{k}R^{m}, C(═O)R^{k}, C(═O)OR^{k}, C(═O)NR^{k}R^{m}, C(═NOR^{k})R^{m}, —NR^{k}C(=G)R^{m}, —N(OR^{k})C(=G)R^{m}, —N[C(=G)R^{k}][C(=G)R^{m}], —NR^{k}C(=G)OR^{m}, —N(OR^{k})C(=G)OR^{m}, —C(=G)NR^{k}—NR^{m}Rⁿ, —NR^{k}SO₂R^{m}, SiR^{k}_{z}R^{m}_{3-z} wherein z is 0 to 3, or
R^{c} is each independently phenyl or a 5- to 6-membered heteroaromatic ring which may contain 1 to 4 heteroatoms selected from the group consisting of oxygen, nitrogen and sulfur, or a 4- to 7-membered saturated or partially unsaturated heterocyclic ring which may contain 1 to 3 heteroatoms selected from the group consisting of oxygen, nitrogen and sulfur, wherein the carbon atoms in phenyl or in the heteroaromatic or heterocyclic ring may be substituted with 1 to 5 halogens; wherein
R^{k}, R^{m}, Rⁿ are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₈-halocycloalkyl, C₃-C₆-cycloalkenyl, C₃-C₆-halocycloalkenyl;
x is 0 or 1; and
y is 0 or 1;
or a diastereomer, enantiomer or salt thereof, or
a 3-pyridyl derivative of formula II 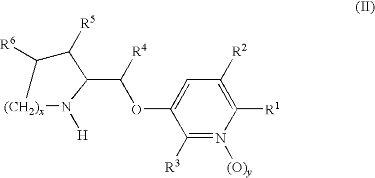
wherein
x, y are zero;
R⁴ is hydrogen or C₁-C₄-alkyl;
R¹, R² are each independently hydrogen, halogen, cyano, C₁-C₄-alkyl, C₁-C₄-haloalkyl, C₁-C₄-alkoxy, C₁-C₄-alkylthio, C₁-C₄-alkylsulfinyl, C₁-C₄-alkylsulfonyl, C₁-C₄-haloalkylthio, C₁-C₄-haloalkylsulfinyl, C₁-C₄-haloalkylsulfonyl; and
R³, R⁵, R⁶ are hydrogen
or a diastereomer, enantiomer or salt thereof; or
a composition, comprising
(a) a compound of formula (I), a compound of formula (II) or combinations thereof and
(b) an agronomically acceptable carrier.

23: The method as claimed in claim 22, wherein the composition or compound of formula I or II is applied in an amount of from 0.1 g to 10 kg per 100 kg of seeds.

24: A seed, comprising an amount of from 0.1 g to 10 kg per 100 kg of seed of a 3-pyridyl derivative of formula I 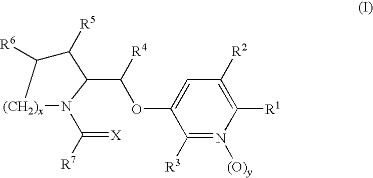
wherein
X is oxygen or sulfur;
R¹ and R² are each independently hydrogen, halogen, cyano, nitro, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₆-halocycloalkyl, C₃-C₆-cycloalkenyl, C₃-C₆-halocycloalkenyl, C₁-C₆-alkyl-C₃-C₆-cycloalkyl, ORⁱ, SRⁱ, S(═O)Rⁱ, S(═O)₂Rⁱ, NRⁱR^{j}, C(═O)ORⁱ, SiRⁱ_{z}R^{j}_{3-z} (z is 0 to 3), or
phenyl or a 5- to 6-membered heteroaromatic ring which may contain 1 to 4 heteroatoms selected from the group consisting of oxygen, nitrogen and sulfur, or a 3- to 7-membered saturated or partially unsaturated heterocyclic ring which may contain 1 to 3 heteroatoms selected from the group consisting of oxygen, nitrogen and sulfur,
wherein the carbon atoms in phenyl or in the heteroaromatic or heterocyclic ring may be substituted with 1 to 3 groups selected from the group consisting of halogen, amino, cyano, Rⁱ, ORⁱ, SRⁱ and nitro;
Rⁱ, R^{j} are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₈-halocycloalkyl, C₃-C₆-cycloalkenyl, C₃-C₆-halocycloalkenyl, C₁-C₆-haloalkoxy, C₁-C₄-alkoxy-C₁-C₆-alkyl, C₁-C₄-alkylthio-C₁-C₆-alkyl, C₁-C₄-alkylsulfinyl-C₁-C₆-alkyl, C₁-C₄-alkylsulfonyl-C₁-C₆-alkyl, C₁-C₄-haloalkoxy-C₁-C₆-alkyl, C₁-C₄-haloalkylthio-C₁-C₆-alkyl, (C₁-C₄-alkoxy)carbonyl-C₁-C₆-alkyl, (C₁-C₄-alkyl)amino-C₁-C₆-alkyl, di(C₁-C₄-alkyl)amino-C₁-C₆-alkyl, C₃-C₆-cycloalkyl-C₁-C₆-alkyl, phenyl-C₁-C₆-alkyl, or C₁-C₆-alkyl which is substituted with 1 to 3 cyano groups;
R³ is hydrogen, halogen or C₁-C₆-alkyl;
R⁴ is hydrogen or C₁-C₆-alkyl;
R⁵ and R⁶ are each independently hydrogen, halogen, cyano, or C₁-C₆-alkyl;
R⁷ is C₁-C₆-alkyl, C₂-C₆-alkenyl, C₂-C₆-alkynyl, C₃-C₆-cycloalkyl, C₃-C₆-cycloalkenyl, C₁-C₆-haloalkyl, C₂-C₆-haloalkenyl, C₂-C₆-haloalkynyl, C₃-C₆-halocycloalkyl, C₃-C₆-halocycloalkenyl, —C(=G)R^{a}, —C(=G)OR^{a}, —C(=G)NR^{a}R^{b}, —C(═NOR^{a})R^{b}, or C₃-C₆-cycloalkyl-C₁-C₆-alkyl; wherein
G is oxygen or sulfur;
R^{a}, R^{b} are each independently halogen, cyano, nitro, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₆-halocycloalkyl, C₃-C₆-cycloalkenyl, C₃-C₆-halocycloalkenyl, OR^{k}, SR^{k}, S(═O)R^{k}, S(═O)₂R^{k},S(═O)₂NR^{k}R^{m}, C(═O)R^{k}, C(═O)OR^{k}, C(═O)NR^{k}R^{m}, C(═NOR^{k})R^{m}, —C(=G)NR^{k}—NR^{m}Rⁿ, SiR^{k}_{z}R^{m}_{3-z} wherein z is 0 to 3, or
R^{a}, R^{b} are each independently phenyl or a 5- to 6-membered heteroaromatic ring which may contain 1 to 4 heteroatoms selected from the group consisting of oxygen, nitrogen and sulfur, or a 4- to 7-membered saturated or partially unsaturated heterocyclic ring which may contain 1 to 3 heteroatoms selected from the group consisting of oxygen, nitrogen and sulfur, wherein the carbon atoms in phenyl or in the heteroaromatic or heterocyclic ring may be substituted with 1 to 5 halogens;
R^{k}, R^{m}, Rⁿ are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₈-halocycloalkyl, C₃-C₆-cycloalkenyl, or C₃-C₆-halocycloalkenyl; or
R⁷ is phenyl or a 3- to 7-membered saturated or partially unsaturated heterocyclic ring which may contain 1 to 3 heteroatoms selected from the group consisting of oxygen, sulfur and nitrogen or a 5- to 6-membered heteroaromatic ring system which may contain 1 to 4 heteroatoms selected from the group consisting of oxygen, nitrogen and sulfur, which phenyl, heterocyclic ring or heteroaromatic ring is bonded via a carbon atom of the ring or which phenyl, heterocyclic ring or heteroaromatic ring may be bonded via C₁-C₄-alkyl,
wherein phenyl or the heteroaromatic ring or the heterocyclic ring may be fused to a ring selected from the group consisting of phenyl and a 5- to 6-membered saturated, partially unsaturated or aromatic heterocyclic ring which may contain 1 to 3 heteroatoms selected from the group consisting of oxygen, nitrogen and sulfur,
wherein the above groups R⁷ are unsubstituted, or the hydrogen atoms in these groups may all or in part be replaced with any combination of groups selected from R^{c};
R^{c} is each independently halogen, cyano, nitro, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₈-halocycloalkyl, C₃-C₆-cycloalkenyl, C₃-C₈-halocycloalkenyl, OR^{k}, SR^{k}, S(═O)R^{k}, S(═O)₂R^{k}, NR^{k}R^{m}, N(OR^{k})R^{m}, —S(═O)₂NR^{k}R^{m}, C(═O)R^{k}, C(═O)OR^{k}, C(═O)NR^{k}R^{m}, C(═NOR^{k})R^{m}, —NR^{k}C(=G)R^{m}, —N(OR^{k})C(=G)R^{m}, —N[C(=G)R^{k}][C(=G)R^{m}], —NR^{k}C(=G)OR^{m}, —N(OR^{k})C(=G)OR^{m}, —C(=G)NR^{k}NR^{m}Rⁿ, —NR^{k}SO₂R^{m}, SiR^{k}_{z}R^{m}_{3-z} wherein z is 0 to 3, or
R^{c} is each independently phenyl or a 5- to 6-membered heteroaromatic ring which may contain 1 to 4 heteroatoms selected from the group consisting of oxygen, nitrogen and sulfur, or a 4- to 7-membered saturated or partially unsaturated heterocyclic ring which may contain 1 to 3 heteroatoms selected from the group consisting of oxygen, nitrogen and sulfur, wherein the carbon atoms in phenyl or in the heteroaromatic or heterocyclic ring may be substituted with 1 to 5 halogens; wherein
R^{k}, R^{m}, Rⁿ are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₈-halocycloalkyl, C₃-C₆-cycloalkenyl, C₃-C₆-halocycloalkenyl;
x is 0 or 1; and
y is 0 or 1;
or a diastereomer, enantiomer or salt thereof, or
a 3-pyridyl derivative of formula II 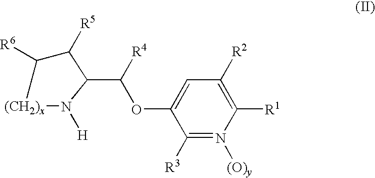
wherein
x, y are zero;
R⁴ is hydrogen or C₁-C₄-alkyl;
R¹, R² are each independently hydrogen, halogen, cyano, C₁-C₄-alkyl, C₁-C₄-haloalkyl, C₁-C₄-alkoxy, C₁-C₄-alkylthio, C₁-C₄-alkylsulfinyl, C₁-C₄-alkylsulfonyl, C₁-C₄-haloalkylthio, C₁-C₄-haloalkylsulfinyl, C₁-C₄-haloalkylsulfonyl; and
R³, R⁵, R⁶ are hydrogen
or a diastereomer, enantiomer or salt thereof.

25: A compound of formula I 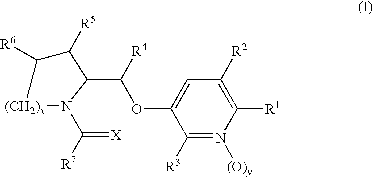
wherein
R¹ is fluoro, R², R³, R⁴, R⁵ and R⁶ are hydrogen, x and y are zero, X is oxygen, and R⁷ is selected from the group consisting of 2-oxo-tetrahydrofuran-4-yl, phenyl which may be substituted in the 4-position with nitro, fluoro, chloro, methyl, carboxymethyl, methoxy, or diethylaminomethyl and phenyl which is substituted in the 2-position with hydroxymethyl,
with the proviso that that compounds where x and y are zero, X is oxygen, R⁴, R⁵ and R⁶ are hydrogen and R⁷ is straight or branched C₁-C₆-alkyl are excluded.

26: A process for the preparation of a compound (1-1) wherein
R¹ is fluoro, R², R³, R⁴, R⁵ and R⁶ are hydrogen, x and y are zero, X is oxygen, and R⁷ is selected from the group consisting of 2-oxo-tetrahydrofuran-4-yl, phenyl which may be substituted in the 4-position with nitro, fluoro, chloro, methyl, carboxymethyl, methoxy, or diethylaminomethyl and phenyl which is substituted in the 2-position with hydroxymethyl,
with the proviso that that compounds where x and y are zero, X is oxygen, R⁴, R⁵ and R⁶ are hydrogen and R⁷ is straight or branched C₁-C₆-alkyl are excluded;
comprising reacting an amine of formula (II) 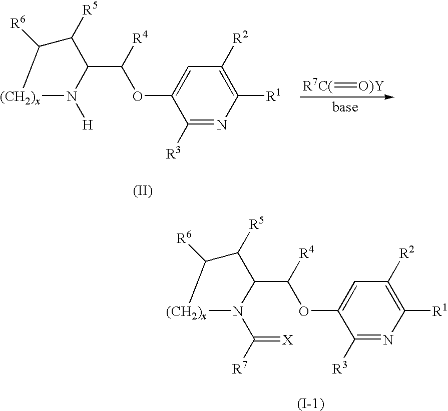
with an activated carboxylic acid derivative R⁷C(═O)Y in the presence of a base, wherein Y is OH or a suitable leaving group, chlorine, bromine, OR^{d}, OC(═O)R^{e}, or imidazole, wherein R^{d} is C₁-C₆-alkyl or N-hydroxybenzotriazole and R^{e} is C₁-C₆-alkyl or phenyl.

27: A pesticidal composition, comprising
(a) a compound of the formula (I) of claim 25
(b) an agronomically acceptable carrier.

28: A synergistic pesticidal mixture, comprising
a 3-pyridyl derivative of formula I 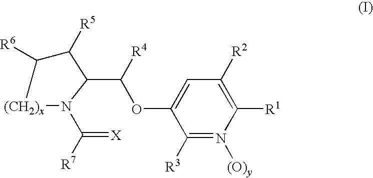
wherein
X is oxygen or sulfur;
R¹ and R² are each independently hydrogen, halogen, cyano, nitro, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₆-halocycloalkyl, C₃-C₆-cycloalkenyl, C₃-C₆-halocycloalkenyl, C₁-C₆-alkyl-C₃-C₆-cycloalkyl, ORⁱ, SRⁱ, S(═O)Rⁱ, S(═O)₂Rⁱ, NRⁱR^{j}, C(═O)ORⁱ, SiRⁱ_{z}R^{j}_{3-z} (z is 0 to 3), or
phenyl or a 5- to 6-membered heteroaromatic ring which may contain 1 to 4 heteroatoms selected from the group consisting of oxygen, nitrogen and sulfur, or a 3- to 7-membered saturated or partially unsaturated heterocyclic ring which may contain 1 to 3 heteroatoms selected from the group consisting of oxygen, nitrogen and sulfur,
wherein the carbon atoms in phenyl or in the heteroaromatic or heterocyclic ring may be substituted with 1 to 3 groups selected from the group consisting of halogen, amino, cyano, Rⁱ, ORⁱ, SRⁱ and nitro;
Rⁱ, R^{j} are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₈-halocycloalkyl, C₃-C₆-cycloalkenyl, C₃-C₆-halocycloalkenyl, C₁-C₆-haloalkoxy, C₁-C₄-alkoxy-C₁-C₆-alkyl, C₁-C₄-alkylthio-C₁-C₆-alkyl, C₁-C₄-alkylsulfinyl-C₁-C₆-alkyl, C₁-C₄-alkylsulfonyl-C₁-C₆-alkyl, C₁-C₄-haloalkoxy-C₁-C₆-alkyl, C₁-C₄-haloalkylthio-C₁-C₆-alkyl, (C₁-C₄-alkoxy)carbonyl-C₁-C₆-alkyl, (C₁-C₄-alkyl)amino-C₁-C₆-alkyl, di(C₁-C₄-alkyl)amino-C₁-C₆-alkyl, C₃-C₆-cycloalkyl-C₁-C₆-alkyl, phenyl-C₁-C₆-alkyl, or C₁-C₆-alkyl which is substituted with 1 to 3 cyano groups;
R³ is hydrogen, halogen or C₁-C₆-alkyl;
R⁴ is hydrogen or C₁-C₆-alkyl;
R⁵ and R⁶ are each independently hydrogen, halogen, cyano, or C₁-C₆-alkyl;
R⁷ is C₁-C₆-alkyl, C₂-C₆-alkenyl, C₂-C₆-alkynyl, C₃-C₆-cycloalkyl, C₃-C₆-cycloalkenyl, C₁-C₆-haloalkyl, C₂-C₆-haloalkenyl, C₂-C₆-haloalkynyl, C₃-C₆-halocycloalkyl, C₃-C₆-halocycloalkenyl, —C(=G)R^{a}, —C(=G)OR^{a}, —C(=G)NR^{a}R^{b}, —C(═NOR^{a})R^{b}, or C₃-C₆-cycloalkyl-C₁-C₆-alkyl; wherein
G is oxygen or sulfur;
R^{a}, R^{b} are each independently halogen, cyano, nitro, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₆-halocycloalkyl, C₃-C₆-cycloalkenyl, C₃-C₆-halocycloalkenyl, OR^{k}, SR^{k}, S(═O)R^{k}, S(═O)₂R^{k}, S(═O)₂NR^{k}R^{m}, C(═O)R^{k}, C(═O)OR^{k}, C(═O)NR^{k}R^{m}, C(═NOR^{k})R^{m}, —C(=G)NR^{k}—NR^{m}Rⁿ, SiR^{k}_{z}R^{m}_{3-z} wherein z is 0 to 3, or
R^{a}, R^{b} are each independently phenyl or a 5- to 6-membered heteroaromatic ring which may contain 1 to 4 heteroatoms selected from the group consisting of oxygen, nitrogen and sulfur, or a 4- to 7-membered saturated or partially unsaturated heterocyclic ring which may contain 1 to 3 heteroatoms selected from the group consisting of oxygen, nitrogen and sulfur, wherein the carbon atoms in phenyl or in the heteroaromatic or heterocyclic ring may be substituted with 1 to 5 halogens;
R^{k}, R^{m}, Rⁿ are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₈-halocycloalkyl, C₃-C₆-cycloalkenyl, or C₃-C₆-halocycloalkenyl; or
R⁷ is phenyl or a 3- to 7-membered saturated or partially unsaturated heterocyclic ring which may contain 1 to 3 heteroatoms selected from the group consisting of oxygen, sulfur and nitrogen or a 5- to 6-membered heteroaromatic ring system which may contain 1 to 4 heteroatoms selected from the group consisting of oxygen, nitrogen and sulfur, which phenyl, heterocyclic ring or heteroaromatic ring is bonded via a carbon atom of the ring or which phenyl, heterocyclic ring or heteroaromatic ring may be bonded via C₁-C₄-alkyl,
wherein phenyl or the heteroaromatic ring or the heterocyclic ring may be fused to a ring selected from the group consisting of phenyl and a 5- to 6-membered saturated, partially unsaturated or aromatic heterocyclic ring which may contain 1 to 3 heteroatoms selected from the group consisting of oxygen, nitrogen and sulfur,
wherein the above groups R⁷ are unsubstituted, or the hydrogen atoms in these groups may all or in part be replaced with any combination of groups selected from R^{c};
R^{c} is each independently halogen, cyano, nitro, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₈-halocycloalkyl, C₃-C₆-cycloalkenyl, C₃-C₈-halocycloalkenyl, OR^{k}, SR^{k}, S(═O)R^{k}, S(═O)₂R^{k}, NR^{k}R^{m}, N(OR^{k})R^{m}, —S(═O)₂NR^{k}R^{m}, C(═O)R^{k}, C(═O)OR^{k}, C(═O)NR^{k}R^{m}, C(═NOR^{k})R^{m}, —NR^{k}C(=G)R^{m}, —N(OR^{k})C(=G)R^{m}, —N[C(=G)R^{k}][C(=G)R^{m}], —NR^{k}C(=G)OR^{m}, —N(OR^{k})C(=G)OR^{m}, —C(=G)NR^{k}—NR^{m}Rⁿ, —NR^{k}SO₂R^{m}, SiR^{k}_{z}R^{m}_{3-z} wherein z is 0 to 3, or
R^{c} is each independently phenyl or a 5- to 6-membered heteroaromatic ring which may contain 1 to 4 heteroatoms selected from the group consisting of oxygen, nitrogen and sulfur, or a 4- to 7-membered saturated or partially unsaturated heterocyclic ring which may contain 1 to 3 heteroatoms selected from the group consisting of oxygen, nitrogen and sulfur, wherein the carbon atoms in phenyl or in the heteroaromatic or heterocyclic ring may be substituted with 1 to 5 halogens; wherein
R^{k}, R^{m}, Rⁿ are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₈-halocycloalkyl, C₃-C₆-cycloalkenyl, C₃-C₆-halocycloalkenyl;
x is 0 or 1; and
y is 0 or 1;
or a diastereomer, enantiomer or salt thereof, or
a 3-pyridyl derivative of formula II 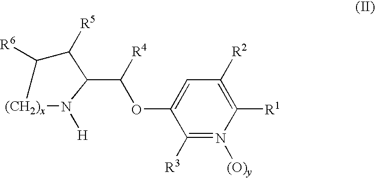
wherein
x, y are zero;
R⁴ is hydrogen or C₁-C₄-alkyl;
R¹, R² are each independently hydrogen, halogen, cyano, C₁-C₄-alkyl, C₁-C₄-haloalkyl, C₁-C₄-alkoxy, C₁-C₄-alkylthio, C₁-C₄-alkylsulfinyl, C₁-C₄-alkylsulfonyl, C₁-C₄-haloalkylthio, C₁-C₄-haloalkylsulfinyl, C₁-C₄-haloalkylsulfonyl; and
R³, R⁵, R⁶ are hydrogen
or a diastereomer, enantiomer or salt thereof
and a pesticide selected from the group consisting of organo(thio)phosphates, carbamates, pyrethroids, growth regulators, neonicotinoids, nicotinic receptor agonists/antagonists compounds, GABA antagonist compounds, macrocyclic lactone insecticides, METI I, II and III compounds, oxidative phosphorylation inhibitor compounds, moulting disruptor compounds, mixed function oxidase inhibitor compounds, sodium channel blocker compounds, benclothiaz, bifenazate, cartap, flonicamid, pyridalyl, pymetrozine, sulfur, thiocyclam, flubendiamide, cyenopyrafen, flupyrazofos, cyflumetofen, amidoflumet, N—R′-2,2-dihalo-1-R″ cyclo-propanecarboxamide-2-(2,6-dichloro-α,α,α-tri-fluoro-p-tolyl)hydrazone and N—R′-2,2-di(R′″)propionamide-2-(2,6-dichloro-α,α,α-trifluoro-p-tolyl)-hydrazone, wherein R′ is methyl or ethyl, halo is chloro or bromo, R″ is hydrogen or methyl and R′″ is methyl or ethyl, and anthranilamide compounds of formula Γ³ 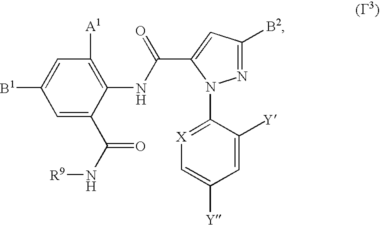
wherein A¹ is CH₃, Cl, Br, I, X is C—H, C—Cl, C—F or N, Y′ is F, Cl, or Br, Y″ is F, Cl, CF₃, B¹ is hydrogen, Cl, Br, I, CN, B² is Cl, Br, CF₃, OCH₂CF₃, OCF₂H, and R^{B} is hydrogen, CH₃ or CH(CH₃)₂.
